(19) United States
(12) Patent Application Publication
(10) Pub. No.: US 2023/0302014 A1
(43) Pub. Date: Sep. 28, 2023

(54) USES OF GLUCOCORTICOID RECEPTOR (GR) ANTAGONIST AND ANDROGEN RECEPTOR (AR) DEGRADER COMBINATIONS

(71) Applicant: ORIC Pharmaceuticals, Inc., South San Francisco, CA (US)

(72) Inventors: Haiying ZHOU, San Bruno, CA (US); Lori S. FRIEDMAN, San Carlos, CA (US); Shravani BARKUND, San Francisco, CA (US); Anneleen DAEMEN, Belmont, CA (US)

(21) Appl. No.: 18/007,369

(22) PCT Filed: Aug. 3, 2021
(86) PCT No.: PCT/US2021/044255

§ 371 Date: Jan. 30, 2023

Related U.S. Application Data
(60) Provisional application No. 63/226,102, filed on Jul. 27, 2021.
(60) Provisional application No. 63/061,013, filed on Aug. 4, 2020.

(51) Int. Cl. A61K 31/567; A61K 31/403; A61K 31/513; A61K 31/437; A61P 35/04

(52) U.S. Cl. CPC A61K 31/567; A61K 31/403; A61K 31/437; A61K 31/513; A61P 35/04

(57) ABSTRACT

Disclosed herein are methods of treating prostate cancer in a subject in need thereof, the method comprising administering to the subject a therapeutically effective amount of (i) a glucocorticoid receptor (GR) antagonist and (ii) an androgen receptor (AR) degrader.

### CROSS-REFERENCE

This application claims the benefit of U.S. Provisional Application Serial No. 63/061,013 filed Aug. 4, 2020 and U.S. Provisional Application Serial No. 63/226,102 filed Jul. 27, 2021; which are hereby incorporated by reference in their entirety.

### BACKGROUND OF THE INVENTION

A need exists in the art for an effective treatment of cancer and neoplastic disease.

### SUMMARY OF THE INVENTION

Disclosed herein is a method of treating prostate cancer in a subject in need thereof, the method comprising administering to the subject a therapeutically effective amount of (i) a glucocorticoid receptor (GR) antagonist and (ii) an androgen receptor (AR) degrader. In some embodiments of a method of treating prostate cancer, one or more cells comprising the prostate cancer in the subject exhibits an elevated expression of glucocorticoid receptor (GR) prior to the administration of the therapeutically effective amount of the glucocorticoid receptor (GR) antagonist and the androgen receptor (AR) degrader. In some embodiments of a method of treating prostate cancer, the glucocorticoid receptor (GR) antagonist and the androgen receptor (AR) degrader are administered to the subject in need thereof concurrently. In some embodiments of a method of treating prostate cancer, the glucocorticoid receptor (GR) antagonist and the androgen receptor (AR) degrader are administered to the subject in need thereof consecutively. In some embodiments of a method of treating prostate cancer, the prostate cancer in the subject is metastatic prostate cancer. In some embodiments of a method of treating prostate cancer, the prostate cancer in the subject is metastatic castration-resistant prostate cancer. In some embodiments of a method of treating prostate cancer, the prostate cancer in the subject is localized high risk prostate cancer, recurrent prostate cancer, non-metastatic CRPC (nmCRPC), non-metastatic castration-sensitive prostate cancer, or metastatic castration-sensitive prostate cancer. In some embodiments of a method of treating prostate cancer, the glucocorticoid receptor (GR) antagonist is Compound 1:

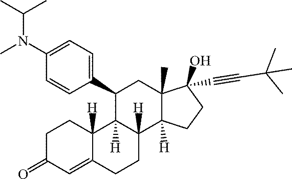

or a pharmaceutically acceptable salt thereof. In some embodiments of a method of treating prostate cancer, the glucocorticoid receptor (GR) antagonist is mifepristone, cyproterone acetate, relacorilant (CORT125134), exicorilant (CORT125281), miricorilant (CORT118335), CORT113176, CORT108297, PT150 (formerly Org34517), PT157, or PT162. In some embodiments of a method of treating prostate cancer, the androgen receptor (AR) degrader is ARV-110

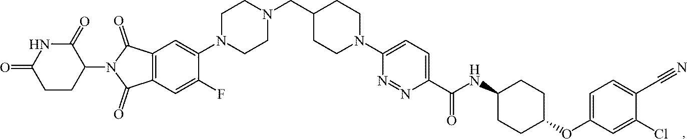

or a pharmaceutically acceptable salt, solvate, or stereoisomer thereof.

Also disclosed herein is a glucocorticoid receptor (GR) antagonist for use in combination with an androgen receptor (AR) degrader in a method of treating prostate cancer in a subject. In some embodiments of a glucocorticoid receptor (GR) antagonist for use in a method of treating prostate cancer, the prostate cancer in the subject is metastatic prostate cancer. In some embodiments of a glucocorticoid receptor (GR) antagonist for use in a method of treating prostate cancer, the prostate cancer in the subject is metastatic castration-resistant prostate cancer. In some embodiments of a glucocorticoid receptor (GR) antagonist for use in a method of treating prostate cancer, the prostate cancer in the subject is localized high risk prostate cancer, recurrent prostate cancer, non-metastatic CRPC (nmCRPC), non-metastatic castration-sensitive prostate cancer, or metastatic castration-sensitive prostate cancer. In some embodiments of a glucocorticoid receptor (GR) antagonist for use in a method of treating prostate cancer, the glucocorticoid receptor (GR) antagonist is Compound 1:

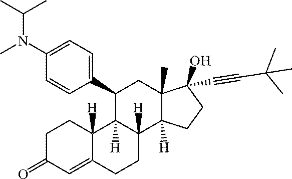

or a pharmaceutically acceptable salt thereof. In some embodiments of a glucocorticoid receptor (GR) antagonist for use in a method of treating prostate cancer, the glucocorticoid receptor (GR) antagonist is mifepristone, cyproterone acetate, relacorilant (CORT125134), exicorilant (CORT125281), miricorilant (CORT118335), CORT113176, CORT108297, PT150 (formerly Org34517), PT157, or PT162. In some embodiments of a glucocorticoid receptor (GR) antagonist for use in a method of treating prostate cancer, the androgen receptor (AR) degrader is ARV-110

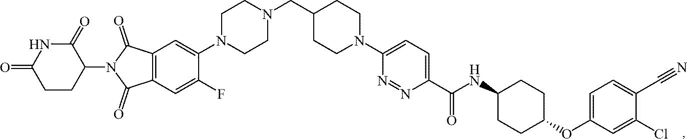

or a pharmaceutically acceptable salt, solvate, or stereoisomer thereof.

Also disclosed herein is a method of treating prostate cancer in a subject in need thereof, the method comprising administering to the subject a therapeutically effective amount of (i) a glucocorticoid receptor (GR) antagonist and (ii) an androgen receptor (AR) degrader. In some embodiments of a method of treating prostate cancer, one or more cells comprising the prostate cancer in the subject exhibits an elevated expression of glucocorticoid receptor (GR) prior to the administration of the therapeutically effective amount of the GR antagonist and the AR degrader. In some embodiments of a method of treating prostate cancer, the subject in need thereof has not been previously administered an AR degrader prior to the administration of the therapeutically effective amounts of the GR antagonist and the AR degrader. In some embodiments of a method of treating prostate cancer, the subject in need thereof has not been previously administered an AR degrader for at least a 6-month period prior to the administration of the therapeutically effective amounts of the GR antagonist and the AR degrader. In some embodiments of a method of treating prostate cancer, the subject in need thereof has not been previously administered an AR degrader for at least a one-year period prior to the administration of the therapeutically effective amounts of the GR antagonist and the AR degrader. In some embodiments of a method of treating prostate cancer, the subject in need thereof has not been previously administered an AR degrader for at least a two-year period prior to the administration of the therapeutically effective amounts of the GR antagonist and the AR degrader. In some embodiments of a method of treating prostate cancer, the subject in need thereof has been previously administered a AR degrader prior to the administration of the therapeutically effective amounts of the GR antagonist and the AR degrader. In some embodiments of a method of treating prostate cancer, one or more cells comprising the prostate cancer in the subject in need thereof has become resistant to the AR degrader prior to the administration of the therapeutically effective amounts of the GR antagonist and the AR degrader. In some embodiments of a method of treating prostate cancer, the AR degrader has been administered to the subject for a period of time of between about 1 month and about 2 years prior to the administration of the therapeutically effective amounts of the GR antagonist and the AR degrader. In some embodiments of a method of treating prostate cancer, the AR degraders are the same. In some embodiments of a method of treating prostate cancer, the AR degraders are different. In some embodiments of a method of treating prostate cancer, the GR antagonist and the AR degrader are administered to the subject in need thereof concurrently. In some embodiments of a method of treating prostate cancer, the GR antagonist and the AR degrader are administered to the subject in need thereof consecutively. In some embodiments of a method of treating prostate cancer, the prostate cancer in the subject is metastatic prostate cancer. In some embodiments of a method of treating prostate cancer, the prostate cancer in the subject is metastatic castration-resistant prostate cancer. In some embodiments of a method of treating prostate cancer, the prostate cancer in the subject is Localized high risk prostate cancer, recurrent prostate cancer, non-metastatic CRPC (nmCRPC), non-metastatic castration-sensitive prostate cancer, or metastatic castration-sensitive prostate cancer. In some embodiments of a method of treating prostate cancer, the prostate cancer in the subject was previously treated with one or more additional therapeutic agents. In some embodiments of a method of treating prostate cancer, the one or more additional therapeutic agents is selected from one or more androgen receptor inhibitors, one or more chemotherapeutic agents, and one or more immunotherapy agents, or any combinations thereof. In some embodiments of a method of treating prostate cancer, the one or more androgen receptor inhibitors is selected from 3,3′-diindolylmethane (DIM), abiraterone acetate, bexlosteride, bicalutamide, dutasteride, epristeride, enzalutamide, apalutamide, finasteride, flutamide, izonsteride, ketoconazole, N-butylbenzene-sulfonamide, nilutamide, megestrol, steroidal antiandrogens, and turosteride, or any combinations thereof.

Also disclosed herein is a method of treating prostate cancer in a subject in need thereof, wherein the subject has been previously administered chemotherapy comprising a first androgen receptor (AR) degrader, the method comprising administering to the subject a therapeutically effective amount of (i) a glucocorticoid receptor (GR) antagonist and (ii) a second androgen receptor (AR) degrader. In some embodiments of a method of treating prostate cancer, one or more cells comprising the prostate cancer in the subject exhibits an elevated expression of glucocorticoid receptor (GR) prior to the administration of the therapeutically effective amount of the GR antagonist and the second AR degrader. In some embodiments of a method of treating prostate cancer, one or more cells comprising the prostate cancer in the subject has become resistant to the first AR degrader. In some embodiments of a method of treating prostate cancer, the subject has been previously administered chemotherapy comprising the first AR degrader for a period of time of between about 1 month and about 2 years prior to the administration of the therapeutically effective amounts of the GR antagonist and the second AR degrader. In some embodiments of a method of treating prostate cancer, the subject has been previously administered chemotherapy comprising the first AR degrader within one month prior to the administration of the therapeutically effective amounts of the GR antagonist and the second AR degrader. In some embodiments of a method of treating prostate cancer, the subject has been previously administered chemotherapy comprising the first AR degrader within one year prior to the administration of the therapeutically effective amounts of the GR antagonist and the second AR degrader. In some embodiments of a method of treating prostate cancer, the first and the second AR degraders are the same. In some embodiments of a method of treating prostate cancer, the first and the second AR degraders are different. In some embodiments of a method of treating prostate cancer, the GR antagonist and the second AR degrader are administered to the subject concurrently. In some embodiments of a method of treating prostate cancer, the GR antagonist and the second AR degrader are administered to the subject consecutively. In some embodiments of a method of treating prostate cancer, the prostate cancer in the subject is metastatic prostate cancer. In some embodiments of a method of treating prostate cancer, the prostate cancer in the subject is metastatic castration-resistant prostate cancer. In some embodiments of a method of treating prostate cancer, the prostate cancer in the subject is Localized high risk prostate cancer, recurrent prostate cancer, non-metastatic CRPC (nmCRPC), non-metastatic castration-sensitive prostate cancer, or metastatic castration-sensitive prostate cancer. In some embodiments of a method of treating prostate cancer, the prostate cancer in the subject was previously treated with one or more additional therapeutic agents. In some embodiments of a method of treating prostate cancer, the one or more additional therapeutic agents is selected from one or more androgen receptor inhibitors, one or more chemotherapeutic agents, and one or more immunotherapy agents, or any combinations thereof. In some embodiments of a method of treating prostate cancer, the one or more androgen receptor inhibitors is selected from 3,3′-diindolylmethane (DIM), abiraterone acetate, bexlosteride, bicalutamide, dutasteride, epristeride, enzalutamide, apalutamide, finasteride, flutamide, izonsteride, ketoconazole, N-butylbenzene-sulfonamide, nilutamide, megestrol, steroidal antiandrogens, and turosteride, or any combinations thereof.

Also disclosed herein is a method of treating prostate cancer in a subject in need thereof, the method comprising administering to the subject a therapeutically effective amount of (i) a glucocorticoid receptor (GR) antagonist and (ii) an androgen receptor (AR) degrader, wherein the subject has not been previously administered chemotherapy comprising an androgen receptor (AR) degrader prior to the administration of the therapeutically effective amounts of the GR antagonist and the AR degrader. In some embodiments of a method of treating prostate cancer, one or more cells comprising the prostate cancer in the subject exhibits an elevated expression of glucocorticoid receptor (GR) prior to the administration of the therapeutically effective amount of the GR antagonist and the AR degrader. In some embodiments of a method of treating prostate cancer, the subject in need thereof has not been previously administered an AR degrader for at least a 6-month period prior to the administration of the therapeutically effective amounts of the GR antagonist and the AR degrader. In some embodiments of a method of treating prostate cancer, the subject in need thereof has not been previously administered an AR degrader for at least a one-year period prior to the administration of the therapeutically effective amounts of the GR antagonist and the AR degrader. In some embodiments of a method of treating prostate cancer, the subject in need thereof has not been previously administered an AR degrader for at least a two-year period prior to the administration of the therapeutically effective amounts of the GR antagonist and the AR degrader. In some embodiments of a method of treating prostate cancer, the GR antagonist and the AR degrader are administered to the subject concurrently. In some embodiments of a method of treating prostate cancer, the GR antagonist and the AR degrader are administered to the subject consecutively. In some embodiments of a method of treating prostate cancer, the prostate cancer in the subject is metastatic prostate cancer. In some embodiments of a method of treating prostate cancer, the prostate cancer in the subject is metastatic castration-resistant prostate cancer. In some embodiments of a method of treating prostate cancer, the prostate cancer in the subject is Localized high risk prostate cancer, recurrent prostate cancer, non-metastatic CRPC (nmCRPC), non-metastatic castration-sensitive prostate cancer, or metastatic castration-sensitive prostate cancer. In some embodiments of a method of treating prostate cancer, the prostate cancer in the subject was previously treated with one or more additional therapeutic agents. In some embodiments of a method of treating prostate cancer, the one or more additional therapeutic agents is selected from one or more androgen receptor inhibitors, one or more chemotherapeutic agents, and one or more immunotherapy agents, or any combinations thereof. In some embodiments of a method of treating prostate cancer, the one or more androgen receptor inhibitors is selected from 3,3′-diindolylmethane (DIM), abiraterone acetate, bexlosteride, bicalutamide, dutasteride, epristeride, enzalutamide, apalutamide, finasteride, flutamide, izonsteride, ketoconazole, N-butylbenzene-sulfonamide, nilutamide, megestrol, steroidal antiandrogens, and turosteride, or any combinations thereof.

Also disclosed herein is a method of treating prostate cancer in a subject in need thereof, the method comprising (a) measuring the expression of glucocorticoid receptor (GR) in one or more of the cells comprising the prostate cancer, and (b) if one or more of the cells comprising the prostate cancer exhibits increased expression level of GR, administering to the subject a therapeutically effective amounts of (i) a glucocorticoid receptor (GR) antagonist and (ii) an androgen receptor (AR) degrader. In some embodiments of a method of treating prostate cancer, the subject in need thereof has not been previously administered an AR degrader prior to the administration of the therapeutically effective amounts of the GR antagonist and the AR degrader. In some embodiments of a method of treating prostate cancer, the subject in need thereof has not been previously administered an AR degrader for at least a 6-month period prior to the administration of the therapeutically effective amounts of the GR antagonist and the AR degrader. In some embodiments of a method of treating prostate cancer, the subject in need thereof has not been previously administered an AR degrader for at least a one-year period prior to the administration of the therapeutically effective amounts of the GR antagonist and the AR degrader. In some embodiments of a method of treating prostate cancer, the subject in need thereof has not been previously administered an AR degrader for at least a two-year period prior to the administration of the therapeutically effective amounts of the GR antagonist and the AR degrader. In some embodiments of a method of treating prostate cancer, the subject in need thereof has been previously administered an AR degrader prior to the administration of the therapeutically effective amounts of the GR antagonist and the AR degrader. In some embodiments of a method of treating prostate cancer, one or more cells comprising the prostate cancer in the subject in need thereof has become resistant to the AR degrader prior to the administration of the therapeutically effective amounts of the GR antagonist and the AR degrader. In some embodiments of a method of treating prostate cancer, the AR degrader has been administered to the subject for a period of time of between about 1 month and about 2 years prior to the administration of the therapeutically effective amounts of the GR antagonist and the AR degrader. In some embodiments of a method of treating prostate cancer, the AR degraders are the same. In some embodiments of a method of treating prostate cancer, the AR degraders are different. In some embodiments of a method of treating prostate cancer, the GR antagonist and the AR degrader are administered to the subject in need thereof concurrently. In some embodiments of a method of treating prostate cancer, the GR antagonist and the AR degrader are administered to the subject in need thereof consecutively. In some embodiments of a method of treating prostate cancer, the prostate cancer in the subject is metastatic prostate cancer. In some embodiments of a method of treating prostate cancer, the prostate cancer in the subject is metastatic castration-resistant prostate cancer. In some embodiments of a method of treating prostate cancer, the prostate cancer in the subject is Localized high risk prostate cancer, recurrent prostate cancer, non-metastatic CRPC (nmCRPC), non-metastatic castration-sensitive prostate cancer, or metastatic castration-sensitive prostate cancer. In some embodiments of a method of treating prostate cancer, the prostate cancer in the subject was previously treated with one or more additional therapeutic agents. In some embodiments of a method of treating prostate cancer, the one or more additional therapeutic agents is selected from one or more androgen receptor inhibitors, one or more chemotherapeutic agents, and one or more immunotherapy agents, or any combinations thereof. In some embodiments of a method of treating prostate cancer, the one or more androgen receptor inhibitors is selected from 3,3′-diindolylmethane (DIM), abiraterone acetate, bexlosteride, bicalutamide, dutasteride, epristeride, enzalutamide, apalutamide, finasteride, flutamide, izonsteride, ketoconazole, N-butylbenzene-sulfonamide, nilutamide, megestrol, steroidal antiandrogens, and turosteride, or any combinations thereof.

Also disclosed herein is a method of treating prostate cancer in a subject in need thereof, the method comprising (a) administering to the subject an androgen antagonist (AR) degrader for a period of time, (b) measuring the expression of glucocorticoid receptor (GR) in one or more of the cells comprising the prostate cancer following administration of the AR degrader for the period of time, and (c) if one or more of the cells comprising the prostate cancer exhibits an increased expression level of GR following administration of the AR degrader for a period of time, administering to the subject a therapeutically effective amount of a glucocorticoid receptor (GR) antagonist. In some embodiments of a method of treating prostate cancer, the period of time is between about 1 month and about 2 years. In some embodiments of a method of treating prostate cancer, the method further comprises administering to the subject a therapeutically effective amount of an AR degrader in combination with the therapeutically effective amount of a GR antagonist. In some embodiments of a method of treating prostate cancer, the AR degraders are the same. In some embodiments of a method of treating prostate cancer, the AR degraders are different. In some embodiments of a method of treating prostate cancer, the GR antagonist and the AR degrader are administered to the subject in need thereof concurrently. In some embodiments of a method of treating prostate cancer, the GR antagonist and the AR degrader are administered to the subject in need thereof consecutively. In some embodiments of a method of treating prostate cancer, the prostate cancer in the subject is metastatic prostate cancer. In some embodiments of a method of treating prostate cancer, the prostate cancer in the subject is metastatic castration-resistant prostate cancer. In some embodiments of a method of treating prostate cancer, the prostate cancer in the subject is Localized high risk prostate cancer, recurrent prostate cancer, non-metastatic CRPC (nmCRPC), non-metastatic castration-sensitive prostate cancer, or metastatic castration-sensitive prostate cancer. In some embodiments of a method of treating prostate cancer, the prostate cancer in the subject was previously treated with one or more additional therapeutic agents. In some embodiments of a method of treating prostate cancer, the one or more additional therapeutic agents is selected from one or more androgen receptor inhibitors, one or more chemotherapeutic agents, and one or more immunotherapy agents, or any combinations thereof. In some embodiments of a method of treating prostate cancer, the one or more androgen receptor inhibitors is selected from 3,3′-diindolylmethane (DIM), abiraterone acetate, bexlosteride, bicalutamide, dutasteride, epristeride, enzalutamide, apalutamide, finasteride, flutamide, izonsteride, ketoconazole, N-butylbenzene-sulfonamide, nilutamide, megestrol, steroidal antiandrogens, and turosteride, or any combinations thereof.

Also disclosed herein is a method of treating prostate cancer in a subject in need thereof, the method comprising administering to the subject a therapeutically effective amount of (i) a second glucocorticoid receptor (GR) antagonist and (ii) an androgen receptor (AR) degrader, wherein the subject has been previously administered chemotherapy comprising (a) a first glucocorticoid receptor (GR) antagonist and (b) an androgen receptor (AR) inhibitor prior to the administration of the therapeutically effective amounts of the second GR antagonist and the AR degrader. In some embodiments of a method of treating prostate cancer, the AR inhibitor is 3,3′-diindolylmethane (DIM), abiraterone acetate, bexlosteride, bicalutamide, dutasteride, epristeride, enzalutamide, apalutamide, finasteride, flutamide, izonsteride, ketoconazole, N-butylbenzene-sulfonamide, nilutamide, megestrol, steroidal antiandrogens, and turosteride, or any combinations thereof. In some embodiments of a method of treating prostate cancer, the AR inhibitor is enzalutamide. In some embodiments of a method of treating prostate cancer, the chemotherapy comprising the first GR antagonist and the AR inhibitor has been administered to the subject for a period of time of between about 1 month and about 2 years prior to the administration of the therapeutically effective amounts of the second GR antagonist and the AR degrader. In some embodiments of a method of treating prostate cancer, one or more cells comprising the prostate cancer in the subject in need thereof has become resistant to the AR inhibitor prior to the administration of the therapeutically effective amounts of the second GR antagonist and the AR degrader. In some embodiments of a method of treating prostate cancer, the second GR antagonist and AR degrader are administered once the one or more cells comprising the prostate cancer in the subject in need thereof has become resistant to the AR inhibitor. In some embodiments of a method of treating prostate cancer, the first GR antagonist and the second GR antagonists are the same. In some embodiments of a method of treating prostate cancer, the first GR antagonist and the second GR antagonists are different. In some embodiments of a method of treating prostate cancer, the second GR antagonist and the AR degrader are administered to the subject in need thereof concurrently. In some embodiments of a method of treating prostate cancer, the second GR antagonist and the AR degrader are administered to the subject in need thereof consecutively. In some embodiments of a method of treating prostate cancer, the prostate cancer in the subject is metastatic prostate cancer. In some embodiments of a method of treating prostate cancer, the prostate cancer in the subject is metastatic castration-resistant prostate cancer. In some embodiments of a method of treating prostate cancer, the prostate cancer in the subject is Localized high risk prostate cancer, recurrent prostate cancer, non-metastatic CRPC (nmCRPC), non-metastatic castration-sensitive prostate cancer, or metastatic castration-sensitive prostate cancer. In some embodiments of a method of treating prostate cancer, the prostate cancer in the subject was previously treated with one or more additional therapeutic agents. In some embodiments of a method of treating prostate cancer, the one or more additional therapeutic agents is selected from one or more androgen receptor inhibitors, one or more chemotherapeutic agents, and one or more immunotherapy agents, or any combinations thereof. In some embodiments of a method of treating prostate cancer, the one or more androgen receptor inhibitors is selected from 3,3′-diindolylmethane (DIM), abiraterone acetate, bexlosteride, bicalutamide, dutasteride, epristeride, enzalutamide, apalutamide, finasteride, flutamide, izonsteride, ketoconazole, N-butylbenzene-sulfonamide, nilutamide, megestrol, steroidal antiandrogens, and turosteride, or any combinations thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

FIG. 1A depicts the mRNA levels of AR in prostate cancer cell lines.

FIG. 1B depicts the mRNA levels of GR in prostate cancer cell lines.

FIG. 1C depicts the mRNA levels of AR-V1 in prostate cancer cell lines.

FIG. 1D depicts the mRNA levels of AR-V7 in prostate cancer cell lines.

FIG. 2A depicts the structures of AR degraders ARD1 and ARD2.

FIG. 2B depicts the western blot showing AR and GR protein levels 3 days post increasing dosing of ARD1 treatment in LNCaP and CWR22PC cells. UT, untreated.

FIG. 2C depicts the RT-qPCR showing AR mRNA levels 3 days post increasing dosing of ARD1 treatment in LNCaP and CWR22PC cells.

FIG. 2D depicts the western blot showing AR and GR protein levels 3 days post increasing dosing of ARD2 treatment in LNCaP and CWR22PC cells. UT, untreated.

FIG. 2E depicts the RT-qPCR showing ARmRNA levels 3 days post increasing dosing of ARD2 treatment in LNCaP and CWR22PC cells.

FIG. 3A depicts how Enzalutamide (Enz), ARD1, and ARD2 inhibit AR target gene KLK3 induced by 100 pM R1881 in LNCaP cells 24 hours post treatment.

FIG. 3B depicts how Enzalutamide (Enz), ARD1, and ARD2 inhibit AR target gene KLK4 induced by 100 pM R1881 in LNCaP cells 24 hours post treatment.

FIG. 3C depicts how Enzalutamide (Enz), ARD1, and ARD2 inhibit AR target gene NKX3.1 induced by 100 pM R1881 in LNCaP cells 24 hours post treatment.

FIG. 3D depicts how Enzalutamide (Enz), ARD1, and ARD2 inhibit AR target gene FKBP5 induced by 100 pM R1881 in LNCaP cells 24 hours post treatment.

FIG. 3E depicts how Enzalutamide (Enz), ARD1, and ARD2 inhibit AR target gene KLK3 induced by 100 pM R1881 in CWR22PC cells 24 hours post treatment.

FIG. 3F depicts how Enzalutamide (Enz), ARD1, and ARD2 inhibit AR target gene KLK4 induced by 100 pM R1881 in CWR22PC cells 24 hours post treatment.

FIG. 3G depicts how Enzalutamide (Enz), ARD1, and ARD2 inhibit AR target gene NKX3.1 induced by 100 pM R1881 in CWR22PC cells 24 hours post treatment.

FIG. 3H depicts how Enzalutamide (Enz), ARD1, and ARD2 inhibit AR target gene FKBP5 induced by 100 pM R1881 in CWR22PC cells 24 hours post treatment.

FIG. 4 depicts the PSA levels in CWR22PC cells at 22 days post indicated treatment conditions (vehicle, R1881, R1881+ARD1, and R1881+ARD2).

FIG. 5A depicts the GR mRNA levels in LNCaP and CWR22PC cells post ARD1 treatment at 0, 6, 10, 13, and 15 days (d).

FIG. 5B depicts the GRmRNA levels in LNCaP and CWR22PC cells post ARD2 treatment at 0, 6, 10, 13, and 15 days (d).

FIG. 5C depicts the AR and GR protein levels in LNCaP cells post ARD1 or ARD2 treatment at indicated days.

FIG. 6A depicts the mRNA levels of GR target gene GILZ in LNCaP and CWR22PC cells post ARD1 treatment at 0, 6, 10, 13, and 15 days (d).

FIG. 6B depicts the mRNA levels of GR target gene PER1 in LNCaP and CWR22PC cells post ARD1 treatment at 0, 6, 10, 13, and 15 days (d).

FIG. 6C depicts the mRNA levels of GR target gene KLF9 in LNCaP and CWR22PC cells post ARD1 treatment at 0, 6, 10, 13, and 15 days (d).

FIG. 6D depicts the mRNA levels of GR target gene SGK1 in LNCaP and CWR22PC cells post ARD1 treatment at 0, 6, 10, 13, and 15 days (d).

FIG. 6E depicts the mRNA levels of GR target gene GILZ in LNCaP and CWR22PC cells post ARD2 treatment at 0, 6, 10, 13, and 15 days (d).

FIG. 6F depicts the mRNA levels of GR target gene PER1 in LNCaP and CWR22PC cells post ARD2 treatment at 0, 6, 10, 13, and 15 days (d).

FIG. 6G depicts the mRNA levels of GR target gene KLF9 in LNCaP and CWR22PC cells post ARD2 treatment at 0, 6, 10, 13, and 15 days (d).

FIG. 6H depicts the mRNA levels of GR target gene SGK1 in LNCaP and CWR22PC cells post ARD2 treatment at 0, 6, 10, 13, and 15 days (d).

FIG. 7A depicts the mRNA of AR target gene KLK4 in CWR22PC and 22Rv1 cells 48 hours post R1881 (100 pM), DEX (30 nM dexamethasone), ARD1 (1 µM), ARD2 (1 µM), and compound 1 (0.5 µM) treatment as indicated.

FIG. 7B depicts the mRNA of GR target gene KLF9 in CWR22PC and 22Rv1 cells 48 hours post R1881 (100 pM), DEX (30 nM dexamethasone), ARD1 (1 µM), ARD2 (1 µM), and compound 1 (0.5 µM) treatment as indicated.

FIG. 7C depicts the mRNA of GR and AR target gene FKBP5 in CWR22PC and 22Rv1 cells 48 hours post R1881 (100 pM), DEX (30 nM dexamethasone), ARD1 (1 µM), ARD2 (1 µM), and compound 1 (0.5 µM) treatment as indicated.

FIG. 7D depicts the mRNA of GR and AR target gene PER1 in CWR22PC and 22Rv1 cells 48 hours post R1881 (100 pM), DEX (30 nM dexamethasone), ARD1 (1 µM), ARD2 (1 µM), and compound 1 (0.5 µM) treatment as indicated.

FIG. 7E depicts the mRNA of GR and AR target gene SGK1 in CWR22PC and 22Rv1 cells 48 hours post R1881 (100 pM), DEX (30 nM dexamethasone), ARD1 (1 µM), ARD2 (1 µM), and compound 1 (0.5 µM) treatment as indicated.

FIG. 8A depicts the number of CWR22PC cells under the indicated treatment conditions for 19 days (vehicle, R1881, R1881+ARD1, R1881+ARD1+DEX, and R1881+ARD1+DEX+compound 1).

FIG. 8B depicts the PSA levels in CWR22PC cells under the indicated treatment conditions for 19 days (vehicle, R1881, R1881+ARD1, R1881+ARD1+ DEX, and R1881+ARD1+ DEX +compound 1). Assays were done in CSS media, R1881: 100 pM synthetic AR ligand; DEX: 30 nM dexamethasone; compound 1: 0.5 µM; ARD1: 1 µM.

FIG. 9A depicts the mRNA levels of AR target gene NKX3.1 in CWR22PC cells after 19 days of the indicated treatment conditions (vehicle, R1881, R1881+ARD1, R1881+ARD1+ DEX, and R1881+ARD1+ DEX +compound 1).

FIG. 9B depicts the mRNA levels of AR target gene KLK4 in CWR22PC cells after 19 days of the indicated treatment conditions (vehicle, R1881, R1881+ARD1, R1881+ARD1+ DEX, and R1881+ARD1+ DEX +compound 1).

FIG. 9C depicts the mRNA levels of AR and GR target gene FKBP5 in CWR22PC cells after 19 days of the indicated treatment conditions (vehicle, R1881, R1881+ARDI1, R1881+ARD1+ DEX, and R1881+ARD1+ DEX +compound 1).

FIG. 9D depicts the mRNA levels of GR target gene KLF9 in CWR22PC cells after 19 days of the indicated treatment conditions (vehicle, R1881, R1881+ARD1, R1881+ARD1+ DEX, and R1881+ARD1+ DEX +compound 1).

FIG. 10A depicts the number of CWR22PC cells under the indicated treatment conditions for 19 days (vehicle, R1881, R1881+ARD2, R1881+ARD2+ DEX, and R1881+ARD2+ DEX +compound 1)

FIG. 10B depicts the PSA levels in CWR22PC cells under the indicated treatment conditions for 19 days (vehicle, R1881, R1881+ARD2, R1881+ARD2+ DEX, and R1881+ARD2+ DEX +compound 1). Assays were done in CSS media, R1881: 100 pM synthetic AR ligand; DEX: 30 nM dexamethasone; compound 1: 0.5 µM; ARD2: 1 µM.

FIG. 11A depicts the mRNA levels of AR target gene NKX3.1 in CWR22PC cells after 19 days of the indicated treatment conditions (vehicle, R1881, R1881+ARD2, R1881+ARD2+ DEX, and R1881+ARD2+ DEX +compound 1).

FIG. 11B depicts the mRNA levels of AR target gene KLK4 in CWR22PC cells after 19 days of the indicated treatment conditions (vehicle, R1881, R1881+ARD2, R1881+ARD2+ DEX, and R1881+ARD2+ DEX +compound 1).

FIG. 11C depicts the mRNA levels of AR and GR target gene FKBP5 in CWR22PC cells after 19 days of the indicated treatment conditions (vehicle, R1881, R1881+ARD2, R1881+ARD2+ DEX, and R1881+ARD2+ DEX +compound 1).

FIG. 11D depicts the mRNA levels of GR target gene KLF9 in CWR22PC cells after 19 days of the indicated treatment conditions (vehicle, R1881, R1881+ARD2, R1881+ARD2+ DEX, and R1881+ARD2+ DEX +compound 1).

FIG. 12A depicts the structure of AR degrader ARV-110.

FIG. 12B depicts the western blot showing AR and GR protein levels 3 days post increasing dosing of ARV-110 treatment in LNCaP and CWR22PC cells. UT, untreated.

FIG. 12C depicts the RT-qPCR showing AR mRNA levels 3 days post increasing dosing of ARV-110 treatment in LNCaP and CWR22PC cells.

FIG. 13A depicts how Enzalutamide (Enz) and ARV-110 inhibit AR target gene KLK3 induced by 100 pM R1881 in LNCaP cells 24 hours post treatment.

FIG. 13B depicts how Enzalutamide (Enz) and ARV-110 inhibit AR target gene KLK4 induced by 100 pM R1881 in LNCaP cells 24 hours post treatment.

FIG. 13C depicts how Enzalutamide (Enz) and ARV-110 inhibit AR target gene NKX3.1 induced by 100 pM R1881 in LNCaP cells 24 hours post treatment.

FIG. 13D depicts how Enzalutamide (Enz) and ARV-110 inhibit AR target gene FKBP5 induced by 100 pM R1881 in LNCaP cells 24 hours post treatment.

FIG. 13E depicts how Enzalutamide (Enz) and ARV-110 inhibit AR target gene KLK3 induced by 100 pM R1881 in CWR22PC cells 24 hours post treatment.

FIG. 13F depicts how Enzalutamide (Enz) and ARV-110 inhibit AR target gene KLK4 induced by 100 pM R1881 in CWR22PC cells 24 hours post treatment.

FIG. 13G depicts how Enzalutamide (Enz) and ARV-110 inhibit AR target gene NKX3.1 induced by 100 pM R1881 in CWR22PC cells 24 hours post treatment.

FIG. 13H depicts how Enzalutamide (Enz) and ARV-110 inhibit AR target gene FKBP5 induced by 100 pM R1881 in CWR22PC cells 24 hours post treatment.

FIG. 14 depicts the GR mRNA levels in LNCaP and CWR22PC cells post ARV-110 treatment at 0, 6, 10, 13, and 15 days (d).

FIG. 15A depicts the mRNA levels of GR target gene GILZ in LNCaP and CWR22PC cells post ARV-110 treatment at 0, 6, 10, 13, and 15 days (d).

FIG. 15B depicts the mRNA levels of GR target gene PER1 in LNCaP and CWR22PC cells post ARV-110 treatment at 0, 6, 10, 13, and 15 days (d).

FIG. 15C depicts the mRNA levels of GR target gene KLF9 in LNCaP and CWR22PC cells post ARV-110 treatment at 0, 6, 10, 13, and 15 days (d).

FIG. 15D depicts the mRNA levels of GR target gene SGK1 in LNCaP and CWR22PC cells post ARV-110 treatment at 0, 6, 10, 13, and 15 days (d).

FIG. 16A depicts the mRNA of AR target gene KLK4 in CWR22PC and 22Rv1 cells 48 hours post R1881 (100 pM), DEX (30 nM dexamethasone), ARV-110 (1 µM), and compound 1 (0.5 µM) treatment as indicated.

FIG. 16B depicts the mRNA of GR target gene KLF9 in CWR22PC and 22Rv1 cells 48 hours post R1881 (100 pM), DEX (30 nM dexamethasone), ARV-110 (1 µM), and compound 1 (0.5 µM) treatment as indicated.

FIG. 16C depicts the mRNA of GR and AR target gene FKBP5 in CWR22PC and 22Rv1 cells 48 hours post R1881 (100 pM), DEX (30 nM dexamethasone), ARV-110 (1 µM), and compound 1 (0.5 µM) treatment as indicated.

FIG. 16D depicts the mRNA of GR target gene PER1 in CWR22PC and 22Rv1 cells 48 hours post R1881 (100 pM), DEX (30 nM dexamethasone), ARV-110 (1 µM), and compound 1 (0.5 µM) treatment as indicated.

FIG. 16E depicts the mRNA of GR target gene SGK1 in CWR22PC and 22Rv1 cells 48 hours post R1881 (100 pM), DEX (30 nM dexamethasone), ARV-110 (1 µM), and compound 1 (0.5 µM) treatment as indicated.

FIG. 17A depicts the number of CWR22PC cells under the indicated treatment conditions for 21 days (vehicle, R1881, R1881+ARV-110, R1881+ARV-110+DEX, and R1881+ARV-110+DEX+compound 1).

FIG. 17B depicts the PSA levels in CWR22PC cells under the indicated treatment conditions for 21 days (vehicle, R1881, R1881+ARV-110, R1881+ARV-110+DEX, and R1881+ARV-110+DEX+compound 1). Assays were done in CSS media, R1881: 100 pM synthetic AR ligand; DEX: 30 nM dexamethasone; compound 1: 0.5 µM; ARV-110: 1 µM.

FIG. 18A depicts the mRNA levels of AR target gene KLK3 in CWR22PC cells after 21 days of the indicated treatment conditions (vehicle, R1881, R1881+ARV-110, R1881+ARV-110+DEX, and R1881+ARV-110+DEX+compound 1).

FIG. 18B depicts the mRNA levels of GR target gene GILZ in CWR22PC cells after 21 days of the indicated treatment conditions (vehicle, R1881, R1881+ARV-110, R1881+ARV-110+DEX, and R1881+ARV-110+DEX+compound 1).

FIG. 18C depicts the mRNA levels of GR target gene PER1 in CWR22PC cells after 21 days of the indicated treatment conditions (vehicle, R1881, R1881+ARV-110, R1881+ARV-110+DEX, and R1881+ARV-110+DEX+compound 1).

FIG. 18D depicts the mRNA levels of GR target gene KLF9 in CWR22PC cells after 21 days of the indicated treatment conditions (vehicle, R1881, R1881+ARV-110, R1881+ARV-110+DEX, and R1881+ARV-110+DEX+compound 1).

### DETAILED DESCRIPTION OF THE INVENTION

The Glucocorticoid Receptor (GR) is a member of the nuclear receptor superfamily of ligand-activated transcription factors. GR is activated by its endogenous steroid hormone ligand, cortisol, and by synthetic glucocorticoid drugs such as dexamethasone. Several preclinical studies have established a role for GR in mediating resistance to both targeted therapies and conventional chemotherapies in epithelial cancers. Glucocorticoids have been reported to confer resistance to antimetabolites, taxanes and platinum compounds in lung, prostate, bladder, renal, ovarian and triple negative breast cancers (Skor et al. 2013, Gassler et al. 2005, Li et al. 2017, Zhang et al. 2007). GR has also been reported to confer resistance to antiandrogen therapies in prostate cancer (Arora et al. 2013, Shah et al. 2017, Puhr et al. 2018). Therefore, a molecule that blocks GR activation may have therapeutic potential in patients with solid tumors or prostate cancer.

Prostate cancer is the second leading cause of cancer-related death in men in the US. Androgen deprivation and blockade are commonly used to treat prostate cancer. However, relapse occurs with subsequent progression to metastatic castration-resistant prostate cancer (mCRPC) after treatment with androgen biosynthesis inhibitors or AR antagonists through multiple acquired resistance mechanisms. These resistance mechanisms can be AR-dependent mechanisms including AR amplification, AR point mutations, AR variants or intra-tumoral androgen production, AR-bypass mechanisms such as GR signaling, or AR-independent mechanisms that involve lineage switching or alternate oncogenic pathway activation. New therapeutic strategies are needed for patients who relapse with mCRPC.

PROteolytic-TArgeting-Chimera (PROTAC®) Androgen Receptor (AR) degraders hold the promise to control mCRPC after progressing on standard-of-care antiandrogen therapy, by preventing AR activity through AR protein elimination. PROTAC® degraders harness the human body’s natural E3 ligase-ubiquitin proteasome system for protein degradation. AR degradation may overcome several AR-dependent resistance mechanisms to current antiandrogen therapies that block AR activity, such as loss of AR occupancy through increased production of AR or androgens, or mutations in AR that result in promiscuous AR activation that renders AR antagonists to function as agonists. One example of a PROTAC® AR degrader is ARV-110, which is currently being evaluated in a Phase ½ dose clinical study in patients with mCRPC. Data from the clinical trials has shown that ARV-110 demonstrates targeted degradation of the AR protein in human tissues, with an efficacy signal in a subset of heavily pretreated mCRPC patients. Petrilak at al., (ASCO, 2020) revealed intrinsic resistance to ARV-110 in a subset of patients. One of ordinary skill in the art would understand from these results that prostate cancer in a subject may be successfully treated by administration to the subject a therapeutically effective amount of an AR degrader, including but not limited to ARV-110.

Zhou et al have previously shown that GR is upregulated and drives resistance to enzalutamide in prostate cancer models in response to glucocorticoids, and antagonizing GR with compound 1 restores sensitivity to enzalutamide treatment (Zhou et al., AACR-NCI-EORTC, 27 Oct. 2019) suggesting GR as a potential bypass resistance mechanism to antiandrogen therapy. Disclosed herein is the surprising discovery that the treatment of cancer cells, including prostate cancer cells, with an AR degrader may lead to the cancer cells exhibiting resistance to further treatment with the AR degrader. Also disclosed herein is the surprising discovery that cancer cells treated with an AR degrader may become resistant to further treatment with the AR degrader through upregulation of GR in the cancer cells. Further disclosed herein is the surprising result that cancer cells treated with an AR degrader that exhibit resistance to further treatment with the AR degrader may be treated by administration to the cells a GR antagonist. One of ordinary skill in the art would understand these results that cancer in a subject, including prostate cancer, may be successfully treated by administration to the subject of a therapeutically effective amount of a combination of an AR degrader, including but not limited to ARV-110, in combination with a GR antagonist, including but not limited to compound 1.

### Method of Treatment

Disclosed herein is a method of treating prostate cancer in a subject in need thereof; the method comprising administering a glucocorticoid receptor (GR) antagonist (e.g., compound 1, or a pharmaceutically acceptable salt thereof). In some embodiments, one or more cells comprising the prostate cancer in the subject exhibits an elevated expression of glucocorticoid receptor (GR). In some embodiments, the expression of GR protein levels are measured by GR immunohistochemistry (IHC) assays. In some embodiments, the expression of GR transcript levels are measured using assays such as quantitative polymerase chain reaction (qPCR), microarray, and RNA sequencing, or assays commercially available from companies such as Fluidigm and Nanostring.

Disclosed herein is a method of treating prostate cancer in a subject in need thereof wherein one or more of the cells comprising the prostate cancer exhibits increased expression level of glucocorticoid receptor (GR).

In some embodiments, the GR protein levels are determined by IHC and “increased expression level of glucocorticoid receptor (GR)” is defined as an IHC-derived GR score above a certain threshold. In some embodiments, the score is an H-score ranging from 0 to about 300 (See Cancer Management and Research 2017:9; 65-72, which is hereby incorporated by reference for such disclosures). In some embodiments, the one or more of the cells comprising the prostate cancer exhibits increased expression level of glucocorticoid receptor (GR) when the H-score is about 25, about 50, about 75, about 100, about 125, about 150, about 175, about 200, about 225, about 250, about 275, or about 300. In some embodiments, the one or more of the cells comprising the prostate cancer exhibits increased expression level of glucocorticoid receptor (GR) when the H-score is above about 25, above about 50, above about 75, above about 100, about 125, about 150, about 175, about 200, about 225, about 250, about 275, or about 300.

In some embodiments, “increased expression level of glucocorticoid receptor (GR)” is defined based on the percentage of cells that stain weakly, moderately, or strongly for the glucocorticoid receptor (GR), with the threshold defining the minimal percentage of cells that are required to stain positive for GR at the various intensity levels (≥a% of prostate tumor cells stain weakly for GR, ≥b% of prostate tumor cells stain moderately for GR, ≥c% of prostate tumor cells stain strongly for GR, or a combination thereof). In some embodiments, the one or more of the cells comprising the prostate cancer exhibits increased expression level of glucocorticoid receptor (GR) when ≥ about 10%, ≥ about 15%, ≥ about 20%, ≥ about 25%, ≥ about 30%, ≥ about 35%, ≥ about 40%, ≥ about 45%, ≥ about 50%, ≥ about 55%, ≥ about 60%, ≥ about 65%, ≥ about 70%, ≥ about 75%, ≥ about 80%, ≥ about 85%, ≥ about 90%, or ≥ about 95% of the prostate tumor cells stain weakly for GR; when ≥ about 10%, ≥ about 15%, ≥ about 20%, ≥ about 25%, ≥ about 30%, ≥ about 35%, ≥ about 40%, ≥ about 45%, ≥ about 50%, ≥ about 55%, ≥ about 60%, ≥ about 65%, ≥ about 70%, ≥ about 75%, ≥ about 80%, ≥ about 85%, ≥ about 90%, or ≥ about 95% of the prostate tumor cells stain moderately for GR; when ≥ about 10%, ≥ about 15%, ≥ about 20%, ≥ about 25%, ≥ about 30%, ≥ about 35%, ≥ about 40%, ≥ about 45%, ≥ about 50%, ≥ about 55%, ≥ about 60%, ≥ about 65%, ≥ about 70%, ≥ about 75%, ≥ about 80%, ≥ about 85%, ≥ about 90%, or ≥ about 95% of the prostate tumor cells stain strongly for GR; or any combinations thereof.

In some embodiments, “increased expression level of glucocorticoid receptor (GR)” is based on a predictive response signal (PRS) that was discovered by a molecular analysis of multiple tumor subtypes treated with a GR receptor agonist. The PRS may comprise one of more gene products selected from a group consisting of FKBP Prolyl Isomerase 5 (FKBP5), Period Circadian Regulator 1 (PER1), Kruppel Like Factor 9 (KLF9), TSC22 Domain Family Member 3(TSC22D3), Alkaline Phosphatase, Placental (ALPP), Baculoviral IAP Repeat Containing 3(BIRC3), Keratin 6A (KRT6A), Nebulette (NEBL), Serum Amyloid A1 (SAA1), Serum Amyloid A2 (SAA2), and Sodium Channel Epithelial 1 Subunit Alpha (SCNN1A).

In some embodiments, the presence or an absence, and/or or a level of expression of the one or more gene products is detected in the sample obtained from a subject by analyzing the genetic material in the sample. In some embodiments, the genetic material is obtained from blood, serum, plasma, sweat, hair, tears, urine, and other techniques known by one of skill in the art. In some embodiments the sample comprises circulating tumor RNA (ctRNA). In some embodiments the sample comprises peripheral blood mononuclear cells (PBMCs). In some cases, the genetic material is obtained from a tumor biopsy or liquid biopsy. In some embodiments, a tumor biopsy comprises a formalin-fixed paraffin embedded biopsy, a fresh frozen biopsy, a fresh biopsy, or a frozen biopsy. In some embodiments, a liquid biopsy comprises PBMCs, circulating tumor RNA, plasma cell-free RNA, or circulating tumor cells (CTCs). Tumor biopsies can undergo additional analytic processing for sample dissociation, cell sorting, and enrichment of cell populations of interest.

In some embodiments, the PRS comprises an expression level of the one or more gene products, the expression level deviating from a reference expression level. In some embodiments, the expression levels of the one or more gene products are standardized, such as through a z-score. In some embodiments, the expression levels of the two or more gene products is calculated by averaging the standardized values of the two or more gene products. In some embodiments, the expression level is high, relative to the reference expression level. In some embodiments, the expression level is low, relative to the reference expression level. In some embodiments, the reference expression level is derived from an individual, or a group of individuals, that do not have cancer. In some embodiments, the reference expression level is derived from an individual, or a group of individuals, that have cancer that does not therapeutically respond to the glucocorticoid receptor (GR) antagonist. In some embodiments, the expression level deviates from the reference expression level by at least 20%, 30%, 40%, 50%, 60%, 70%, 80%, 90% or 100%.

In some embodiments, “increased expression level of glucocorticoid receptor (GR)” is based on the expression level of the gene product GR, the expression level deviating from a reference expression level. In some embodiments, the expression level of the gene product is standardized, such as through a z-score. In some embodiments, the expression level is high, relative to the reference expression level. In some embodiments, the expression level is low, relative to the reference expression level. In some embodiments, the reference expression level is derived from an individual, or a group of individuals, that do not have cancer. In some embodiments, the reference expression level is derived from an individual, or a group of individuals, that have cancer that does not therapeutically respond to the glucocorticoid receptor (GR) antagonist. In some embodiments, the expression level deviates from the reference expression level by at least 20%, 30%, 40%, 50%, 60%, 70%, 80%, 90% or 100%.

### Glucocorticoid Receptor (GR) Antagonist and Androgen Receptor (AR) Degrader Combination Administration

Disclosed herein is a method of treating prostate cancer in a subject in need thereof, the method comprising administering to the subject a therapeutically effective amount of (i) a glucocorticoid receptor (GR) antagonist (e.g., compound 1, or a pharmaceutically acceptable salt thereof) and (ii) an androgen receptor (AR) degrader.

In some embodiments, one or more cells comprising the prostate cancer in the subject exhibits an elevated expression of glucocorticoid receptor (GR) prior to the administration of the therapeutically effective amount of the GR antagonist (e.g., compound 1, or a pharmaceutically acceptable salt thereof) and the AR degrader.

In some embodiments, the subject in need thereof has not been previously administered an AR degrader prior to the administration of the therapeutically effective amounts of the GR antagonist (e.g., compound 1, or a pharmaceutically acceptable salt thereof) and the AR degrader.

Also disclosed herein is a method of treating prostate cancer in a subject in need thereof, the method comprising administering to the subject a therapeutically effective amount of (i) compound 1, or a pharmaceutically acceptable salt thereof), and (ii) ARV-110.

In some embodiments, one or more cells comprising the prostate cancer in the subject exhibits an elevated expression of glucocorticoid receptor (GR) prior to the administration of the therapeutically effective amount of compound 1, or a pharmaceutically acceptable salt thereof, and ARV-110.

In some embodiments, the subject in need thereof has not been previously administered ARV-110 prior to the administration of the therapeutically effective amounts of compound 1, or a pharmaceutically acceptable salt thereof, and ARV-110.

In some embodiments, the subject in need thereof has not been previously administered an AR degrader for at least a 1-month period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a 2-month period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a 3-month period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a 4-month period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a 5-month period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a 6-month period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a one-year period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a 18-month period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a two-year period prior to the administration of the therapeutically effective amounts of the GR antagonist (e.g., compound 1, or a pharmaceutically acceptable salt thereof) and the AR degrader.

In some embodiments, the subject in need thereof has been previously administered an AR degrader prior to the administration of the therapeutically effective amounts of the GR antagonist (e.g., compound 1, or a pharmaceutically acceptable salt thereof) and the AR degrader.

In some embodiments, one or more cells comprising the prostate cancer in the subject in need thereof has become resistant to the AR degrader prior to the administration of the therapeutically effective amounts of the GR antagonist (e.g., compound 1, or a pharmaceutically acceptable salt thereof) and the AR degrader.

In some embodiments, the subject in need thereof has been previously administered ARV-110 prior to the administration of the therapeutically effective amounts of compound 1, or a pharmaceutically acceptable salt thereof, and ARV-110.

In some embodiments, one or more cells comprising the prostate cancer in the subject in need thereof has become resistant to ARV-110 prior to the administration of the therapeutically effective amounts of compound 1, or a pharmaceutically acceptable salt thereof, and ARV-110.

In some embodiments, the AR degrader has been administered to the subject for a period of time of between about 1 month and about 2 years prior to the administration of the therapeutically effective amounts of the GR antagonist (e.g., compound 1, or a pharmaceutically acceptable salt thereof) and the AR degrader. In some embodiments, the AR degrader has been administered to the subject for about 1 month prior to the administration of the therapeutically effective amounts of the GR antagonist (e.g., compound 1, or a pharmaceutically acceptable salt thereof) and the AR degrader. In some embodiments, the AR degrader has been administered to the subject for about 2 months prior to the administration of the therapeutically effective amounts of the GR antagonist (e.g., compound 1, or a pharmaceutically acceptable salt thereof) and the AR degrader. In some embodiments, the AR degrader has been administered to the subject for about 3 months prior to the administration of the therapeutically effective amounts of the GR antagonist (e.g., compound 1, or a pharmaceutically acceptable salt thereof) and the AR degrader. In some embodiments, the AR degrader has been administered to the subject for about 4 months prior to the administration of the therapeutically effective amounts of the GR antagonist (e.g., compound 1, or a pharmaceutically acceptable salt thereof) and the AR degrader. In some embodiments, the AR degrader has been administered to the subject for about 5 months prior to the administration of the therapeutically effective amounts of the GR antagonist (e.g., compound 1, or a pharmaceutically acceptable salt thereof) and the AR degrader. In some embodiments, the AR degrader has been administered to the subject for about 6 months prior to the administration of the therapeutically effective amounts of the GR antagonist (e.g., compound 1, or a pharmaceutically acceptable salt thereof) and the AR degrader. In some embodiments, the AR degrader has been administered to the subject for about 1 year prior to the administration of the therapeutically effective amounts of the GR antagonist (e.g., compound 1, or a pharmaceutically acceptable salt thereof) and the AR degrader. In some embodiments, the AR degrader has been administered to the subject for about 18 months prior to the administration of the therapeutically effective amounts of the GR antagonist (e.g., compound 1, or a pharmaceutically acceptable salt thereof) and the AR degrader. In some embodiments, the AR degrader has been administered to the subject for about 2 years prior to the administration of the therapeutically effective amounts of the GR antagonist (e.g., compound 1, or a pharmaceutically acceptable salt thereof) and the AR degrader.

In some embodiments, the AR degrader previously administered and the AR degrader administered in the combination with the GR antagonist (e.g., compound 1, or a pharmaceutically acceptable salt thereof) are the same. In some embodiments, the AR degrader previously administered and the AR degrader administered in the combination with the GR antagonist (e.g., compound 1, or a pharmaceutically acceptable salt thereof) are different.

Also disclosed herein is a glucocorticoid receptor (GR) antagonist for use in combination with an androgen receptor (AR) degrader in a method of treating prostate cancer in a subject. Also disclosed herein is an androgen receptor (AR) degrader for use in combination with a glucocorticoid receptor (GR) antagonist in a method of treating prostate cancer in a subject. In one embodiment, one or more cells comprising the prostate cancer in the subject exhibits an elevated expression of GR prior to the administration of the GR antagonist and the AR degrader. In one embodiment, the subject has not been previously administered an AR degrader prior to the administration of the GR antagonist and the AR degrader. In one embodiment, the subject has not been previously administered an AR degrader for at least a 6-month period prior to the administration of the GR antagonist and the AR degrader. In one embodiment, the subject has not been previously administered an AR degrader for at least a one-year period prior to the administration of the GR antagonist and the AR degrader. In one embodiment, the subject has not been previously administered an AR degrader for at least a two-year period prior to the administration of the GR antagonist and the AR degrader. In one embodiment, the subject has been previously administered a second AR degrader prior to the administration of the GR antagonist and the AR degrader. In one embodiment, one or more cells comprising the prostate cancer in the subject has become resistant to the second AR degrader prior to the administration of the GR antagonist and the AR degrader. In one embodiment, the subject has been administered the second AR degrader for a period of time of between about 1 month and about 2 years prior to the administration of the GR antagonist and the AR degrader. In one embodiment, the AR degrader and the second AR degrader are the same. In one embodiment, the AR degrader and the second AR degrader are different.

In one embodiment, the GR antagonist and the AR degrader are administered to the subject concurrently. In one embodiment, the GR antagonist and the AR degrader are administered to the subject consecutively.

Also disclosed herein is compound 1 for use in combination with an ARV-110 in a method of treating prostate cancer in a subject. Also disclosed herein is ARV-110 for use in combination with compound 1, or a pharmaceutically acceptable salt thereof, in a method of treating prostate cancer in a subject. In one embodiment, one or more cells comprising the prostate cancer in the subject exhibits an elevated expression of GR prior to the administration of compound 1, or a pharmaceutically acceptable salt thereof, and ARV-110. In one embodiment, the subject has not been previously administered ARV-110 prior to the administration of compound 1, or a pharmaceutically acceptable salt thereof, and ARV-110. In one embodiment, the subject has not been previously administered ARV-110 for at least a 6-month period prior to the administration of compound 1, or a pharmaceutically acceptable salt thereof, and ARV-110. In one embodiment, the subject has not been previously administered ARV-110 for at least a one-year period prior to the administration of compound 1, or a pharmaceutically acceptable salt thereof, and ARV-110. In one embodiment, the subject has not been previously administered ARV-110 for at least a two-year period prior to the administration of compound 1, or a pharmaceutically acceptable salt thereof, and ARV-110. In one embodiment, the subject has been previously administered a second AR degrader prior to the administration of compound 1, or a pharmaceutically acceptable salt thereof, and ARV-110. In one embodiment, one or more cells comprising the prostate cancer in the subject has become resistant to the second AR degrader prior to the administration of compound 1, or a pharmaceutically acceptable salt thereof, and ARV-110. In one embodiment, the subject has been administered the second AR degrader for a period of time of between about 1 month and about 2 years prior to the administration of compound 1, or a pharmaceutically acceptable salt thereof, and ARV-110.

In one embodiment, compound 1, or a pharmaceutically acceptable salt thereof, and ARV-110 are administered to the subject concurrently. In one embodiment, compound 1, or a pharmaceutically acceptable salt thereof, and ARV-110 are administered to the subject consecutively.

### Glucocorticoid Receptor (GR) Antagonist and Androgen Receptor (AR) Degrader Combination Administration Following Androgen Receptor (AR) DegraderAdministration

Disclosed herein is a method of treating prostate cancer in a subject in need thereof, wherein the subject has been previously administered chemotherapy comprising a first androgen receptor (AR) degrader, the method comprising administering to the subject a therapeutically effective amount of (i) a glucocorticoid receptor (GR) antagonist (e.g., compound 1, or a pharmaceutically acceptable salt thereof) and (ii) a second androgen receptor (AR) degrader.

In some embodiments, one or more cells comprising the prostate cancer in the subject exhibits an elevated expression of glucocorticoid receptor (GR) prior to the administration of the therapeutically effective amount of the GR antagonist (e.g., compound 1, or a pharmaceutically acceptable salt thereof) and the second AR degrader.

In some embodiments, one or more cells comprising the prostate cancer in the subject has become resistant to the first AR degrader.

Disclosed herein is a method of treating prostate cancer in a subject in need thereof, wherein the subject has been previously administered chemotherapy comprising ARV-110, the method comprising administering to the subject a therapeutically effective amount of compound 1, or a pharmaceutically acceptable salt thereof, and (ii) a second androgen receptor (AR) degrader.

In some embodiments, one or more cells comprising the prostate cancer in the subject exhibits an elevated expression of glucocorticoid receptor (GR) prior to the administration of compound 1, or a pharmaceutically acceptable salt thereof, and the second AR degrader.

In some embodiments, one or more cells comprising the prostate cancer in the subject has become resistant to ARV-110.

In some embodiments, the subject has been previously administered chemotherapy comprising the first AR degrader for a period of time of between about 1 month and about 2 years prior to the administration of the therapeutically effective amounts of the GR antagonist (e.g., compound 1, or a pharmaceutically acceptable salt thereof) and the second AR degrader. In some embodiments, the second AR degrader has been administered to the subject for about 1 month prior to the administration of the therapeutically effective amounts of the GR antagonist (e.g., compound 1, or a pharmaceutically acceptable salt thereof) and the second AR degrader. In some embodiments, the second AR degrader has been administered to the subject for about 2 months prior to the administration of the therapeutically effective amounts of the GR antagonist (e.g., compound 1, or a pharmaceutically acceptable salt thereof) and the second AR degrader. In some embodiments, the second AR degrader has been administered to the subject for about 3 months prior to the administration of the therapeutically effective amounts of the GR antagonist (e.g., compound 1, or a pharmaceutically acceptable salt thereof) and the second AR degrader. In some embodiments, the second AR degrader has been administered to the subject for about 4 months prior to the administration of the therapeutically effective amounts of the GR antagonist (e.g., compound 1, or a pharmaceutically acceptable salt thereof) and the second AR degrader. In some embodiments, the second AR degrader has been administered to the subject for about 5 months prior to the administration of the therapeutically effective amounts of the GR antagonist (e.g., compound 1, or a pharmaceutically acceptable salt thereof) and the second AR degrader. In some embodiments, the second AR degrader has been administered to the subject for about 6 months prior to the administration of the therapeutically effective amounts of the GR antagonist (e.g., compound 1, or a pharmaceutically acceptable salt thereof) and the second AR degrader. In some embodiments, the second AR degrader has been administered to the subject for about 1 year prior to the administration of the therapeutically effective amounts of the GR antagonist (e.g., compound 1, or a pharmaceutically acceptable salt thereof) and the second AR degrader. In some embodiments, the second AR degrader has been administered to the subject for about 18 months prior to the administration of the therapeutically effective amounts of the GR antagonist (e.g., compound 1, or a pharmaceutically acceptable salt thereof) and the second AR degrader. In some embodiments, the second AR degrader has been administered to the subject for about 2 years prior to the administration of the therapeutically effective amounts of the GR antagonist (e.g., compound 1, or a pharmaceutically acceptable salt thereof) and the second AR degrader.

In some embodiments, the subject has been previously administered chemotherapy comprising a first AR degrader within one month prior to the administration of the therapeutically effective amounts of the GR antagonist (e.g., compound 1, or a pharmaceutically acceptable salt thereof) and the second AR degrader.

In some embodiments, the subject has been previously administered chemotherapy comprising a first AR degrader within two months prior to the administration of the therapeutically effective amounts of the GR antagonist (e.g., compound 1, or a pharmaceutically acceptable salt thereof) and the second AR degrader. In some embodiments, the subject has been previously administered chemotherapy comprising a first AR degrader within three months prior to the administration of the therapeutically effective amounts of the GR antagonist (e.g., compound 1, or a pharmaceutically acceptable salt thereof) and the second AR degrader. In some embodiments, the subject has been previously administered chemotherapy comprising a first AR degrader within four months prior to the administration of the therapeutically effective amounts of the GR antagonist (e.g., compound 1, or a pharmaceutically acceptable salt thereof) and the second AR degrader. In some embodiments, the subject has been previously administered chemotherapy comprising a first AR degrader within five months prior to the administration of the therapeutically effective amounts of the GR antagonist (e.g., compound 1, or a pharmaceutically acceptable salt thereof) and the second AR degrader. In some embodiments, the subject has been previously administered chemotherapy comprising the first AR degrader within six months prior to the administration of the therapeutically effective amounts of the GR antagonist (e.g., compound 1, or a pharmaceutically acceptable salt thereof) and the second AR degrader. In some embodiments, the subject has been previously administered chemotherapy comprising the first AR degrader within one year prior to the administration of the therapeutically effective amounts of the GR antagonist (e.g., compound 1, or a pharmaceutically acceptable salt thereof) and the second AR degrader.

In some embodiments, the subject has been previously administered chemotherapy comprising the first AR degrader within one year prior to the administration of the therapeutically effective amounts of the GR antagonist (e.g., compound 1, or a pharmaceutically acceptable salt thereof) and the second AR degrader.

In some embodiments, the first AR degrader and the second AR degrader are the same. In some embodiments, the first AR degrader and the second AR degrader are different.

Also disclosed herein is a glucocorticoid receptor (GR) antagonist for use in combination with a second androgen receptor (AR) degrader in a method of treating prostate cancer in a subject, wherein the subject has been previously administered chemotherapy comprising a first androgen receptor (AR) degrader. Also disclosed herein is a second androgen receptor (AR) degrader for use in combination with a glucocorticoid receptor (GR) antagonist in a method of treating prostate cancer in a subject, wherein the subject has been previously administered chemotherapy comprising a first androgen receptor (AR) degrader. In one embodiment, one or more cells comprising the prostate cancer in the subject exhibits an elevated expression of GR prior to the administration of the GR antagonist and the second AR degrader. In one embodiment, one or more cells comprising the prostate cancer in the subject has become resistant to the first AR degrader. In one embodiment, the subject has been previously administered chemotherapy comprising the first AR degrader for a period of time of between about 1 month and about 2 years prior to the administration of the GR antagonist and the second AR degrader. In one embodiment, the subject has been previously administered chemotherapy comprising a first AR degrader within one month prior to the administration of the GR antagonist and the second AR degrader. In one embodiment, the subject has been previously administered chemotherapy comprising the first AR degrader within one year prior to the administration of the GR antagonist and the second AR degrader. In one embodiment, the first AR degrader and the second AR degrader are the same. In one embodiment, the first AR degrader and the second AR degrader are different. In one embodiment, the GR antagonist and the second AR degrader are administered to the subject concurrently. In one embodiment, the GR antagonist and the second AR degrader are administered to the subject consecutively.

Also disclosed herein is compound 1, or a pharmaceutically acceptable salt thereof, for use in combination with a second androgen receptor (AR) degrader in a method of treating prostate cancer in a subject, wherein the subject has been previously administered chemotherapy comprising ARV-110. Also disclosed herein is a second androgen receptor (AR) degrader for use in combination with compound 1, or a pharmaceutically acceptable salt thereof, in a method of treating prostate cancer in a subject, wherein the subject has been previously administered chemotherapy comprising ARV-110. In one embodiment, one or more cells comprising the prostate cancer in the subject exhibits an elevated expression of GR prior to the administration of compound 1, or a pharmaceutically acceptable salt thereof, and the second AR degrader. In one embodiment, one or more cells comprising the prostate cancer in the subject has become resistant to ARV-110. In one embodiment, the subject has been previously administered chemotherapy comprising ARV-110 for a period of time of between about 1 month and about 2 years prior to the administration of compound 1, or a pharmaceutically acceptable salt thereof, and the second AR degrader. In one embodiment, the subject has been previously administered chemotherapy comprising ARV-110 within one month prior to the administration of compound 1, or a pharmaceutically acceptable salt thereof, and the second AR degrader. In one embodiment, the subject has been previously administered chemotherapy comprising the ARV-110 within one year prior to the administration of compound 1, or a pharmaceutically acceptable salt thereof, and the second AR degrader. In one embodiment, compound 1, or a pharmaceutically acceptable salt thereof, and the second AR degrader are administered to the subject concurrently. In one embodiment, compound 1, or a pharmaceutically acceptable salt thereof, and the second AR degrader are administered to the subject consecutively.

### Glucocorticoid Receptor (GR) Antagonist and Androgen Receptor (AR) Degrader Combination Administration With No Previous Androgen Receptor (AR) Degrader Administration

Disclosed herein is a method of treating prostate cancer in a subject in need thereof, the method comprising administering to the subject a therapeutically effective amount of (i) a glucocorticoid receptor (GR) antagonist (e.g., compound 1, or a pharmaceutically acceptable salt thereof) and (ii) an androgen receptor (AR) degrader, wherein the subject has not been previously administered chemotherapy comprising an androgen receptor (AR) degrader prior to the administration of the therapeutically effective amounts of the GR antagonist (e.g., compound 1, or a pharmaceutically acceptable salt thereof) and the AR degrader.

In some embodiments, one or more cells comprising the prostate cancer in the subject exhibits an elevated expression of glucocorticoid receptor (GR) prior to the administration of the therapeutically effective amount of the GR antagonist (e.g., compound 1, or a pharmaceutically acceptable salt thereof) and the AR degrader.

Disclosed herein is a method of treating prostate cancer in a subject in need thereof, the method comprising administering to the subject a therapeutically effective amount of (i) compound 1, or a pharmaceutically acceptable salt thereof, and (ii) ARV-110, wherein the subject has not been previously administered chemotherapy comprising an androgen receptor (AR) degrader prior to the administration of the therapeutically effective amounts of compound 1, or a pharmaceutically acceptable salt thereof, and ARV-110.

In some embodiments, one or more cells comprising the prostate cancer in the subject exhibits an elevated expression of glucocorticoid receptor (GR) prior to the administration of the therapeutically effective amount of compound 1, or a pharmaceutically acceptable salt thereof, and ARV-110.

In some embodiments, the subject in need thereof has not been previously administered an AR degrader for at least a 1-month period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a 2-month period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a 3-month period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a 4-month period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a 5-month period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a 6-month period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a one-year period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a 18-month period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a two-year period prior to the administration of the therapeutically effective amounts of the GR antagonist (e.g., compound 1, or a pharmaceutically acceptable salt thereof) and the AR degrader.

Also disclosed herein is a glucocorticoid receptor (GR) antagonist for use in combination with an androgen receptor (AR) degrader in a method of treating prostate cancer in a subject, wherein the subject has not been previously administered chemotherapy comprising an androgen receptor (AR) degrader. Also disclosed herein is an androgen receptor (AR) degrader for use in combination with a glucocorticoid receptor (GR) antagonist in a method of treating prostate cancer in a subject, wherein the subject has not been previously administered chemotherapy comprising an androgen receptor (AR) degrader. In one embodiment, one or more cells comprising the prostate cancer in the subject exhibits an elevated expression of glucocorticoid receptor (GR) prior to the administration of the GR antagonist and the AR degrader. In one embodiment, the subject has not been previously administered an AR degrader for at least a 6-month period prior to the administration of the GR antagonist and the AR degrader. In one embodiment, the subject has not been previously administered an AR degrader for at least a one-year period prior to the administration of the GR antagonist and the AR degrader. In one embodiment, the subject has not been previously administered an AR degrader for at least a two-year period prior to the administration of the GR antagonist and the AR degrader. In one embodiment, the GR antagonist and the AR degrader are administered to the subject concurrently. In one embodiment, the GR antagonist and the AR degrader are administered to the subject consecutively.

Also disclosed herein is compound 1, or a pharmaceutically acceptable salt thereof, for use in combination with ARV-110 in a method of treating prostate cancer in a subject, wherein the subject has not been previously administered chemotherapy comprising an androgen receptor (AR) degrader. Also disclosed herein is ARV-110 for use in combination with compound 1, or a pharmaceutically acceptable salt thereof, in a method of treating prostate cancer in a subject, wherein the subject has not been previously administered chemotherapy comprising an androgen receptor (AR) degrader. In one embodiment, one or more cells comprising the prostate cancer in the subject exhibits an elevated expression of glucocorticoid receptor (GR) prior to the administration of ARV-110 and compound 1, or a pharmaceutically acceptable salt thereof. In one embodiment, the subject has not been previously administered an AR degrader for at least a 6-month period prior to the administration of ARV-110 and compound 1, or a pharmaceutically acceptable salt thereof. In one embodiment, the subject has not been previously administered an AR degrader for at least a one-year period prior to the administration of ARV-110 and compound 1, or a pharmaceutically acceptable salt thereof. In one embodiment, the subject has not been previously administered an AR degrader for at least a two-year period prior to the administration of ARV-110 and compound 1, or a pharmaceutically acceptable salt thereof. In one embodiment, the ARV-110 and compound 1, or a pharmaceutically acceptable salt thereof are administered to the subject concurrently. In one embodiment, ARV-110 and compound 1, or a pharmaceutically acceptable salt thereof, are administered to the subject consecutively.

### Glucocorticoid Receptor (GR) Antagonist and Androgen Receptor (AR) Degrader Combination Administration Following Determination of Increased Expression Level of GR

Disclosed herein is a method of treating prostate cancer in a subject in need thereof, the method comprising (a) measuring the expression of glucocorticoid receptor (GR) in one or more of the cells comprising the prostate cancer, and (b) if one or more of the cells comprising the prostate cancer exhibits increased expression level of GR, administering to the subject a therapeutically effective amounts of (i) a glucocorticoid receptor (GR) antagonist (e.g., compound 1, or a pharmaceutically acceptable salt thereof) and (ii) an androgen receptor (AR) degrader.

In some embodiments, the subject in need thereof has not been previously administered an AR degrader prior to the administration of the therapeutically effective amounts of the GR antagonist (e.g., compound 1, or a pharmaceutically acceptable salt thereof) and the AR degrader.

Disclosed herein is a method of treating prostate cancer in a subject in need thereof, the method comprising (a) measuring the expression of glucocorticoid receptor (GR) in one or more of the cells comprising the prostate cancer, and (b) if one or more of the cells comprising the prostate cancer exhibits increased expression level of GR, administering to the subject a therapeutically effective amounts of (i) compound 1, or a pharmaceutically acceptable salt thereof, and (ii) ARV-110.

In some embodiments, the subject in need thereof has not been previously administered ARV-110 prior to the administration of the therapeutically effective amounts of compound 1, or a pharmaceutically acceptable salt thereof, and ARV-110.

In some embodiments, the subject in need thereof has not been previously administered an AR degrader for at least a 1-month period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a 2-month period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a 3-month period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a 4-month period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a 5-month period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a 6-month period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a one-year period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a 18-month period prior to the administration of the therapeutically effective amounts of the GR antagonist (e.g., compound 1, or a pharmaceutically acceptable salt thereof) and the AR degrader. In some embodiments, the subject in need thereof has not been previously administered an AR degrader for at least a two-year period prior to the administration of the therapeutically effective amounts of the GR antagonist (e.g., compound 1, or a pharmaceutically acceptable salt thereof) and the AR degrader.

In some embodiments, the subject in need thereof has been previously administered an AR degrader prior to the administration of the therapeutically effective amounts of the GR antagonist (e.g., compound 1, or a pharmaceutically acceptable salt thereof) and the AR degrader.

In some embodiments, one or more cells comprising the prostate cancer in the subject in need thereof has become resistant to the AR degrader prior to the administration of the therapeutically effective amounts of the GR antagonist (e.g., compound 1, or a pharmaceutically acceptable salt thereof) and the AR degrader.

In some embodiments, the AR degrader has been administered to the subject for a period of time of between about 1 month and about 2 years prior to the administration of the therapeutically effective amounts of the GR antagonist (e.g., compound 1, or a pharmaceutically acceptable salt thereof) and the AR degrader. In some embodiments, the AR degrader has been administered to the subject for about 1 month prior to the administration of the therapeutically effective amounts of the GR antagonist (e.g., compound 1, or a pharmaceutically acceptable salt thereof) and the AR degrader. In some embodiments, the AR degrader has been administered to the subject for about 2 months prior to the administration of the therapeutically effective amounts of the GR antagonist (e.g., compound 1, or a pharmaceutically acceptable salt thereof) and the AR degrader. In some embodiments, the AR degrader has been administered to the subject for about 3 months prior to the administration of the therapeutically effective amounts of the GR antagonist (e.g., compound 1, or a pharmaceutically acceptable salt thereof) and the AR degrader. In some embodiments, the AR degrader has been administered to the subject for about 4 months prior to the administration of the therapeutically effective amounts of the GR antagonist (e.g., compound 1, or a pharmaceutically acceptable salt thereof) and the AR degrader. In some embodiments, the AR degrader has been administered to the subject for about 5 months prior to the administration of the therapeutically effective amounts of the GR antagonist (e.g., compound 1, or a pharmaceutically acceptable salt thereof) and the AR degrader. In some embodiments, the AR degrader has been administered to the subject for about 6 months prior to the administration of the therapeutically effective amounts of the GR antagonist (e.g., compound 1, or a pharmaceutically acceptable salt thereof) and the AR degrader. In some embodiments, the AR degrader has been administered to the subject for about 1 year prior to the administration of the therapeutically effective amounts of the GR antagonist (e.g., compound 1, or a pharmaceutically acceptable salt thereof) and the AR degrader. In some embodiments, the AR degrader has been administered to the subject for about 18 months prior to the administration of the therapeutically effective amounts of the GR antagonist (e.g., compound 1, or a pharmaceutically acceptable salt thereof) and the AR degrader. In some embodiments, the AR degrader has been administered to the subject for about 2 years prior to the administration of the therapeutically effective amounts of the GR antagonist (e.g., compound 1, or a pharmaceutically acceptable salt thereof) and the AR degrader.

In some embodiments, the AR degrader previously administered and the AR degrader administered in the combination with the GR antagonist (e.g., compound 1, or a pharmaceutically acceptable salt thereof) are the same. In some embodiments, the AR degrader previously administered and the AR degrader administered in the combination with the GR antagonist (e.g., compound 1, or a pharmaceutically acceptable salt thereof) are different.

Also disclosed herein is a glucocorticoid receptor (GR) antagonist for use in combination with an androgen receptor (AR) degrader in a method of treating prostate cancer in a subject, wherein one or more of the cells comprising the prostate cancer exhibits increased expression level of GR. Also disclosed herein is an androgen receptor (AR) degrader for use in combination with a glucocorticoid receptor (GR) antagonist in a method of treating prostate cancer in a subject, wherein one or more of the cells comprising the prostate cancer exhibits increased expression level of GR. In one embodiment, the subject has not been previously administered an AR degrader prior to the administration of the GR antagonist and the AR degrader. In one embodiment, the subject has not been previously administered an AR degrader for at least a 6-month period prior to the administration of the GR antagonist and the AR degrader. In one embodiment, the subject has not been previously administered an AR degrader for at least a one-year period prior to the administration of the GR antagonist and the AR degrader. In one embodiment, the subject has not been previously administered an AR degrader for at least a two-year period prior to the administration of the GR antagonist and the AR degrader. In one embodiment, the subject has been previously administered a second AR degrader prior to the administration of the GR antagonist and the AR degrader. In one embodiment, one or more cells comprising the prostate cancer in the subject has become resistant to the second AR degrader prior to the administration of the GR antagonist and the AR degrader. In one embodiment, the subject has been administered the second AR degrader for a period of time of between about 1 month and about 2 years prior to the administration of the GR antagonist and the AR degrader. In one embodiment, the AR degrader and the second AR degrader are the same. In one embodiment, the AR degrader and the second AR degrader are different. In one embodiment, the GR antagonist and the AR degrader are administered to the subject concurrently. In one embodiment, the GR antagonist and the AR degrader are administered to the subject consecutively.

Also disclosed herein is compound 1, or a pharmaceutically acceptable salt thereof, for use in combination with ARV-110 in a method of treating prostate cancer in a subject, wherein one or more of the cells comprising the prostate cancer exhibits increased expression level of GR. Also disclosed herein is ARV-110 for use in combination with compound 1, or a pharmaceutically acceptable salt thereof, in a method of treating prostate cancer in a subject, wherein one or more of the cells comprising the prostate cancer exhibits increased expression level of GR. In one embodiment, the subject has not been previously administered ARV-110 prior to the administration of compound 1, or a pharmaceutically acceptable salt thereof, and ARV-110. In one embodiment, the subject has not been previously administered ARV-110 for at least a 6-month period prior to the administration of compound 1, or a pharmaceutically acceptable salt thereof, and ARV-110. In one embodiment, the subject has not been previously administered ARV-110 for at least a one-year period prior to the administration of compound 1, or a pharmaceutically acceptable salt thereof, and ARV-110. In one embodiment, the subject has not been previously administered ARV-110 for at least a two-year period prior to the administration of compound 1, or a pharmaceutically acceptable salt thereof, and ARV-110. In one embodiment, the subject has been previously administered a second AR degrader prior to the administration of compound 1, or a pharmaceutically acceptable salt thereof, and ARV-110. In one embodiment, one or more cells comprising the prostate cancer in the subject has become resistant to ARV-110 prior to the administration of compound 1, or a pharmaceutically acceptable salt thereof, and ARV-110. In one embodiment, the subject has been administered the second AR degrader for a period of time of between about 1 month and about 2 years prior to the administration of compound 1, or a pharmaceutically acceptable salt thereof, and ARV-110. In one embodiment, compound 1, or a pharmaceutically acceptable salt thereof, and ARV-110 are administered to the subject concurrently. In one embodiment, compound 1, or a pharmaceutically acceptable salt thereof, and ARV-110 are administered to the subject consecutively.

### Glucocorticoid Receptor (GR) Antagonist Administration Following AR Degrader Administration and Determination of Increased Expression Level of GR

Disclosed herein is a method of treating prostate cancer in a subject in need thereof, the method comprising (a) administering to the subject an androgen antagonist (AR) degrader for a period of time, (b) measuring the expression of glucocorticoid receptor (GR) in one or more of the cells comprising the prostate cancer following administration of the AR degrader for the period of time, and (c) if one or more of the cells comprising the prostate cancer exhibits an increased expression level of GR following administration of the AR degrader for a period of time, administering to the subject a therapeutically effective amount of a glucocorticoid receptor (GR) antagonist (e.g., compound 1, or a pharmaceutically acceptable salt thereof).

Disclosed herein is a method of treating prostate cancer in a subject in need thereof, the method comprising (a) administering to the subject ARV-110 for a period of time, (b) measuring the expression of glucocorticoid receptor (GR) in one or more of the cells comprising the prostate cancer following administration of ARV-110 for the period of time, and (c) if one or more of the cells comprising the prostate cancer exhibits an increased expression level of GR following administration of ARV-110 for a period of time, administering to the subject a therapeutically effective amount of compound 1, or a pharmaceutically acceptable salt thereof.

In some embodiments, the period of time is between about 1 month and about 2 years. In some embodiments, the period of time is about one month. In some embodiments, the period of time is about two months. In some embodiments, the period of time is about 3 months. In some embodiments, the period of time is about four months. In some embodiments, the period of time is about five months. In some embodiments, the period of time is about six months. In some embodiments, the period of time is about one year. In some embodiments, the period of time is about 18 months. In some embodiments, the period of time is about two years.

In some embodiments, the method further comprises administering to the subject a therapeutically effective amount of an AR degrader in combination with the therapeutically effective amount of a GR antagonist (e.g., compound 1, or a pharmaceutically acceptable salt thereof). In some embodiments, the AR degrader previously administered and the AR degrader administered in the combination with the GR antagonist (e.g., compound 1, or a pharmaceutically acceptable salt thereof) are the same. In some embodiments, the AR degrader previously administered and the AR degrader administered in the combination with the GR antagonist (e.g., compound 1, or a pharmaceutically acceptable salt thereof) are different.

Also disclosed herein is a glucocorticoid receptor (GR) antagonist for use in combination with an androgen receptor (AR) degrader in a method of treating prostate cancer in a subject, wherein after administration of the AR degrader for a period of time, one or more of the cells comprising the prostate cancer exhibits increased expression level of GR. Also disclosed herein is an androgen receptor (AR) degrader for use in combination with a glucocorticoid receptor (GR) antagonist in a method of treating prostate cancer in a subject, wherein after administration of the AR degrader for a period of time, one or more of the cells comprising the prostate cancer exhibits increased expression level of GR. In one embodiment, the period of time is between about 1 month and about 2 years. In one embodiment, the AR degrader and the second AR degrader are the same. In one embodiment, the AR degrader and the second AR degrader are different. In one embodiment, the GR antagonist and the AR degrader are administered to the subject concurrently. In one embodiment, the GR antagonist and the AR degrader are administered to the subject consecutively.

Also disclosed herein is compound 1, or a pharmaceutically acceptable salt thereof, for use in combination with ARV-110 in a method of treating prostate cancer in a subject, wherein after administration of ARV-110 for a period of time, one or more of the cells comprising the prostate cancer exhibits increased expression level of GR. Also disclosed herein is ARV-110 for use in combination compound 1, or a pharmaceutically acceptable salt thereof, in a method of treating prostate cancer in a subject, wherein after administration of ARV-110 for a period of time, one or more of the cells comprising the prostate cancer exhibits increased expression level of GR. In one embodiment, compound 1, or a pharmaceutically acceptable salt thereof, and ARV-110 are administered to the subject concurrently. In one embodiment, compound 1, or a pharmaceutically acceptable salt thereof, and ARV-110 are administered to the subject consecutively.

### Glucocorticoid Receptor (GR) Antagonist and Androgen Receptor (AR) Degrader Combination Administration With Previous Androgen Receptor (AR) Antagonist and Glucocorticoid Receptor (GR) Antagonist Administration

Also disclosed herein is a method of treating prostate cancer in a subject in need thereof, the method comprising administering to the subject a therapeutically effective amount of (i) a second glucocorticoid receptor (GR) antagonist and (ii) an androgen receptor (AR) degrader, wherein the subject has been previously administered chemotherapy comprising (a) a first glucocorticoid receptor (GR) antagonist and (b) an androgen receptor (AR) inhibitor prior to the administration of the therapeutically effective amounts of the second GR antagonist and the AR degrader.

In some embodiments, the AR inhibitor is 3,3′-diindolylmethane (DIM), abiraterone acetate, bexlosteride, bicalutamide, dutasteride, epristeride, enzalutamide, apalutamide, finasteride, flutamide, izonsteride, ketoconazole, N-butylbenzene-sulfonamide, nilutamide, megestrol, steroidal antiandrogens, and turosteride, or any combinations thereof. In some embodiments, the AR inhibitor is enzalutamide.

In some embodiments, the chemotherapy comprising the first GR antagonist and the AR inhibitor has been administered to the subject for a period of time of between about 1 month and about 2 years prior to the administration of the therapeutically effective amounts of the second GR antagonist and the AR degrader.

In some embodiments, the chemotherapy comprising the first GR antagonist and the AR inhibitor has been administered to the subject for about 1 month prior to the administration of the therapeutically effective amounts of the second GR antagonist and the AR degrader. In some embodiments, the chemotherapy comprising the first GR antagonist and the AR inhibitor has been administered to the subject for about 2 months prior to the administration of the therapeutically effective amounts of the second GR antagonist and the AR degrader. In some embodiments, the chemotherapy comprising the first GR antagonist and the AR inhibitor has been administered to the subject for about 3 months prior to the administration of the therapeutically effective amounts of the second GR antagonist and the AR degrader. In some embodiments, the chemotherapy comprising the first GR antagonist and the AR inhibitor has been administered to the subject for about 4 months prior to the administration of the therapeutically effective amounts of the second GR antagonist and the AR degrader. In some embodiments, the chemotherapy comprising the first GR antagonist and the AR inhibitor has been administered to the subject for about 5 months prior to the administration of the therapeutically effective amounts of the second GR antagonist and the AR degrader. In some embodiments, the chemotherapy comprising the first GR antagonist and the AR inhibitor has been administered to the subject for about 6 months prior to the administration of the therapeutically effective amounts of the second GR antagonist and the AR degrader. In some embodiments, the chemotherapy comprising the first GR antagonist and the AR inhibitor has been administered to the subject for about 1 year prior to the administration of the therapeutically effective amounts of the second GR antagonist and the AR degrader. In some embodiments, the chemotherapy comprising the first GR antagonist and the AR inhibitor has been administered to the subject for about 18 months prior to the administration of the therapeutically effective amounts of the second GR antagonist and the AR degrader. In some embodiments, the chemotherapy comprising the first GR antagonist and the AR inhibitor has been administered to the subject for about 2 years prior to the administration of the therapeutically effective amounts of the second GR antagonist and the AR degrader.

In some embodiments, one or more cells comprising the prostate cancer in the subject in need thereof has become resistant to the AR inhibitor prior to the administration of the therapeutically effective amounts of the second GR antagonist and the AR degrader.

In some embodiments, the second GR antagonist and AR degrader are administered once the one or more cells comprising the prostate cancer in the subject in need thereof has become resistant to the AR inhibitor.

In some embodiments, the first GR antagonist and the second GR antagonists are the same. In some embodiments, the first GR antagonist and the second GR antagonists are different.

Also disclosed herein is a second glucocorticoid receptor (GR) antagonist for use in combination with an androgen receptor (AR) degrader in a method of treating prostate cancer in a subject, wherein the subject has been previously administered chemotherapy comprising a first glucocorticoid receptor (GR) antagonist and an androgen receptor (AR) inhibitor. Also disclosed herein is an androgen receptor (AR) degrader for use in combination with a second glucocorticoid receptor (GR) antagonist in a method of treating prostate cancer in a subject, wherein the subject has been previously administered chemotherapy comprising a first glucocorticoid receptor (GR) antagonist and an androgen receptor (AR) inhibitor.

In one embodiment, the AR inhibitor is 3,3′-diindolylmethane (DIM), abiraterone acetate, bexlosteride, bicalutamide, dutasteride, epristeride, enzalutamide, apalutamide, finasteride, flutamide, izonsteride, ketoconazole, N-butylbenzene-sulfonamide, nilutamide, megestrol, steroidal antiandrogens, and turosteride, or any combinations thereof. In one embodiment, the AR inhibitor is enzalutamide.

In one embodiment, one or more cells comprising the prostate cancer in the subject exhibits an elevated expression of GR prior to the administration of the second GR antagonist and the AR degrader. In one embodiment, one or more cells comprising the prostate cancer in the subject has become resistant to the AR inhibitor. In one embodiment, the subject has been previously administered chemotherapy comprising the first GR antagonist and the AR inhibitor for a period of time of between about 1 month and about 2 years prior to the administration of the second GR antagonist and the AR degrader. In one embodiment, the first GR antagonist and the second GR antagonist are the same. In one embodiment, the first GR antagonist and the second GR antagonist are different. In one embodiment, the second GR antagonist and the AR degrader are administered to the subject concurrently. In one embodiment, the second GR antagonist and the AR degrader are administered to the subject consecutively.

### Androgen Receptor (AR) Degrader Administration With Previous Androgen Receptor (AR) Antagonist and Glucocorticoid Receptor (GR) Antagonist Administration

Also disclosed herein is a method of treating prostate cancer in a subject in need thereof, the method comprising administering to the subject a therapeutically effective amount of an androgen receptor (AR) degrader, wherein the subject has been previously administered chemotherapy comprising (a) a glucocorticoid receptor (GR) antagonist and (b) an androgen receptor (AR) inhibitor prior to the administration of the therapeutically effective amounts of the AR degrader.

In some embodiments, the method further comprises administering a glucocorticoid receptor (GR) antagonist in addition to the AR degrader.

Also disclosed herein is a method of treating prostate cancer in a subject in need thereof, the method comprising administering to the subject a therapeutically effective amount ARV-110, wherein the subject has been previously administered chemotherapy comprising (a) a glucocorticoid receptor (GR) antagonist and (b) an androgen receptor (AR) inhibitor prior to the administration of the therapeutically effective amounts of the AR degrader. In some embodiments are provided a method of treating prostate cancer in a subject in need thereof, the method comprising administering to the subject a therapeutically effective amount of an ARV-110, wherein the subject has been previously administered chemotherapy comprising (a) compound 1, or a pharmaceutically acceptable salt thereof, and (b) an androgen receptor (AR) inhibitor prior to the administration of the therapeutically effective amounts of ARV-110.

In some embodiments, the AR inhibitor is 3,3′-diindolylmethane (DIM), abiraterone acetate, bexlosteride, bicalutamide, dutasteride, epristeride, enzalutamide, apalutamide, finasteride, flutamide, izonsteride, ketoconazole, N-butylbenzene-sulfonamide, nilutamide, megestrol, steroidal antiandrogens, and turosteride, or any combinations thereof. In some embodiments, the AR inhibitor is enzalutamide.

In some embodiments, the chemotherapy comprising the GR antagonist and the AR inhibitor has been administered to the subject for a period of time of between about 1 month and about 2 years prior to the administration of the therapeutically effective amounts of the AR degrader.

In some embodiments, the chemotherapy comprising the GR antagonist and the AR inhibitor has been administered to the subject for about 1 month prior to the administration of the therapeutically effective amounts of the AR degrader. In some embodiments, the chemotherapy comprising the GR antagonist and the AR inhibitor has been administered to the subject for about 2 months prior to the administration of the therapeutically effective amounts of the AR degrader. In some embodiments, the chemotherapy comprising the GR antagonist and the AR inhibitor has been administered to the subject for about 3 months prior to the administration of the therapeutically effective amounts of the AR degrader. In some embodiments, the chemotherapy comprising the GR antagonist and the AR inhibitor has been administered to the subject for about 4 months prior to the administration of the therapeutically effective amounts of the AR degrader. In some embodiments, the chemotherapy comprising the GR antagonist and the AR inhibitor has been administered to the subject for about 5 months prior to the administration of the therapeutically effective amounts of the AR degrader. In some embodiments, the chemotherapy comprising the GR antagonist and the AR inhibitor has been administered to the subject for about 6 months prior to the administration of the therapeutically effective amounts of the AR degrader. In some embodiments, the chemotherapy comprising the GR antagonist and the AR inhibitor has been administered to the subject for about 1 year prior to the administration of the therapeutically effective amounts of the AR degrader. In some embodiments, the chemotherapy comprising the GR antagonist and the AR inhibitor has been administered to the subject for about 18 months prior to the administration of the therapeutically effective amounts of the AR degrader. In some embodiments, the chemotherapy comprising the GR antagonist and the AR inhibitor has been administered to the subject for about 2 years prior to the administration of the therapeutically effective amounts of the AR degrader.

In some embodiments, one or more cells comprising the prostate cancer in the subject in need thereof has become resistant to the AR inhibitor prior to the administration of the therapeutically effective amounts of the AR degrader.

In some embodiments, the AR degrader is administered once the one or more cells comprising the prostate cancer in the subject in need thereof has become resistant to the AR inhibitor.

Also disclosed herein is an androgen receptor (AR) degrader for use in a method of treating prostate cancer in a subject, wherein the subject has been previously administered chemotherapy comprising a glucocorticoid receptor (GR) antagonist and an androgen receptor (AR) inhibitor.

Also disclosed herein is ARV-110 for use in a method of treating prostate cancer in a subject, wherein the subject has been previously administered chemotherapy comprising compound 1, or a pharmaceutically acceptable salt thereof, and an androgen receptor (AR) inhibitor.

In one embodiment, the AR inhibitor is 3,3′-diindolylmethane (DIM), abiraterone acetate, bexlosteride, bicalutamide, dutasteride, epristeride, enzalutamide, apalutamide, finasteride, flutamide, izonsteride, ketoconazole, N-butylbenzene-sulfonamide, nilutamide, megestrol, steroidal antiandrogens, and turosteride, or any combinations thereof. In one embodiment, the AR inhibitor is enzalutamide.

In one embodiment, one or more cells comprising the prostate cancer in the subject exhibits an elevated expression of GR prior to the administration of the AR degrader. In one embodiment, one or more cells comprising the prostate cancer in the subject has become resistant to the AR inhibitor. In one embodiment, the subject has been previously administered chemotherapy comprising the GR antagonist and the AR inhibitor for a period of time of between about 1 month and about 2 years prior to the administration of the second GR antagonist and the AR degrader. In one embodiment, the second GR antagonist and the AR degrader are administered to the subject concurrently. In one embodiment, the second GR antagonist and the AR degrader are administered to the subject consecutively.

### Prostate Cancer

Prostate cancer is the second most common cause of cancer death in men in the United States, and approximately one in every six American men will be diagnosed with the disease during his lifetime. Treatment aimed at eradicating the tumor is unsuccessful in 30% of men.

In some embodiments, the prostate cancer is chemoresistant cancer, radio resistant cancer, antiandrogen resistant, or refractory cancer. In some embodiments, the prostate cancer is relapsed cancer, persistent cancer, or recurrent cancer.

In some embodiments, the prostate cancer is metastatic prostate cancer. In some embodiments, the prostate cancer is metastatic castration-resistant prostate cancer. In some embodiments, the prostate cancer is castration-resistant prostate cancer (CRPC). In some embodiments, the prostate cancer is castration-sensitive prostate cancer.

In some embodiments, the prostate cancer is acinar adenocarcinoma, atrophic carcinoma, foamy carcinoma, colloid carcinoma, or signet ring carcinoma. In some embodiments, the prostate cancer is ductal adenocarcinoma, transitional cell cancer, urothelial cancer, squamous cell cancer, carcinoid cancer, small cell cancer, sarcoma cancer, or sarcomatoid cancer. In some embodiments, the prostate cancer is metastatic castration-resistant prostate cancer, doubly-resistant prostate cancer, castration-resistant prostate cancer, hormone-resistant prostate cancer, androgen-independent, or androgen-refractory cancer.

In some instances, antiandrogens are useful for the treatment of prostate cancer during its early stages. In some instances, prostate cancer cells depend on androgen receptor (AR) for their proliferation and survival. Some prostate cancer patients are physically castrated or chemically castrated by treatment with agents that block production of testosterone (e.g. GnRH agonists), alone or in combination with antiandrogens, which antagonize effects of any residual testosterone.

In some instances, prostate cancer advances to a hormone-refractory state in which the disease progresses despite continued androgen ablation or antiandrogen therapy. The hormone-refractory state to which most patients eventually progresses in the presence of continued androgen ablation or antiandrogen therapy is known as “castration resistant” prostate cancer (CRPC). CRPC is associated with an overexpression of AR. AR is expressed in most prostate cancer cells and overexpression of AR is necessary and sufficient for androgen-independent growth of prostate cancer cells. Failure in hormonal therapy, resulting from development of androgen-independent growth, is an obstacle for successful management of advanced prostate cancer.

While a small minority of CRPC does bypass the requirement for AR signaling, the vast majority of CRPC, though frequently termed “androgen independent prostate cancer” or “hormone refractory prostate cancer,” retains its lineage dependence on AR signaling.

Recently approved therapies that target androgen receptor (AR) signaling such as abiraterone and enzalutamide have been utilized for treating CRPC. Despite these successes, sustained response with these agents is limited by acquired resistance which typically develops within 6-12 months. Doubly resistant prostate cancer is characterized in that tumor cells have become castration resistant and overexpress AR, a hallmark of CRPC. However, cells remain resistant when treated with second generation antiandrogens. Doubly resistant prostate cancer cells are characterized by a lack of effectiveness of second generation antiandrogens in inhibiting tumor growth.

Resistant prostate cancer (e.g., doubly resistant and castration resistant prostate cancers) occurs when cancer cells overexpress androgen receptors (AR). AR target gene expression is inhibited when the cells are treated with a second generation antiandrogen. In some instances, increased signaling through the glucocorticoid receptor (GR) compensates for inhibition of androgen receptor signaling in resistant prostate cancer. Double resistant prostate cancer develops when expression of a subset of those AR target genes is restored. In some instances, GR activation is responsible for this target gene activation. In some embodiments, GR transcription is activated in patients susceptible to or suffering from resistant prostate cancer (e.g., doubly resistant and castration resistant prostate cancers). In some instances, GR upregulation in cancer cells confers resistance to antiandrogens.

In some embodiments, the prostate cancer is resistant to the AR degrader. In some embodiments, the prostate cancer is resistant to the AR inhibitor.

Some embodiments provided herein describe the use of the GR antagonist (e.g., compound 1, or a pharmaceutically acceptable salt thereof) for treating prostate cancer in a subject in need thereof, including doubly resistant prostate cancer and castration resistant prostate cancer. In some embodiments, the subject in need has elevated tumor GR expression. In some embodiments, the GR inhibitor is also an AR signaling inhibitor or antiandrogen.

In some embodiments, the prostate cancer in the subject is metastatic prostate cancer. In some embodiments, the prostate cancer in the subject is metastatic castration-resistant prostate cancer. In some embodiments, the prostate cancer in the subject is Localized high risk prostate cancer, recurrent prostate cancer, non-metastatic CRPC (nmCRPC), non-metastatic castration-sensitive prostate cancer, or metastatic castration-sensitive prostate cancer.

### Further Combination

In some embodiments, the GR antagonist (e.g., compound 1, or a pharmaceutically acceptable salt thereof) and the AR degrader are used in combination with one or more additional therapeutic agents.

In some embodiments, the one or more additional therapeutic agents is selected from one or more androgen receptor inhibitors, one or more chemotherapeutic agents, and one or more immunotherapy agents, or any combinations thereof. In some embodiments, the one or more androgen receptor inhibitors is selected from 3,3′-diindolylmethane (DIM), abiraterone acetate, bexlosteride, bicalutamide, dutasteride, epristeride, enzalutamide, apalutamide, finasteride, flutamide, izonsteride, ketoconazole, N-butylbenzene-sulfonamide, nilutamide, megestrol, steroidal antiandrogens, and turosteride, or any combinations thereof.

In some embodiments, the one or more additional therapeutic agents is an AR signaling inhibitor or antiandrogen. In certain embodiments, the AR signaling inhibitor is an AR antagonist. In some embodiments, the second or additional therapeutic agent is selected from finasteride, dutasteride, alfatradiol, cyproterone acetate, spironolactone, danazol, gestrinone, ketoconazole, abiraterone acetate, enzalutamide, apalutamide, darolutamide, danazol, gestrinone, danazol, simvastatin, aminoglutethimide, atorvastatin, simvastatin, progesterone, cyproterone acetate, medroxyprogesterone acetate, megestrol acetate, chlormadinone acetate, spironolactone, drospirenone, estradiol, ethinyl estradiol, diethylstilbestrol, conjugated equine estrogens, buserelin, deslorelin, gonadorelin, goserelin, histrelin, leuprorelin, nafarelin, triptorelin, abarelix, cetrorelix, degarelix, ganirelix, or any combinations or any salts thereof. In some embodiments, the second or additional therapeutic agent is selected from flutamide, nilutamide, bicalutamide, enzalutamide, apalutamide, darolutamide, cyproterone acetate, megestrol acetate, chlormadinone acetate, spironolactone, canrenone, drospirenone, ketoconazole, topilutamide, cimetidine, or any combinations or any salts thereof. In some embodiments, the AR signaling inhibitor is 3,3′-diindolylmethane (DIM), abiraterone acetate, apalutamide, darolutamide, bexlosteride, bicalutamide, dutasteride, epristeride, enzalutamide, finasteride, flutamide, izonsteride, ketoconazole, N-butylbenzene-sulfonamide, nilutamide, megestrol, steroidal antiandrogens, turosteride, or any combinations thereof. In some embodiments, the AR signaling inhibitor is flutamide, nilutamide, bicalutamide, or megestrol. In some embodiments, the AR signaling inhibitor is apalutamide. In other embodiments, the AR signaling inhibitor is enzalutamide.

In some embodiments, the anti-cancer agent is mitoxantrone, estramustine, etoposide, vinblastine, carboplatin, vinorelbine, paclitaxel, daunomycin, darubicin, epirubicin, docetaxel, cabazitaxel, or doxorubicin. In some embodiments, the anti-cancer agent is paclitaxel, daunomycin, darubicin, epirubicin, docetaxel, cabazitaxel, or doxorubicin. In certain embodiments, the anti-cancer agent is docetaxel.

### Glucocorticoid Receptor (GR) Antagonists

Disclosed herein are methods of treating prostate cancer, wherein the glucocorticoid receptor (GR) antagonist is a selective GR antagonist. Disclosed herein are methods for treating prostate cancer wherein the glucocorticoid receptor (GR) antagonist is an unselective GR antagonist. In some embodiments, the GR antagonist is mifepristone, cyproterone acetate, relacorilant (CORT125134), exicorilant (CORT125281), miricorilant (CORT118335), CORT113176, CORT108297, PT150 (formerly Org34517), PT157, or PT162.

In some embodiments, the GR antagonist is a compound of Formula (I), or a pharmaceutically acceptable salt thereof:

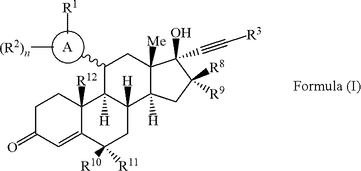

wherein
ring A is a heteroaryl or aryl;
R¹ is -NR^{4a}R^{5a};
each R² is independently -NR⁴R⁵, halo, -OR⁶, —OH, optionally substituted alkyl, or haloalkyl;
R³ is optionally substituted C₂₋₈ alkyl, halo, haloalkyl, optionally substituted cycloalkyl, optionally substituted cycloalkylalkyl, optionally substituted heterocycloalkyl, optionally substituted heterocycloalkylalkyl, optionally substituted heteroalkyl, optionally substituted aryl, optionally substituted heteroaryl, -Si(R⁶)₃, -OR⁶, or -S(O)₂R⁷;
R^{4a} is C₂₋₈ alkyl, optionally substituted cycloalkyl, optionally substituted aryl, optionally substituted heterocycloalkyl, or optionally substituted heteroaryl;
R^{5a} is —H, optionally substituted alkyl, or haloalkyl;
or R^{4a} and R^{5a} are taken together with the N atom to which they are attached to form an optionally substituted heterocycloalkyl;
R⁴ and R⁵ are each independently —H, optionally substituted alkyl, or haloalkyl;
or R⁴ and R⁵ are taken together with the N atom to which they are attached to form an optionally substituted heterocycloalkyl;
each R⁶ is independently optionally substituted alkyl or haloalkyl;
R⁷ is optionally substituted alkyl or haloalkyl;
R⁸ and R⁹ are each independently —H, optionally substituted alkyl, haloalkyl, or halo;
R¹⁰ and R¹¹ are each independently —H, optionally substituted alkyl, halo, or haloalkyl;
R¹² is hydrogen, optionally substituted alkyl, haloalkyl, hydroxy, or halo;
n is 0, 1, or 2.

In some embodiments of compounds of Formula (I), R¹² is C₁₋₆ alkyl or hydrogen. In some embodiments of compounds of Formula (I), R¹² is methyl. In some embodiments of compounds of Formula (I), R¹² is H. In some embodiments of compounds of Formula (I), ring A is phenyl. In some embodiments of compounds of Formula (I), R^{4a} is C₂₋₈ alkyl. In some embodiments of compounds of Formula (I), R^{4a} is C₃₋₆ alkyl. In some embodiments of compounds of Formula (I), R^{4a} is C₂₋₄ alkyl. In some embodiments of compounds of Formula (I), R^{4a} is ethyl, i-propyl, or t-butyl. In some embodiments of compounds of Formula (I), R^{5a} is —H, optionally substituted alkyl, or haloalkyl. In some embodiments of compounds of Formula (I), R^{5a} is —H or alkyl. In some embodiments of compounds of Formula (I), R^{5a} is C₁₋₆ alkyl. In some embodiments of compounds of Formula (I), n is 0 or 1. In some embodiments of compounds of Formula (I), each R² is independently halo. In some embodiments of compounds of Formula (I), R³ is optionally substituted C₂₋₈ alkyl, haloalkyl, or optionally substituted cycloalkyl. In some embodiments of compounds of Formula (I), R³ is C₄₋₈ alkyl. In some embodiments of compounds of Formula (I), R⁸ and R⁹ are —H. In some embodiments of compounds of Formula (I), R¹⁰ and R¹¹ are each —H.

In some embodiments of compounds of Formula (I), the compound has the structure of Formula (Ia):

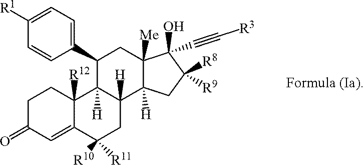

In some embodiments, the GR antagonist is compound 1:

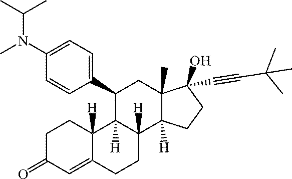

or a pharmaceutically acceptable salt thereof.

In some embodiments, the GR antagonist is compound 1:

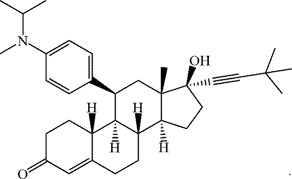

In some embodiments, the GR antagonist is:

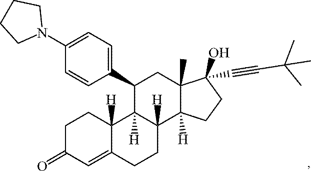

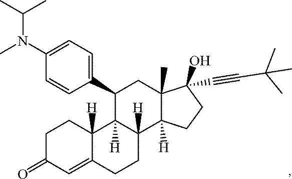

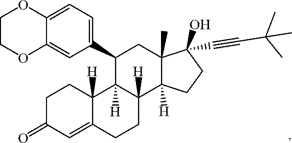

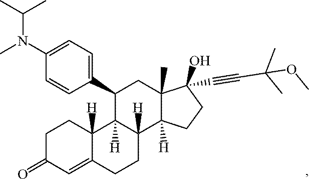

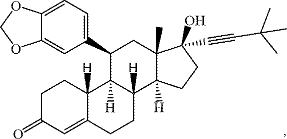

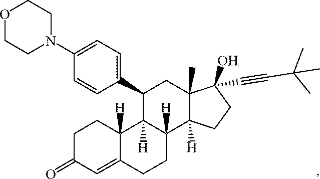

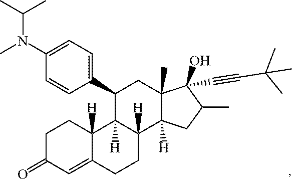

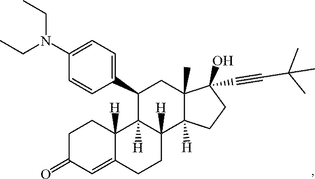

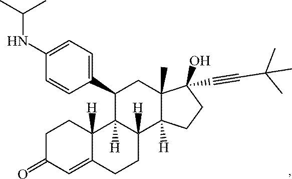

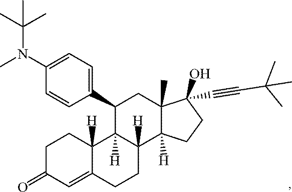

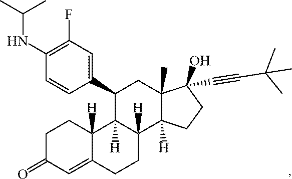

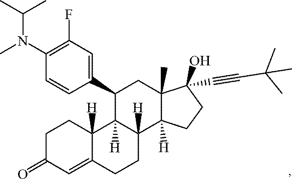

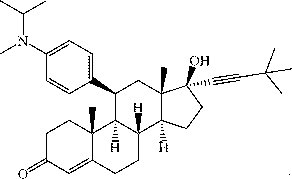

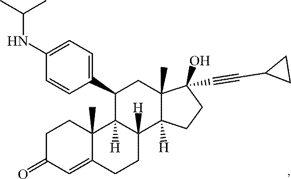

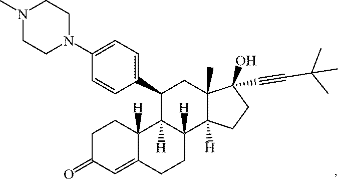

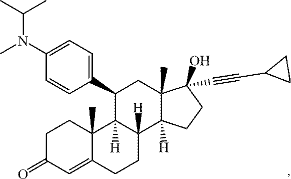

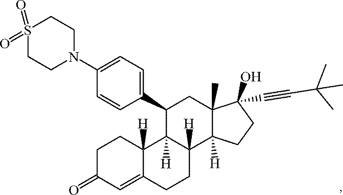

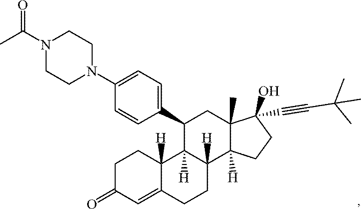

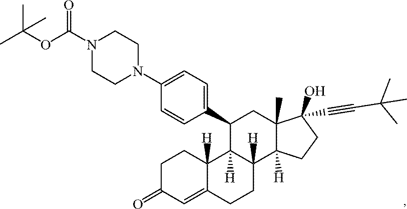

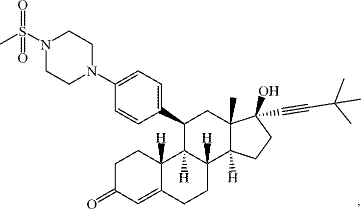

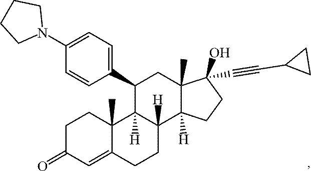

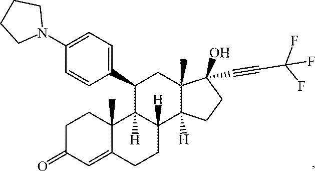

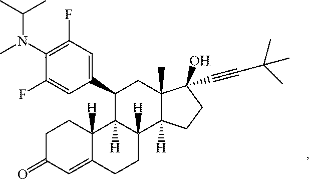

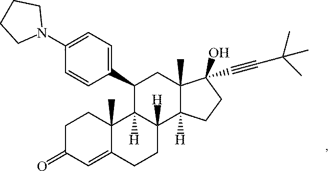

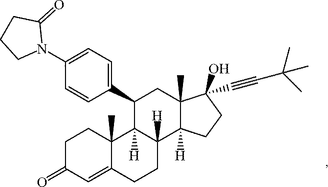

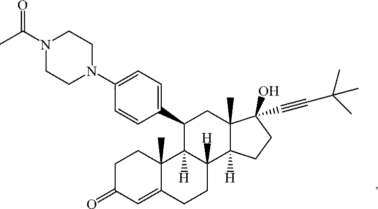

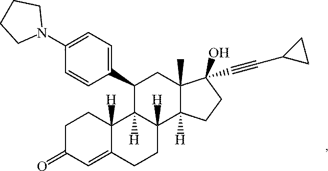

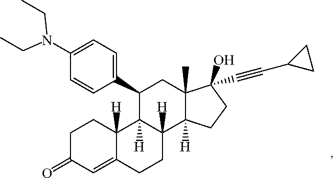

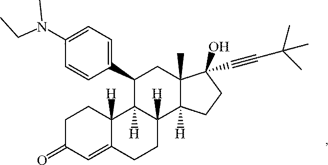

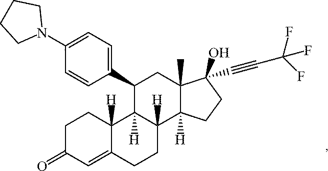

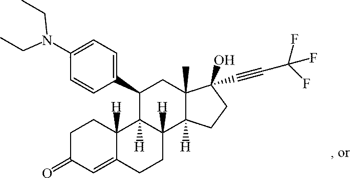

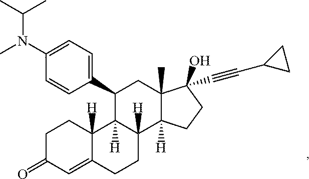

or a pharmaceutically acceptable salt thereof.

### Androgen Receptor (AR) Degraders

Disclosed herein are methods of treating prostate cancer, wherein the methods comprise administering a glucocorticoid receptor (GR) antagonist in combination with an androgen receptor (AR) degrader. In some embodiments, the AR degrader is ARV-110, ARV-330, SARD279, SARD033, ARCC-4, UT-34, ARD-111, ARD-86, ARD-77, ARD-69, ARD-61, LX-1, or LX-2. In some embodiments, the AR degrader is ARV-110.

In some embodiments, the AR degrader is

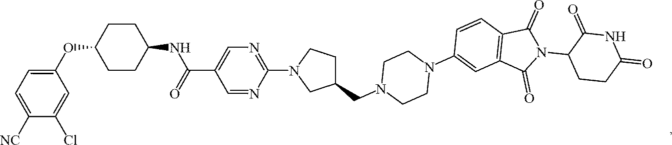

or pharmaceutically acceptable salt, solvate, or stereoisomer thereof.

In some embodiments, the AR degrader is

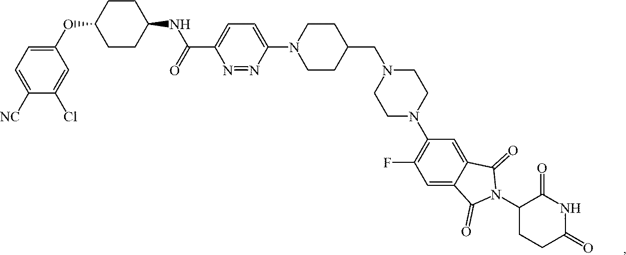

or pharmaceutically acceptable salt, solvate, or stereoisomer thereof.

In some embodiments, the AR degrader is

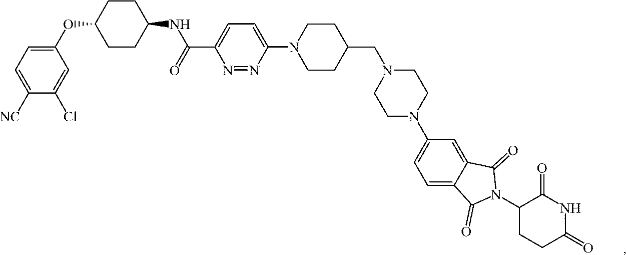

or pharmaceutically acceptable salt, solvate, or stereoisomer thereof.

In some embodiments, the AR degrader is

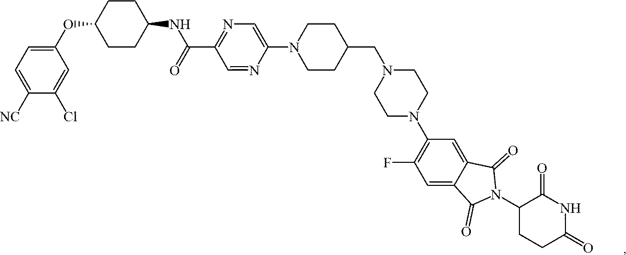

or pharmaceutically acceptable salt, solvate, or stereoisomer thereof.

In some embodiments, the AR degrader is

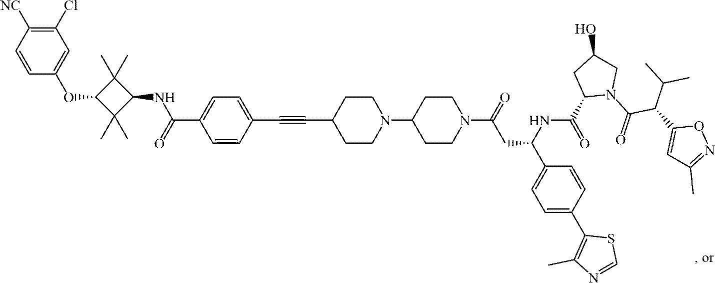

pharmaceutically acceptable salt, solvate, or stereoisomer thereof.

In some embodiments, the AR degrader is

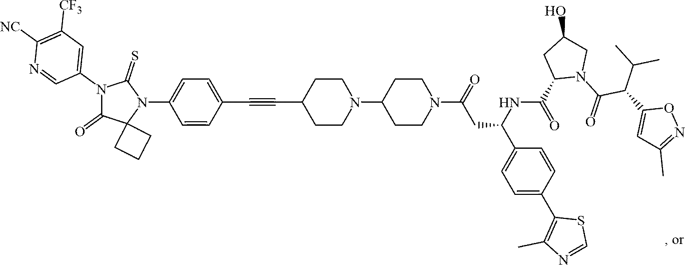

pharmaceutically acceptable salt, solvate, or stereoisomer thereof.

In some embodiments, the AR degrader is

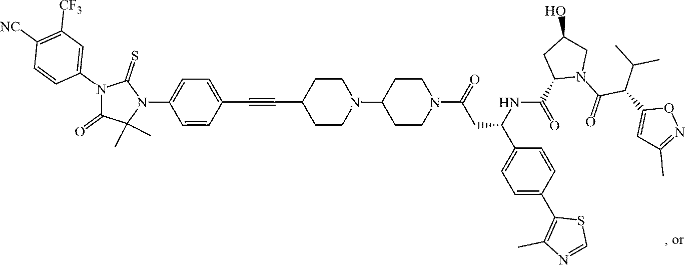

pharmaceutically acceptable salt, solvate, or stereoisomer thereof.

In some embodiments, the AR degrader is

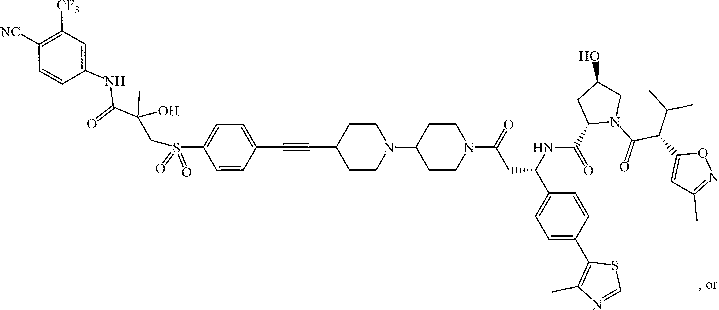

pharmaceutically acceptable salt, solvate, or stereoisomer thereof.

In some embodiments, the AR degrader is

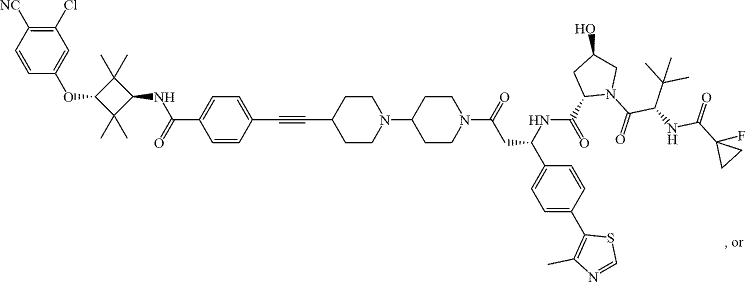

pharmaceutically acceptable salt, solvate, or stereoisomer thereof.

In some embodiments, the AR degrader is

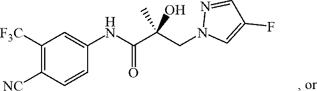

pharmaceutically acceptable salt, solvate, or stereoisomer thereof.

In some embodiments, the AR degrader is

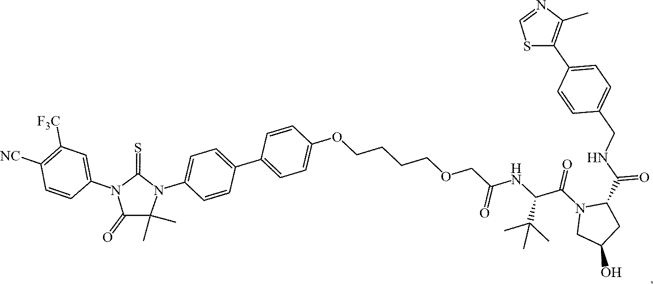

or pharmaceutically acceptable salt, solvate, or stereoisomer thereof.

In some embodiments, the AR degrader is

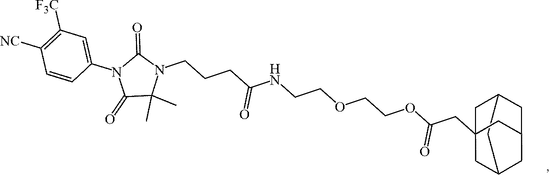

or pharmaceutically acceptable salt, solvate, or stereoisomer thereof.

In some embodiments, the AR degrader is

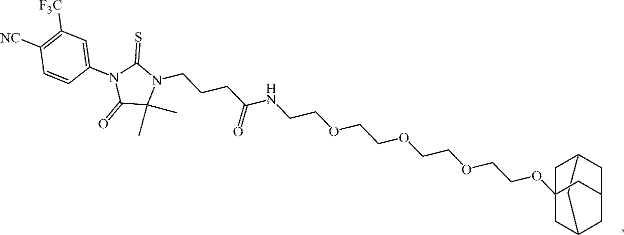

or pharmaceutically acceptable salt, solvate, or stereoisomer thereof.

In some embodiments, the AR degrader is

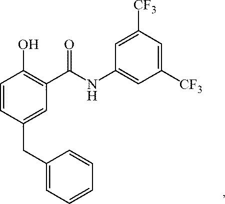

or pharmaceutically acceptable salt, solvate, or stereoisomer thereof.

In some embodiments, the AR degrader is f

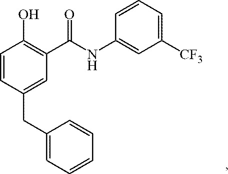

or pharmaceutically acceptable salt, solvate, or stereoisomer thereof.

In some embodiments, the AR degrader is

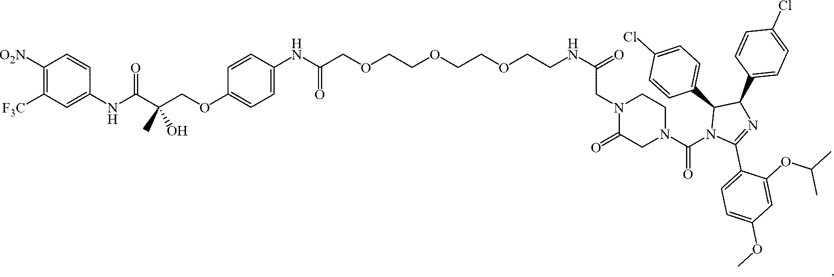

or pharmaceutically acceptable salt, solvate, or stereoisomer thereof.

In some embodiments, the AR degrader is ARV-110

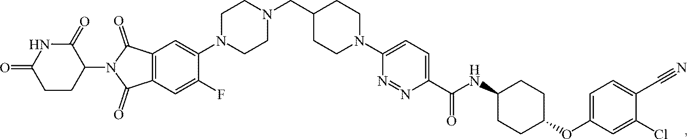

or a pharmaceutically acceptable salt, solvate, or stereoisomer thereof.

### Dosing and Administration

In one aspect, the compositions described herein are used for the treatment of prostate cancer. In addition, a method for treating prostate cancer in a subject in need of such treatment, involves administration of compositions in therapeutically effective amounts to said subject.

Suitable dosage forms include, for example, liquids, suspensions, powders for reconstitution, tablets, pills, sachets, or capsules of hard or soft gelatin (See, e.g., Remington: The Science and Practice of Pharmacy (Gennaro, 21^{st} Ed. Mack Pub. Co., Easton, PA (2005)).

Dosages of compositions described herein can be determined by any suitable method. Maximum tolerated doses (MTD) and maximum response doses (MRD) for the GR antagonist (e.g., compound 1, or a pharmaceutically acceptable salt thereof) and the AR degrader can be determined via established animal and human experimental protocols as well as in the examples described herein. For example, toxicity and therapeutic efficacy of the GR antagonist (e.g., compound 1, or a pharmaceutically acceptable salt thereof) and AR degrader can be determined by standard pharmaceutical procedures in cell cultures or experimental animals, including, but not limited to, for determining the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized. Additional relative dosages, represented as a percent of maximal response or of maximum tolerated dose, are readily obtained via the protocols.

In some embodiments, the GR antagonist (e.g., compound 1, or a pharmaceutically acceptable salt thereof) is administered in an amount between about 10 mg to 500 mg per day. In some embodiments, the AR degrader is administered in an amount between about 10 mg to 1000 mg per day.

In some embodiments, the amount of the GR antagonist (e.g., compound 1, or a pharmaceutically acceptable salt thereof) formulation that corresponds to such an amount varies depending upon factors such as the particular salt or form, disease condition and its severity, the identity (e.g., age, weight, sex) of the subject or host in need of treatment, but can nevertheless be determined according to the particular circumstances surrounding the case, including, e.g., the specific agent being administered, the liquid formulation type, the condition being treated, and the subject or host being treated.

In some embodiments, the amount of the AR degrader, or a pharmaceutically acceptable salt thereof formulation that corresponds to such an amount varies depending upon factors such as the particular salt or form, disease condition and its severity, the identity (e.g., age, weight, sex) of the subject or host in need of treatment, but can nevertheless be determined according to the particular circumstances surrounding the case, including, e.g., the specific agent being administered, the liquid formulation type, the condition being treated, and the subject or host being treated.

In prophylactic applications, the compositions described herein are administered to a patient susceptible to or otherwise at risk of prostate cancer. Such an amount is defined to be a “prophylactically effective amount or dose.” In this use, the precise amounts also depend on the patient’s age, state of health, weight, and the like. When used in a patient, effective amounts for this use will depend on the risk or susceptibility of developing the particular disease, previous therapy, the patient’s health status and response to the compositions, and the judgment of the treating physician.

In certain embodiments wherein the patient’s condition does not improve, upon the doctor’s discretion the administration of a composition described herein are administered chronically, that is, for an extended period of time, including throughout the duration of the patient’s life in order to ameliorate or otherwise control or limit the symptoms of the patient’s disease. In other embodiments, administration of a composition continues until complete or partial response of a disease.

In some embodiments, the GR antagonist (e.g., compound 1, or a pharmaceutically acceptable salt thereof) is administered once a day. In some embodiments, the GR antagonist (e.g., compound 1, or a pharmaceutically acceptable salt thereof) is administered twice a day. In some embodiments, the GR antagonist (e.g., compound 1, or a pharmaceutically acceptable salt thereof) is administered three times a day.

In some embodiments, the AR degrader is administered once a day. In some embodiments, the AR degrader is administered twice a day. In some embodiments, the AR degrader is administered three times a day.

In some embodiments, the GR antagonist (e.g., compound 1, or a pharmaceutically acceptable salt thereof) and/or the AR degrader are administered to a subject who is in a fasted state. A fasted state refers to a subject who has gone without food or fasted for a certain period of time. General fasting periods include at least 4 hours, at least 6 hours, at least 8 hours, at least 10 hours, at least 12 hours, at least 14 hours and at least 16 hours without food. In some embodiments, the GR antagonist (e.g., compound 1, or a pharmaceutically acceptable salt thereof) and/or the AR degrader are administered to a subject who is in a fasted state for at least 8 hours. In other embodiments, the GR antagonist (e.g., compound 1, or a pharmaceutically acceptable salt thereof) and/or the AR degrader are administered to a subject who is in a fasted state for at least 10 hours. In yet other embodiments, the GR antagonist (e.g., compound 1, or a pharmaceutically acceptable salt thereof) and/or the AR degrader are administered to a subject who is in a fasted state for at least 12 hours. In other embodiments, the GR antagonist (e.g., compound 1, or a pharmaceutically acceptable salt thereof) and/or the AR degrader are administered to a subject who has fasted overnight.

In other embodiments, the GR antagonist (e.g., compound 1, or a pharmaceutically acceptable salt thereof) and/or the AR degrader are administered to a subject who is in a fed state. A fed state refers to a subject who has taken food or has had a meal. In certain embodiments, a composition is administered to a subject in a fed state 5 minutes post-meal, 10 minutes post-meal, 15 minutes post-meal, 20 minutes post-meal, 30 minutes post-meal, 40 minutes post-meal, 50 minutes post-meal, 1 hour post-meal, or 2 hours post-meal. In certain instances, the GR antagonist (e.g., compound 1, or a pharmaceutically acceptable salt thereof) and/or the AR degrader are administered to a subject in a fed state 30 minutes post-meal. In other instances, the GR antagonist (e.g., compound 1, or a pharmaceutically acceptable salt thereof) and/or the AR degrader are administered to a subject in a fed state 1 hour post-meal. In yet further embodiments, the GR antagonist (e.g., compound 1, or a pharmaceutically acceptable salt thereof) is administered to a subject with food.

In some instances, the methods described herein further comprise administering the compositions and formulations comprising the GR antagonist (e.g., compound 1, or a pharmaceutically acceptable salt thereof) in combination with the AR degrader to the subject or patient in need thereof in multiple cycles repeated on a regular schedule with periods of rest in between each cycle. For example, in some instances, treatment given for one week followed by three weeks of rest is one treatment cycle.

The length of a treatment cycle depends on the treatment being given. In some embodiments, the length of a treatment cycle ranges from two to six weeks. In some embodiments, the length of a treatment cycle ranges from three to six weeks. In some embodiments, the length of a treatment cycle ranges from three to four weeks. In some embodiments, the length of a treatment cycle is three weeks (or 21 days). In some embodiments, the length of a treatment cycle is four weeks (28 days). In some embodiments, the length of a treatment cycle is 56 days. In some embodiments, a treatment cycle lasts one, two, three, or four weeks. In some embodiments, a treatment cycle lasts three weeks. In some embodiments, a treatment cycle lasts four weeks. The number of treatment doses scheduled within each cycle also varies depending on the drugs being given.

In some embodiments, the GR antagonist (e.g., compound 1, or a pharmaceutically acceptable salt thereof) and the AR degrader are administered to the subject in need thereof concurrently. In some embodiments, the GR antagonist (e.g., compound 1, or a pharmaceutically acceptable salt thereof) and the AR degrader are administered to the subject in need thereof consecutively.

### Kits and Articles of Manufacture

Disclosed herein, in certain embodiments, are kits and articles of manufacture for use with one or more methods and compositions described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for drugs, or the approved product insert. In one embodiment, compositions containing a compound provided herein formulated in a compatible pharmaceutical carrier are also prepared, placed in an appropriate container, and labeled for treatment of an indicated condition.

Disclosed herein is a kit comprising (i) a glucocorticoid receptor (GR) antagonist and (ii) an androgen receptor (AR) degrader and a package insert comprising instructions for using the (i) glucocorticoid receptor (GR) antagonist and (ii) androgen receptor (AR) degrader to treat prostate cancer in a subject in need thereof.

Disclosed herein is a kit comprising (i) a glucocorticoid receptor (GR) antagonist and (ii) an androgen receptor (AR) degrader for use in treating prostate cancer in a subject in need thereof and a package insert comprising instructions for measuring the expression of glucocorticoid receptor (GR) in one or more of the cells comprising the prostate cancer and using the (i) glucocorticoid receptor (GR) antagonist and (ii) androgen receptor (AR) degrader if one or more of the cells comprising the prostate cancer exhibits increased expression level of GR.

Disclosed herein is a kit comprising a glucocorticoid receptor (GR) antagonist for use in treating prostate cancer in a subject in need thereof and a package insert comprising instructions for measuring the expression of glucocorticoid receptor (GR) in one or more of the cells comprising the prostate cancer and using the glucocorticoid receptor (GR) antagonist if one or more of the cells comprising the prostate cancer exhibits increased expression level of GR. In some embodiments, the subject in need thereof was previously administered an androgen antagonist (AR) degrader for a period of time. In some embodiments, the period of time is between about 1 month and about 2 years.

### Definitions

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used herein, the term “ARV-110” means the compound having the structure below, Chemical Abstracts Registry No. 2222112-77-6, and having the chemical name N-[trans-4-(3-chloro-4-cyanophenoxy)cyclohexyl]-6-[4-[[4-[2-(2,6-dioxo-3-piperidinyl)-6-fluoro-2,3-dihydro-1,3-dioxo-1H-isoindol-5-yl]-1-piperazinyl]methyl]-1-piperidinyl]-3-pyridazinecarboxamide. The ARV-110 compound may be prepared by methods known to those having ordinary skill in the art, such as the methods described in U.S. Pat. Application Publication No. US 2018/0099940 A1, published Apr. 12, 2018, the contents of which are hereby incorporated by reference for that purpose

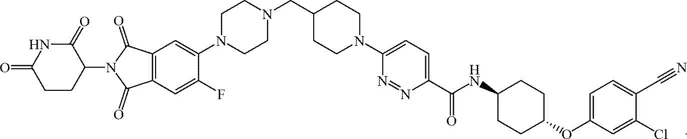

As used herein, the term “therapeutic” means an agent utilized to treat, combat, ameliorate, prevent or improve an unwanted condition or disease of a patient.

“Administering” when used in conjunction with a therapeutic means to administer a therapeutic systemically or locally, as directly into or onto a target tissue, or to administer a therapeutic to a patient whereby the therapeutic positively impacts the tissue to which it is targeted. Thus, as used herein, the term “administering”, when used in conjunction with a composition described herein, can include, but is not limited to, providing a composition into or onto the target tissue; providing a composition systemically to a patient by, e.g., oral administration whereby the therapeutic reaches the target tissue or cells. “Administering” a composition may be accomplished by injection, topical administration, and oral administration or by other methods alone or in combination with other known techniques.

The term “animal” as used herein includes, but is not limited to, humans and non-human vertebrates such as wild, domestic and farm animals. As used herein, the terms “patient,” “subject” and “individual” are intended to include living organisms in which certain conditions as described herein can occur. Examples include humans, monkeys, cows, sheep, goats, dogs, cats, mice, rats, and transgenic species thereof. In a preferred embodiment, the patient is a primate. In certain embodiments, the primate or subject is a human. In certain instances, the human is an adult. In certain instances, the human is child. In further instances, the human is under the age of 12 years. In certain instances, the human is elderly. In other instances, the human is 60 years of age or older. Other examples of subjects include experimental animals such as mice, rats, dogs, cats, goats, sheep, pigs, and cows. The experimental animal can be an animal model for a disorder, e.g., a transgenic mouse with hypertensive pathology.

By “pharmaceutically acceptable”, it is meant the carrier, diluent or excipient must be compatible with the other ingredients of the formulation and not deleterious to the recipient thereof.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology).

The terms “treat,” “treated,” “treatment,” or “treating” as used herein refers to both therapeutic treatment in some embodiments and prophylactic or preventative measures in other embodiments, wherein the object is to prevent or slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. A prophylactic benefit of treatment includes prevention of a condition, retarding the progress of a condition, stabilization of a condition, or decreasing the likelihood of occurrence of a condition. As used herein, “treat,” “treated,” “treatment,” or “treating” includes prophylaxis in some embodiments.

The term “resistant” as used herein refers to the cancer being no longer responsive to the treatment administered (e.g., AR degrader or AR inhibitor). A determination of whether a cancer, or one or more cells comprising a cancer, in a subject have become resistant to a specific treatment modality can be made by methods to known to those of ordinary skill in the art. For example, responsiveness, or non-responsiveness, as the case may be, of a cancer in a subject, or one or more cells comprising the cancer in a subject, can be assessed by measuring prostate-specific antigen (PSA) levels (by, for example, reference to Prostate Cancer Working Group 3 (PCWG3) criteria), increases or decreases in tumor size, use of Response Evaluation Criteria in Solid Tumors (RECIST response) (see, for example, Schwartz, et. al., Eur. J. Cancer, July 2016, vol. 62, pp. 132-137, for a description of RECIST v1.1), or progression-free survival.

“Alkyl” refers to a straight or branched chain hydrocarbon monoradical, which may be fully saturated or unsaturated, having from one to about ten carbon atoms, or from one to six carbon atoms. Examples of saturated hydrocarbon monoradical include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. When the alkyl refers to an unsaturated straight or branched chain hydrocarbon monoradical it is known as an “alkenyl” or an “alkynyl”. The alkenyl may be in either the cis or trans conformation about the double bond(s), and should be understood to include both isomers. Examples of alkenyls include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Examples of alkynyl include, but are not limited to ethynyl, 2-propynyl, 2-butynyl, 1,3-butadiynyl and the like. Whenever it appears herein, a numerical range such as “C₂-C₆ alkynyl” means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkyl group is optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN═, —CF₃, —OH, or —OMe. In some embodiments, the alkyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen.

“Alkoxy” refers to a radical of the formula -ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic or polycyclic carbocyclic ring, which may include fused (when fused with an aryl or a heteroaryl ring, the cycloalkyl is bonded through a non-aromatic ring atom) or bridged ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Heterocycloalkyl” refers to a 3- to 24-membered partially or fully saturated ring radical comprising 2 to 23 carbon atoms and from one to 8 heteroatoms selected from nitrogen, oxygen, phosphorous, and sulfur. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon or sulfur atoms in the heterocycloalkyl radical may be optionally oxidized; the nitrogen atom may be optionally quaternized. In some embodiments, the heterocycloalkyl is a 3- to 6-membered heterocycloalkyl. In some embodiments, the heterocycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroalkyl” refers to an alkyl group in which one or more skeletal atoms of the alkyl are selected from an atom other than carbon, e.g., oxygen, nitrogen (e.g. —NH—, -N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH, -N(alkyl)-), sulfur, or combinations thereof wherein the heteroalkyl is attached to the rest of the molecule at a carbon atom of the heteroalkyl. Unless stated otherwise specifically in the specification, a heteroalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heteroaryl” refers to a 5- to 14-membered ring system radical comprising hydrogen atoms, one to thirteen carbon atoms, one to six heteroatoms selected from nitrogen, oxygen, phosphorous, and sulfur, and at least one aromatic ring. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

### EXAMPLES

### Methods and Reagents for Examples 1 to 5

### Cell Culture

LNCaP (ATCC), CWR22PC (procured from Dr. Charles Sawyer’s lab, MSKCC), 22Rv1 (ATCC), LAPC4 (ATCC), LREX’ (procured from Dr. Charles Sawyer’s lab, MSKCC), H660 (ATCC), PC3 (ATCC), and DU145 (ATCC) cells were maintained in RPMI-1640 media (Corning #14-040-CM), and VCaP (ATCC) cells was maintained in DMEM media (Corning #15-017-CM) supplemented with 10% FBS (OmegaScientific #FB11) containing high testosterone, 1% Penicillin-Streptomycin (Corning #30-002-CI), 1% L-glutamine (Corning #25-005-CI), and 1% Antibiotic-Antimycotic (Corning #30-004-CI).

### PROTAC® AR Degrader Treatment Experiments

### Dose-Dependent AR Degrader Treatment

LNCaP and CWR22PC cells were plated in seven 15-cm dishes (Corning#353025) at a density of 5 million cells per dish in RPMI-1640 media supplemented with 10% charcoal-stripped serum (CSS; OmegaScientific#FB4). After 24 hours, the cells were treated with 0, 3, 10, 30, 100, 300 and 1000 nM of ARD1 or ARD2 and incubated at 37° C. for 3 days. The cells were then trypsinized (Corning#25-053-CI) and harvested for transcript (RT-qPCR) and protein (western blot) analysis.

### Time-Dependent AR Degrader Treatment

5 million of LNCaP and CWR22PC cells per dish were plated in five 15-cm dishes in 20 mL of RPMI-1640 media supplemented with 10% charcoal-stripped serum (CSS) and cultured at 37° C. for 24 hours. Cells were treated with 30 nM of dexamethasone and with 100 nM of ARD1 or ARD2 and maintained at 37° C. until harvest. On Day 0, 6, 10, 13, and 15, cells were trypsinized, cell pellets were washed with 1XPBS and then divided for mRNA and protein preparation.

### AR Degraders in Combination With Compound 1 in Prostate Cancer Cells

CWR22PC cells were seeded at a density of 2 × 10⁵ cells/well in 6-well plates (Corning# 3516) in 2 mL of RPMI-1640 media supplemented with 10% charcoal-stripped serum (CSS) in duplicate. 24 hours later, cells were treated with 100 pM R1881, 30 nM dexamethasone, 0.5 µM compound 1, 1 µM of ARD1 or ARD2, or combinations thereof as indicated. Fresh media containing the indicated treatment was replaced every 5-7 days. Cell supernatant was collected on Day 12 and 19 and stored at -80° C. for PSA AlphaLISA assay. Cell images were taken on Day 12 and 19 using Leica DMi8 microscope (LEICA MICROSYSTEMS, INC. IL) with a 10X magnification. Cells were then trypsinized and cell numbers in each treatment group were counted three times, average counts were plotted with GraphPad Prism 8. Cell pellets were washed once with 1XPBS and then lysed in 600 µL RLT RNA lysis buffer and stored at -80° C. RNA was extracted for end of study (EOS) biomarker analysis for which the detailed procedures are described in the section RNA Extraction and RT-qPCR analysis.

### Biomarker Analysis by RT-qPCR

CWR22PC and 22RV1 cells were seeded at a density of 2 × 10⁵ cells/well in 6-well plates in 2 mL of RPMI-1640 media supplemented with 10% charcoal-stripped serum (CSS). Cells were treated with 100 pM R1881, 30 nM dexamethasone, 0.5 µM compound 1, 1 µM of ARD1 or ARD2, or combinations thereof as indicated and maintained at 37° C. 48 hours later, media was removed, and cells were washed once with PBS and then harvested for mRNA analysis.

### Target Gene Assay

10,000 LNCaP and CWR22PC cells per well were plated in triplicate in poly-lysine coated 96-well plates (ThermoFisher#152039) in 200 µL of RPMI-1640 media supplemented with 10% charcoal-stripped serum. 24 hours later, cells were treated with 100 pM of R1881 and 8-point dilution for enzalutamide, ARD1 or ARD2 with concentrations ranging from 1 µM to 0.46 nM. After incubation at 37° C. for 24 hours, media was removed, and cells were washed once with PBS. 55 µL per well of iScript RT-qPCR sample preparation reagent (Bio-Rad #1708898) was added and incubated for 2 minutes before lysate was collected for mRNA analysis. RT-qPCR was set up in triplicate using 3 µL of lysate plus 7 µL of pre-mixed SensiFAST™ SYBR No-ROX One-Step Kit reagents (BIOLINE, Cat# BIO-98005) per reaction. The RT-qPCR reactions were run on a Bio-Rad CFX384 Real-Time PCR System and data was plotted with GraphPad Prism 8 software.

### RNA Extraction and RT-qPCR Analysis

Adherent cells or cell pellets harvested for mRNA analysis were first washed once with PBS and then resuspended in 600 µL of RLT lysis buffer (Qiagen Inc.) supplemented with 1% of betamercaptoethanol. RNA was extracted by QIAcube using the RNeasy Mini QIAcube kit (Qiagen#74116) according to manufacturer’s instructions. The extracted RNA samples were then quantified using a Nanodrop 8000 Spectrophotometer. RT-qPCR reactions were set up in triplicate using 3 µL of 3 ng/ µL of RNA in 7 µL of pre-mixed SensiFAST™ SYBR No-ROX One-Step Kit reagents (BIOLINE# BIO-98005). The RT-qPCR reactions were run on a Bio-Rad CFX384 Real-Time PCR System and data was plotted with GraphPad Prism 8 software.

Primer sequences for RT-qPCR

| Species | Genes | SEQ ID NO. | Forward Primers |
|---|---|---|---|
| Human | RPL27 | 1 | CTGGAATTGACCGCTACCC |
| | AR | 2 | CTTACACGTGGACGACCAGA |
| | GR | 3 | GTGAAATGGGCAAAGGCAATAC |
| | KLK3 | 4 | TGAGAGAGTGGAGAGTGACAT |
| | KLK4 | 5 | GGAACTCTTGCCTCGTTTCT |
| | NKX3.1 | 6 | GAGAGAGCCTTGGCCATATTC |
| | FKBP5 | 7 | GTACACAGCCAGTAGCCTAAAT |
| | GILZ | 8 | GGCAAAGGAGAAGGGTAGTT |
| | PER1 | 9 | ATCCAGTCCAGCCTTACCTA |
| | KLF9 | 10 | CACCTGACCCATACTCGTTTC |
| | SGK1 | 11 | CAGCACAACATCCACCTT CT |

| | | | |
|---|---|---|---|
| Species | Genes | SEQ ID NO. | Reverse Primers |
| Human | RPL27 | 12 | TGGGCATTAGGTGATTGTAGTT |
| | AR | 13 | GCTGTACATCCGGGACTTGT |
| | GR | 14 | CCCAGAGCAAATGCCATAAGA |
| | KLK3 | 15 | TCCAGGACAGAGTGGGTTAT |
| | KLK4 | 16 | GTCATAGAGCTTACTGCAGACC |
| | NKX3.1 | 17 | GAGAGAGCCTTGGCCATATTC |
| | FKBP5 | 18 | GAGAGCCATGCTCAATCTGT |
| | GILZ | 19 | ACAGTCGTTGTCAGGTGAAG |
| | PER1 | 20 | TTGGGACATAGGAGAAGAAAGC |
| | KLF9 | 21 | CTGTGCTAGTGATGGCTGTT |
| | SGK1 | 22 | CAGGCCATACAGCATCTCATAC |

### Western Blot Analysis

Cell pellets were washed with PBS and lysed in RIPA buffer (VWR#AAJ63306-AK) supplemented with proteasome inhibitor cocktails (VWR #AAJ65789-LQ). Resuspended cells were briefly sonicated for ~30 seconds using a probe sonicator and incubated on ice for 40 minutes, followed with centrifugation at 12,000 rpm for 15 minutes at 4° C. to collect the supernatant. Protein concentration was quantitated using Quick Start™ Bradford Protein Assay Kit (Bio-Rad #5000201).

50 µg/lane of protein was separated on NuPAGE™ 4-12% Bis-Tris Protein Gels (ThermoFisher Scientific#NP0335BOX). Proteins were transferred to nitrocellulose membranes using iBlot 2 Dry Blotting System (ThermoFisher#IB210021) at 20 V for 10 minutes. Membrane was first blocked using Intercept Blocking Buffer (LI-COR Biosciences#927-60001) for 1 hour at room temperature, then probed with primary antibodies diluted in blocking buffer supplemented with 1% of Tween-20 (BIO-RAD#1610781) and incubated overnight at 4° C. Next, membranes were washed 3 × 5 minutes with 1X TBS (Bio-Rad #1706435) supplemented with 1% Tween 20 (TBST) on shaker, then probed with secondary antibody at room temperature for 1 hour. Last, membrane was washed 3 × 10 minutes with TBST and one time with TBS for 10 minutes, and then developed using Odyssey CLx Imaging System (LI-COR Biosciences#9140). Primary antibodies: AR (CST#5153S), GR (CST#12041), Actin (CST#8457).

### PSA AlphaLISA Assay

Cell supernatant was collected from the experiment groups for various treatment conditions and time. PSA levels were determined using PSA (human) AlphaLISA Detection Kit (PerkinElmer #AL228C) according to manufacturer’s instructions. Specifically, 5 µL of sample was added to 20 µL with 10 µg/mL of AlphaLISA Anti-Analyte Acceptor beads and 1 nM of Biotinylated Antibody Anti-Analyte in a 96-well plate (Greiner#781904) and incubated for 1 hour at room temperature. 25 µL with 40 µg/mL of 2X Streptavidin Donor beads was then added and incubated for 30 minutes in the dark at room temperature. The plate was then read on Tecan Spark Plate Reader (Tecan Inc. NC). A standard curve was generated using the human PSA analyte provided in the kit by plotting AlphaLISA counts vs concentration of PSA (pg/mL). The standard curve was used to determine unknown PSA concentration from the experiment samples. The measured PSA concentrations were converted into concentration of ng per mL (ng/mL) and data was plotted in GraphPad Prism 8.

### Example 1. Prostate Cancer Cell Line Characterization and Selection

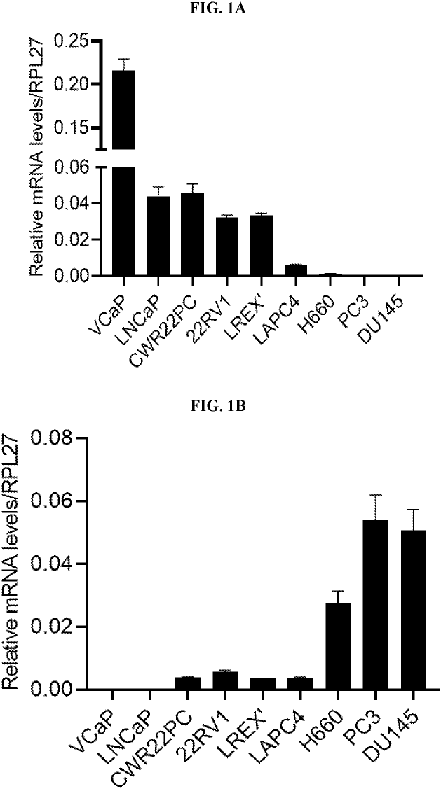

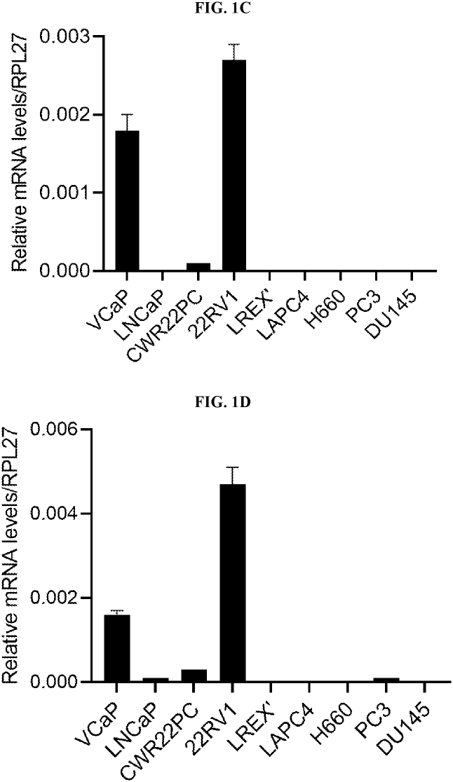

Whether GR is a resistance mechanism to PROTAC® AR degraders in prostate cancer and whether this resistance can be overcome by a GR antagonist was investigated. To that end, in vitro prostate cancer models to enable model selection were characterized. RT-qPCR was used to measure the expression levels of AR, GR, AR-V1 and AR-V7 in prostate cancer (PC) cell lines including VCaP, LNCaP, CWR22PC, 22Rv1, LREX’, LAPC4, H660, PC3 and DU145 (FIG. 1A, FIG. 1B, FIG. 1C, and FIG. 1D). Among these lines, VCaP, LNCaP and CWR22PC have established in vitro AR/androgen sensitivity and express AR mRNA to various extents. VCaP additionally expresses the AR variants AR-V1 and AR-V7 that are not degraded by ligand binding domain (LBD) binding PROTAC® AR degraders (FIG. 1C and FIG. 1D). LNCaP and CWR22PC were chosen as models for examining GR levels after chronic AR degrader treatment. These two cell lines further differ in their GR mRNA level, with LNCaP cells lacking GR expression and CWR22PC cells moderately expressing GR (FIG. 1B), which enable the assessment of the impact of chronic AR degrader treatment on GR levels in function of baseline GR expression. For the in vitro efficacy studies, CWR22PC cells were used to examine whether GR is a resistance mechanism to AR degraders and whether a GR antagonist such as compound 1 reverses this resistance. The in vitro AR sensitivity of CWR22PC cells enables the quantification of cell growth inhibition by AR degrader treatment, while the moderate baseline GR expression additionally allows the quantification of potential GR-driven cell growth in response to glucocorticoids. Biomarker studies were performed to evaluate GR and/or AR target genes under different treatment conditions and examine whether GR may replace AR and transcriptionally regulate a portion of AR target genes. AR sensitive line CWR22PC and AR-insensitive line 22Rv1 were used, with these cell lines showing comparable mRNA levels for both AR and GR (FIG. 1A and FIG. 1B).

### Example 2. PROTAC® AR Degraders Eliminate AR Protein, Inhibit AR Target Gene Expression, and Blunt Androgen Promoted Cell Growth in Prostate Cancer Cells

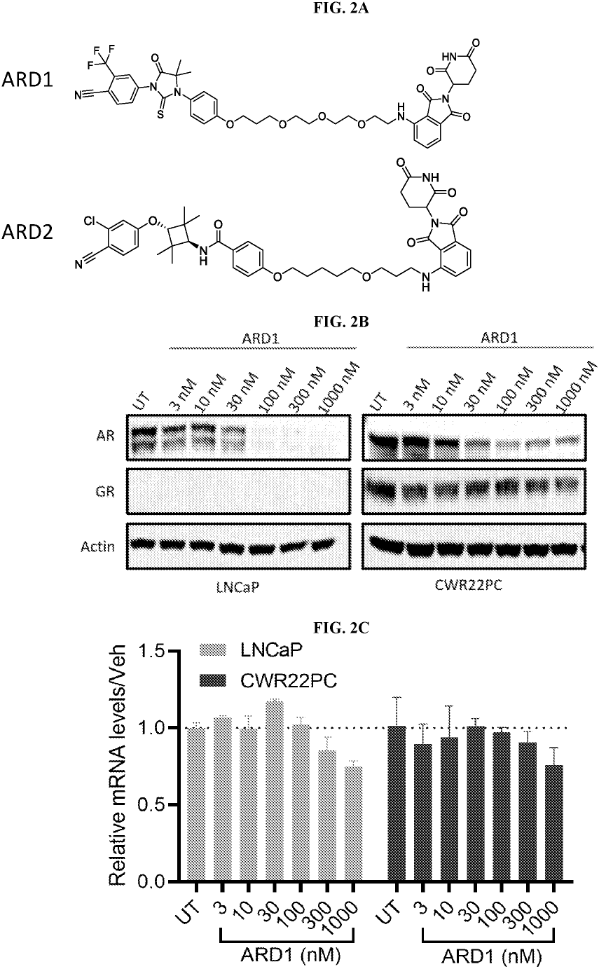

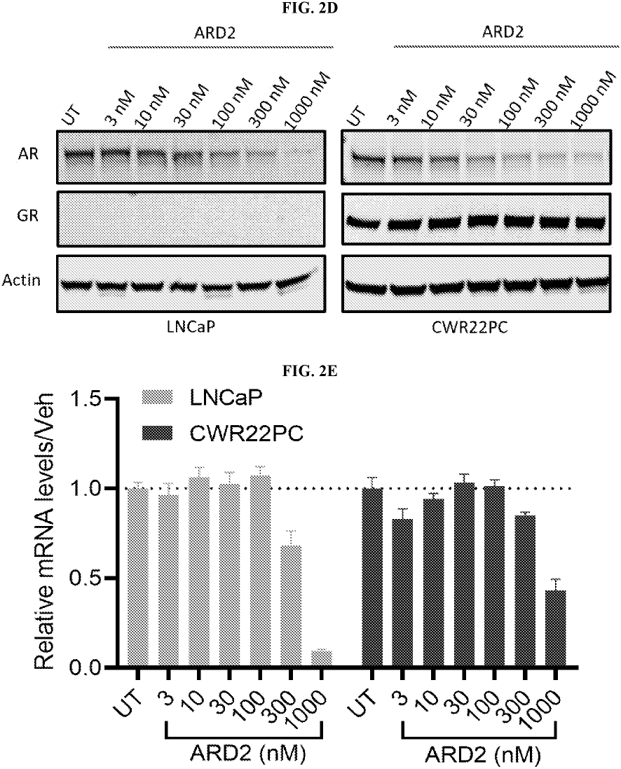

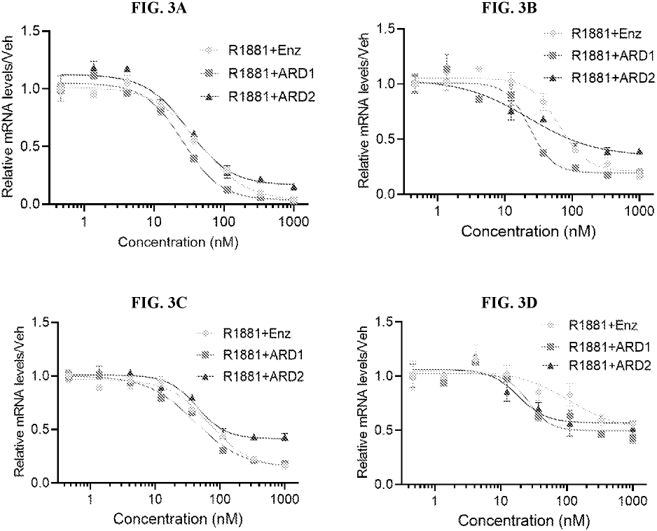

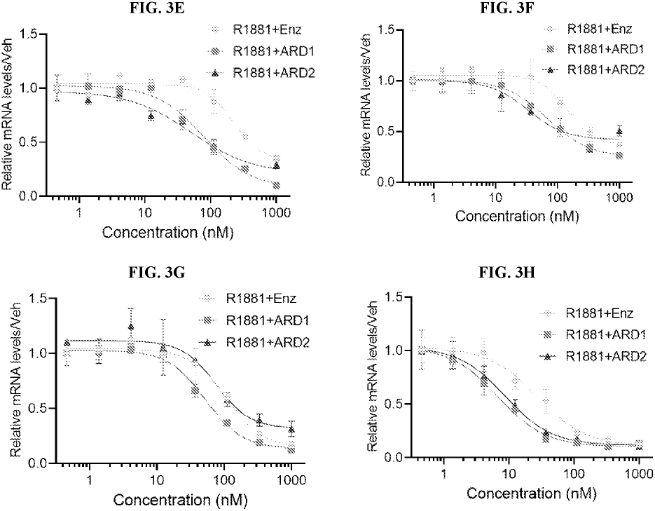

Two PROTAC® AR degraders, ARD1 and ARD2 (FIG. 2A), corresponding to examples 7 and 13 from US 20180099940, were synthesized. To verify the ability of these compounds to degrade AR protein, LNCaP and CWR22PC prostate cancer cells were treated with vehicle (untreated, UT) or increasing doses of ARD1 or ARD2 for three days. Treated cells were harvested to measure AR and GR protein by western blot analysis and mRNA levels by RT-qPCR analysis. The results showed that ARD1 degrades AR protein in a dose-dependent manner (FIG. 2B), with minimal impact on GR protein (FIG. 2B) and AR transcript levels (FIG. 2C). Similar results were obtained for ARD2 (FIG. 2D and FIG. 2E). These results suggest that ARD1 and ARD2 specifically reduce AR protein levels through protein degradation. To further solidify compounds ARD1 and ARD2 as AR degraders, the impact of both compounds on AR target gene expression using target gene assays was measured. In both LNCaP and CWR22PC cells, ARD1 and ARD2 inhibit the expression of AR target genes including KLK3, KLK4, NKX3.1, and FKBP5 in a dose-dependent manner with a similar or greater potency than AR antagonist enzalutamide (Enz) (FIGS. 3A-3H), suggesting that ARD1 and ARD2 potently block AR signaling in preclinical prostate cancer models.

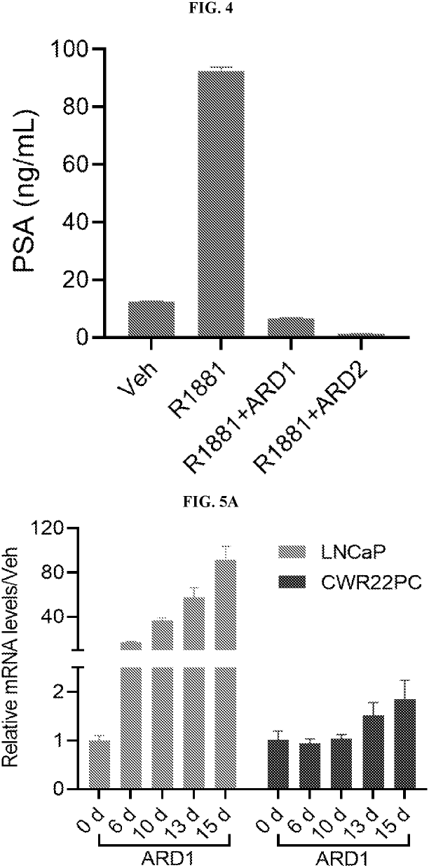

The impact of ARD1 and ARD2 on the growth of prostate cancer cells was explored. CWR22PC cells were plated in charcoal-stripped serum (CSS) media and treated with vehicle or 0.1 nM R1881 (synthetic AR ligand) without or with 1 µM of ARD1 or ARD2. Media with treatments were replaced every 5-7 days and cells were maintained at 37° C. for 22 days. Supernatant of each group was harvested at day 22 to measure PSA secretion by PSA AlphaLISA assays. The results showed that the addition of R1881 promotes CWR22PC cell growth and PSA secretion (FIG. 4), while ARD1 and ARD2 completely block R1881-induced effects.

Taken together, these results confirm that ARD1 and ARD2 are AR degraders that degrade AR protein, block AR signaling, and blunt androgen-mediated cell growth in prostate cancer models.

### Example 3. GR mRNA and Protein Levels Are Upregulated in Response to AR Degrader Treatment in CRPC Cells in a Time Dependent Manner

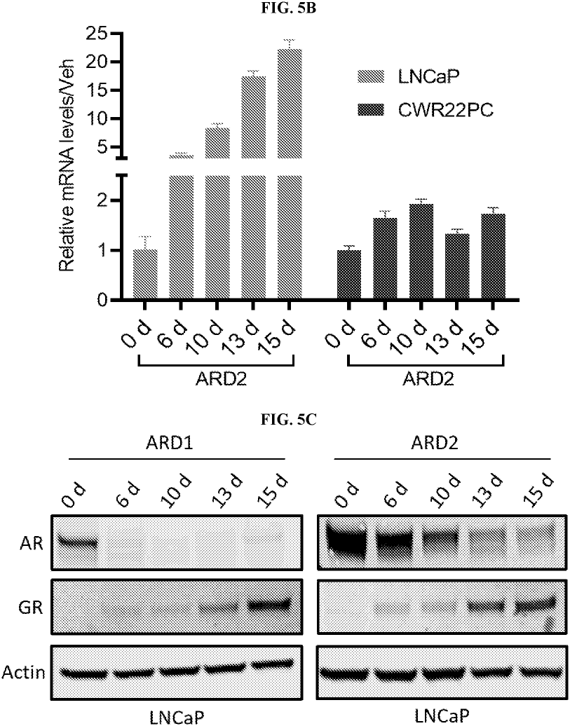

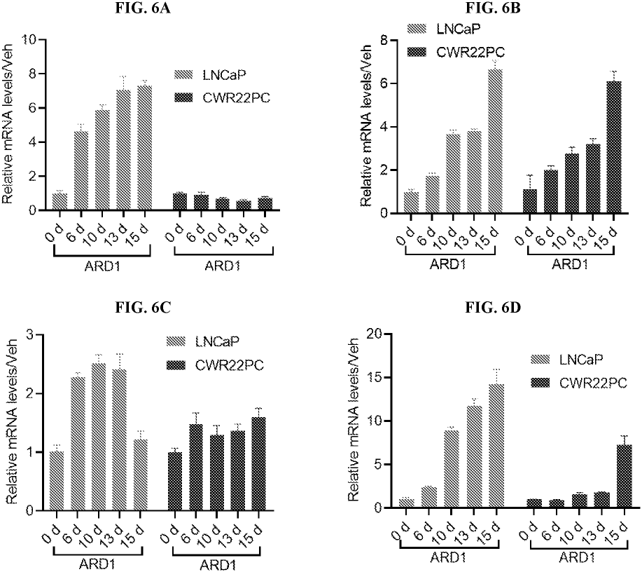

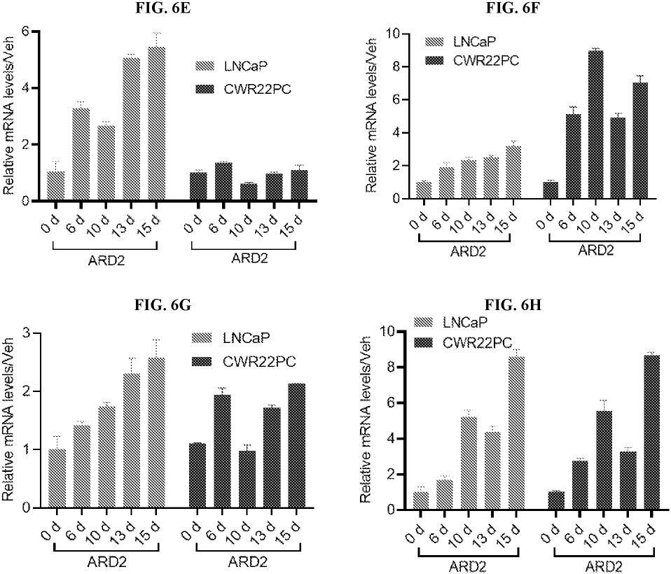

The potential impact of chronic AR degrader treatment on GR levels was explored. Given that cortisol levels in the culture media are insufficient to activate GR signaling, LNCaP and CWR22PC cells were seeded at 5 million cells per 15 cm dish at 20 mL CSS media supplemented with 30 nM of GR agonist dexamethasone. Cells were treated with 100 nM of ARD1 or ARD2 for 0, 6, 10, 13, and 15 days, and harvested for RT-qPCR and western blot analysis simultaneously. The results showed that ARD1 and ARD2 drastically upregulate GR mRNA and protein levels in GR-negative LNCaP cells (FIG. 5A, FIG. 5B, and FIG. 5C). In CWR22PC cells that already moderately express GR pre-treatment, AR degrader treatment modestly induced GR transcript levels (FIG. 5A and FIG. 5B), with minimal impact on GR protein levels (data not shown). The modest GR upregulation post AR degrader treatment in CWR22PC cells may be caused by higher baseline GR levels (FIG. 1B) and/or incomplete AR degradation (FIG. 2B and FIG. 2D). Concurrently, the expression of GR target genes including GILZ, PER1, KLF9, and SGK1 in LNCaP and CWR22PC cells post AR degrader treatment were measured. The results showed that most of the GR target genes are upregulated in a time dependent manner in both cell lines with both compounds (FIG. 6A - FIG. 6H), suggesting that chronic AR degrader treatment not only upregulates GR but also activates or enhances GR signaling.

Together, these results indicate that AR degrader treatment upregulates GR transcript, GR protein and GR signaling in prostate cancer cells.

### Example 4. Compound 1 Reverses Glucocorticoid-Induced AR and/or GR Target Genes in Response to AR Degrader Treatment

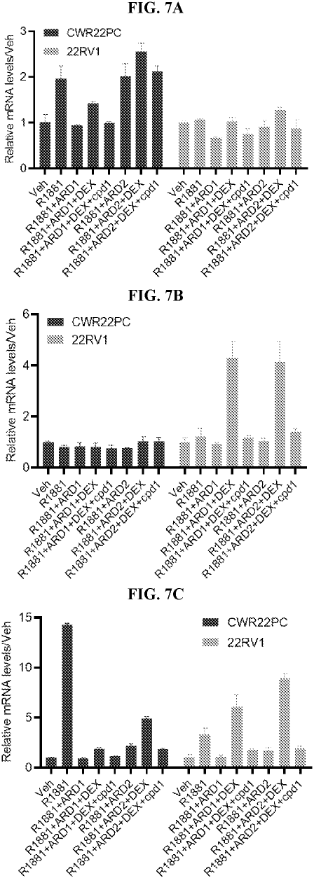

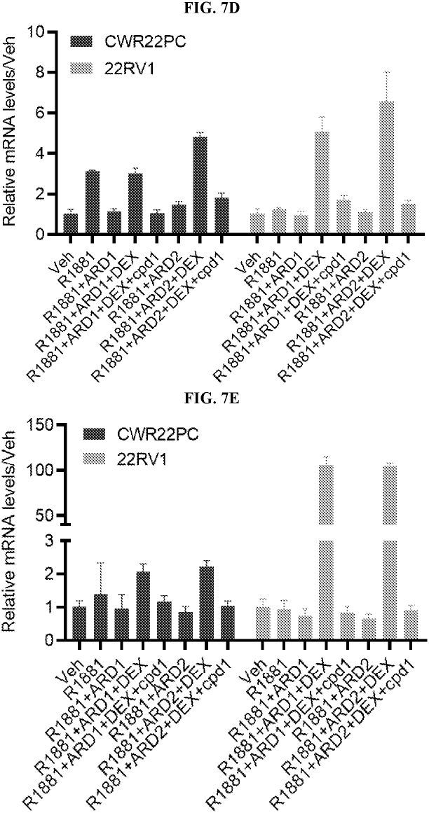

Whether GR rescues AR signaling blocked by AR degraders and whether AR degraders in combination with GR antagonist compound 1 achieve complete signaling inhibition in response to both androgen and glucocorticoid stimulation was investigated. GR-moderate CWR22PC and 22Rv1 prostate cancer cells were treated with vehicle, 0.1 nM of R1881, 1 µM of ARD1 or ARD2, 30 nM of dexamethasone (DEX), and 0.5 µM of compound 1 or combinations thereof (FIG. 7A - FIG. 7E). 48 hours post treatment, RNA was extracted, and RT-qPCR analysis was performed to measure the transcript levels of AR and/or GR target genes including KLK4, KLF9, FKBP5, PER1, and SGK1. The results showed that the expression induction by R1881 for AR targets KLK4, FKBP5 and PER1 is completely inhibited by either ARD1 or ARD2 treatment, except for KLK4 expression upon ARD2 treatment. Furthermore, activation of GR with synthetic glucocorticoid dexamethasone (DEX) rescues a portion of AR targets such as KLK4, FKBP5 and PER1, and induces GR targets such as KLF9 and SGK1. Treatment with GR antagonist Compound 1 fully reverses DEX-induced GR and AR target gene expression in the presence of either AR degrader. The data suggest that in GR-positive prostate models, beyond activating GR signaling, glucocorticoids also induce a subset of AR target genes upon AR protein loss from AR degrader treatment, and these effects are fully reversed with compound 1 co-treatment.

### Example 5. Compound 1 Overcomes Glucocorticoid-Mediated Resistance to AR Degrader Treatment

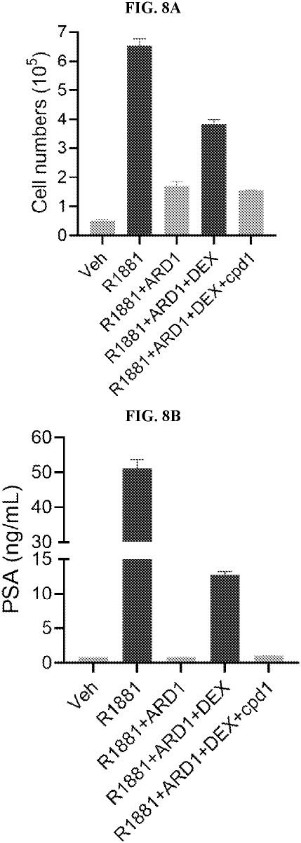

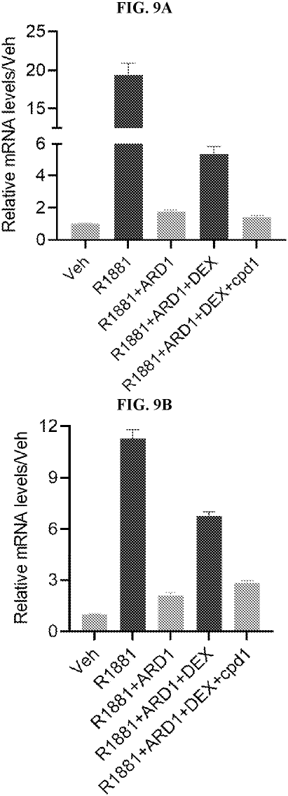

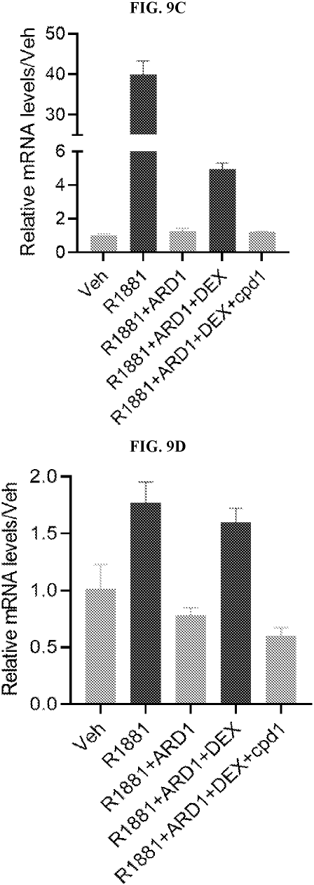

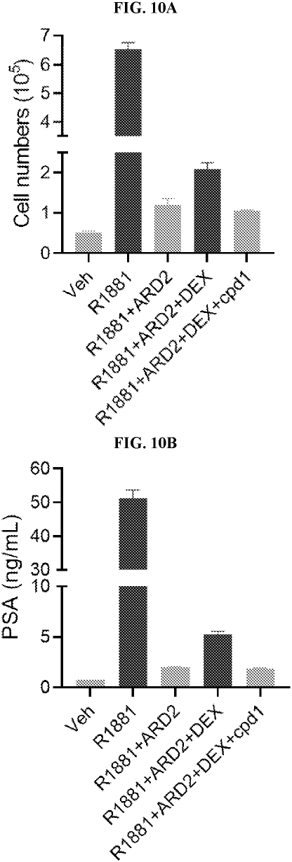

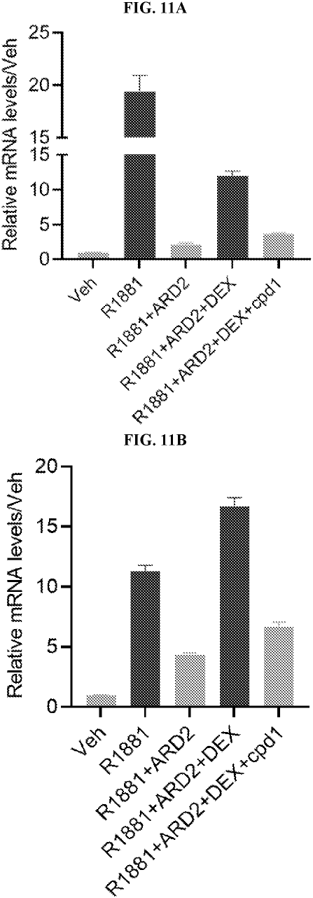

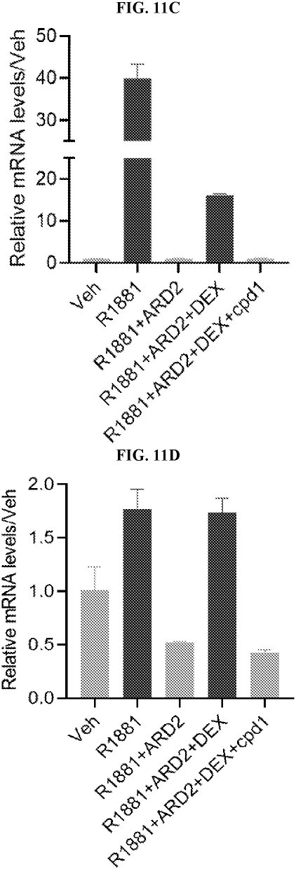

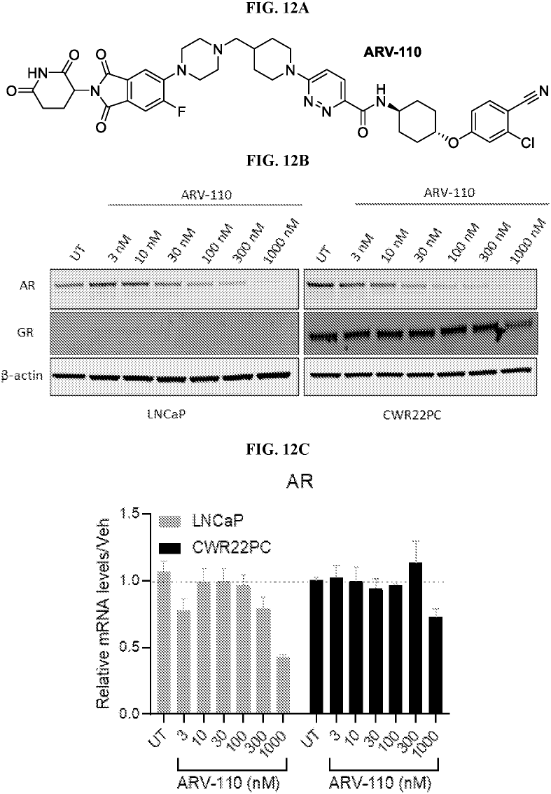

The functional consequences of GR upregulation on prostate cancer cells was investigated. CWR22PC cells were seeded in CSS media and treated with vehicle, 0.1 nM of R1881, 1 µM of ARD1 or ARD2, 30 nM of dexamethasone, and 0.5 µM of compound 1 or combinations thereof as indicated in FIG. 8A, FIG. 8B, FIG. 10A, and FIG. 10B. Fresh media with treatments was replaced every 5-7 days and cells were maintained at 37° C. for 19 days before final harvest. Cell supernatants were harvested for secreted PSA measurement using the PSA AlphaLISA assay (FIG. 8B and FIG. 10B). On day 19 at end of study (EOS), cells were trypsinized and resuspended for cell number counting, while the remaining cells were pelleted and used for RNA extraction and EOS biomarker analysis using RT-qPCR target gene assays. The results showed that ARD1 and ARD2 treatment blunts R1881-induced cell growth (FIG. 8A and FIG. 10A), PSA secretion (FIG. 8B and FIG. 10B), and target gene expression (FIG. 9A - FIG. 9D and FIG. 11A - FIG. 11D). 30 nM of dexamethasone partially rescues ARD1 and ARD2 effects by restoring cell growth, PSA secretion, and biomarker expression. Importantly, the addition of compound 1 fully reverses these effects. These results indicate that GR may be a resistance mechanism to AR degraders, and that compound 1 in combination with AR degraders achieves better efficacy when GR signaling is active in preclinical prostate cancer models.

### Methods and Reagents for Examples 6 to 9:

### Cell Culture

LNCaP (ATCC), CWR22PC (procured from Dr. Charles Sawyer’s lab, MSKCC), and 22Rv1 (ATCC) were maintained in RPMI-1640 media (Corning #14-040-CM) supplemented with 10% FBS (OmegaScientific #FB11) containing high testosterone, 1% Penicillin-Streptomycin (Corning #30-002-CI), 1% L-glutamine (Corning #25-005-CI), and 1% Antibiotic-Antimycotic (Corning #30-004-CI).

### Dose-Dependent AR Degrader Treatment

LNCaP and CWR22PC cells were plated in seven 10-cm dishes (Corning#353003) at a density of 2 million cells per dish in RPMI-1640 media supplemented with 10% charcoal-stripped serum (CSS; OmegaScientific#FB4). After 24 hours, the cells were treated with 0, 3, 10, 30, 100, 300 and 1000 nM of ARV-110 and incubated at 37° C. for 3 days. The cells were then trypsinized (Corning#25-053-CI) and harvested for transcript (RT-qPCR) and protein (western blot) analysis.

### Time-Dependent AR Degrader Treatment

2 million of LNCaP and CWR22PC cells per dish were plated in five 10-cm dishes in 10 mL of RPMI-1640 media supplemented with 10% charcoal-stripped serum (CSS) and cultured at 37° C. for 24 hours. Cells were treated with 30 nM of dexamethasone and with 100 nM of ARV-110 and maintained at 37° C. until harvest. On Day 0, 6, 10, 13, and 15, cells were trypsinized, cell pellets were washed with 1XPBS and then divided for mRNA and protein preparation.

### AR Degraders in Combination With Compound 1 in Prostate Cancer Cells

CWR22PC cells were seeded at a density of 2 × 10⁵ cells/well in 6-well plates (Corning# 3516) in 2 mL of RPMI-1640 media supplemented with 10% charcoal-stripped serum (CSS) in duplicate. 24 hours later, cells were treated with 100 pM R1881, 30 nM dexamethasone, 0.5 µM compound 1, 1 µM of ARV-110, or combinations thereof as indicated. Fresh media containing the indicated treatment was replaced every 5-7 days. Cell supernatant was collected on Day 15 and 21 and stored at -80° C. for PSA AlphaLISA assay. Cells were then trypsinized and cell numbers in each treatment group were counted three times, average counts were plotted with GraphPad Prism 8. Cell pellets were washed once with 1XPBS and then lysed in 600 µL RLT RNA lysis buffer and stored at -80° C. RNA was extracted for end of study (EOS) biomarker analysis for which the detailed procedures are described in the section RNA Extraction and RT-qPCR analysis.

### Biomarker Analysis by RT-qPCR

CWR22PC and 22Rv1 cells were seeded at a density of 2 × 10⁵ cells/well in 6-well plates in 2 mL of RPMI-1640 media supplemented with 10% charcoal-stripped serum (CSS). Cells were treated with 100 pM R1881, 30 nM dexamethasone, 0.5 µM compound 1, 1 µM of ARV-110, or combinations thereof as indicated and maintained at 37° C. 48 hours later, media was removed, and cells were washed once with PBS and then harvested for mRNA analysis.

### Target Gene Assay

15,000 LNCaP and CWR22PC cells per well were plated in triplicate in poly-lysine coated 96-well plates (ThermoFisher#152039) in 200 µL of RPMI-1640 media supplemented with 10% charcoal-stripped serum. 24 hours later, cells were treated with 100 pM of R1881 and 8-point dilution for enzalutamide or ARV-110 with concentrations ranging from 1 µM to 0.46 nM. After incubation at 37° C. for 24 hours, media was removed, and cells were washed once with PBS. 75 µL per well of iScript RT-qPCR sample preparation reagent (Bio-Rad #1708898) was added and incubated for 2 minutes before lysate was collected for mRNA analysis. RT-qPCR was set up in triplicate using 3 µL of lysate plus 7 µL of pre-mixed SensiFAST™ SYBR No-ROX One-Step Kit reagents (BIOLINE, Cat# BIO-98005) per reaction. The RT-qPCR reactions were run on a Bio-Rad CFX384 Real-Time PCR System and data was plotted with GraphPad Prism 8 software.

### RNA Extraction and RT-qPCR Analysis

Adherent cells or cell pellets harvested for mRNA analysis were first washed once with PBS and then resuspended in 600 µL of RLT lysis buffer (Qiagen Inc.) supplemented with 1% of betamercaptoethanol. RNA was extracted by QIAcube using the RNeasy Mini QIAcube kit (Qiagen#74116) according to manufacturer’s instructions. The extracted RNA samples were then quantified using a Nanodrop 8000 Spectrophotometer. RT-qPCR reactions were set up in triplicate using 3 µL of 3 ng/ µL of RNA in 7 µL of pre-mixed SensiFAST™ SYBR No-ROX One-Step Kit reagents (BIOLINE# BIO-98005). The RT-qPCR reactions were run on a Bio-Rad CFX384 Real-Time PCR System and data was plotted with GraphPad Prism 8 software.

Primer sequences for RT-qPCR

| Species | Genes | SEQ ID NO. | Forward Primers |
|---|---|---|---|
| Human | RPL27 | 1 | CTGGAATTGACCGCTACCC |
| | AR | 2 | CTTACACGTGGACGACCAGA |
| | GR | 3 | GTGAAATGGGCAAAGGCAATAC |
| | KLK3 | 4 | TGAGAGAGTGGAGAGTGACAT |
| | KLK4 | 5 | GGAACTCTTGCCTCGTTTCT |
| | NKX3.1 | 6 | GAGAGAGCCTTGGCCATATTC |
| | FKBP5 | 7 | GTACACAGCCAGTAGCCTAAAT |
| | GILZ | 8 | GGCAAAGGAGAAGGGTAGTT |
| | PER1 | 9 | ATCCAGTCCAGCCTTACCTA |
| | KLF9 | 10 | CACCTGACCCATACTCGTTTC |
| | SGK1 | 11 | CAGCACAACATCCACCTT CT |
| Human | RPL27 | 12 | TGGGCATTAGGTGATTGTAGTT |
| | AR | 13 | GCTGTACATCCGGGACTTGT |
| | GR | 14 | CCCAGAGCAAATGCCATAAGA |
| | KLK3 | 15 | TCCAGGACAGAGTGGGTTAT |
| | KLK4 | 16 | GTCATAGAGCTTACTGCAGACC |
| | NKX3.1 | 17 | GAGAGAGCCTTGGCCATATTC |
| | FKBP5 | 18 | GAGAGCCATGCTCAATCTGT |
| | GILZ | 19 | ACAGTCGTTGTCAGGTGAAG |
| | PER1 | 20 | TTGGGACATAGGAGAAGAAAGC |
| | KLF9 | 21 | CTGTGCTAGTGATGGCTGTT |
| | SGK1 | 22 | CAGGCCATACAGCATCTCATAC |

### Western Blot Analysis

Cell pellets were washed with PBS and lysed in RIPA buffer (VWR#AAJ63306-AK) supplemented with proteasome inhibitor cocktails (VWR #AAJ65789-LQ). Resuspended cells were briefly sonicated for ~30 seconds using a probe sonicator and incubated on ice for 40 minutes, followed with centrifugation at 12,000 rpm for 15 minutes at 4° C. to collect the supernatant. Protein concentration was quantitated using Quick Start™ Bradford Protein Assay Kit (Bio-Rad #5000201).

50 µg/lane of protein was separated on NuPAGE™ 4-12% Bis-Tris Protein Gels (ThermoFisher Scientific#NP0335BOX). Proteins were transferred to nitrocellulose membranes using iBlot 2 Dry Blotting System (ThermoFisher#IB210021) at 20 V for 10 minutes. Membrane was first blocked using Intercept Blocking Buffer (LI-COR Biosciences#927-60001) for 1 hour at room temperature, then probed with primary antibodies diluted in blocking buffer supplemented with 1% of Tween-20 (BIO-RAD#1610781) and incubated overnight at 4° C. Next, membranes were washed 3 × 5 minutes with 1X TBS (Bio-Rad #1706435) supplemented with 1% Tween 20 (TBST) on shaker, then probed with secondary antibody at room temperature for 1 hour. Last, membrane was washed 3 × 10 minutes with TBST and one time with TBS for 10 minutes, and then developed using Odyssey CLx Imaging System (LI-COR Biosciences#9140). Primary antibodies: AR (CST#5153S), GR (CST#12041), Actin (CST#8457).

### PSA AlphaLISA Assay

Cell supernatant was collected from the experiment groups for various treatment conditions and time. PSA levels were determined using PSA (human) AlphaLISA Detection Kit (PerkinElmer #AL228C) according to manufacturer’s instructions. Specifically, 5 µL of sample was added to 20 µL with 10 µg/mL of AlphaLISA Anti-Analyte Acceptor beads and 1 nM of Biotinylated Antibody Anti-Analyte in a 96-well plate (Greiner#781904) and incubated for 1 hour at room temperature. 25 µL with 40 µg/mL of 2X Streptavidin Donor beads was then added and incubated for 30 minutes in the dark at room temperature. The plate was then read on CLARIOstar^{Plus} (BMG LABTECH). A standard curve was generated using the human PSA analyte provided in the kit by plotting AlphaLISA counts vs concentration of PSA (pg/mL). The standard curve was used to determine unknown PSA concentration from the experiment samples. The measured PSA concentrations were converted into concentration of ng per mL (ng/mL) and data was plotted in GraphPad Prism 8.

### Example 6. PROTAC® AR Degrader ARV-110 Eliminates AR Protein and Inhibits AR Target Gene Expression

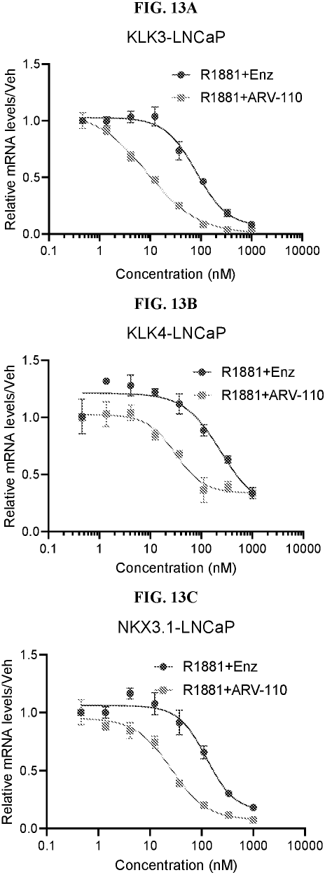

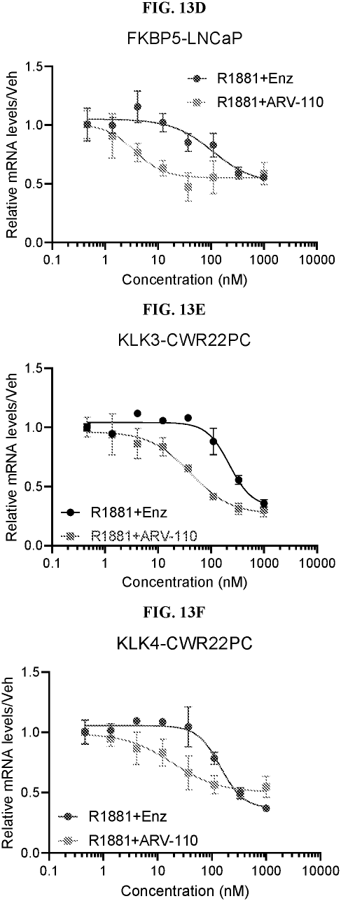

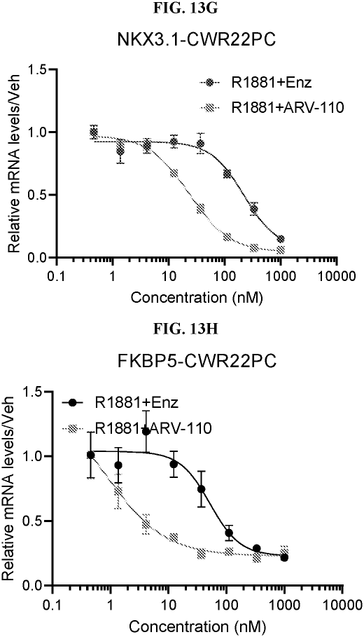

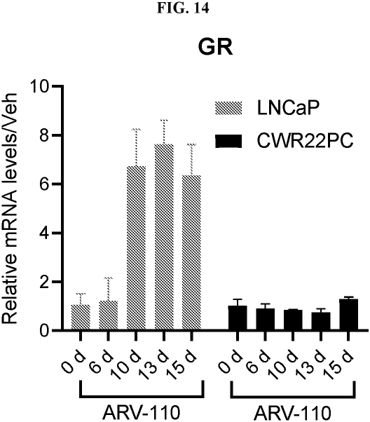

PROTAC® AR degrader ARV-110 (FIG. 12A) was synthesized using methods known to those having ordinary skill in the art. To verify the ability of ARV-110 compound to degrade AR protein, LNCaP and CWR22PC prostate cancer cells were plated in RPMI-1640 media supplemented with 10% charcoal-stripped serum (CSS). After 24 hours, the cells were treated with vehicle (untreated, UT) or with 3, 10, 30, 100, 300 and 1000 nM of ARV-110 and incubated at 37° C. for 3 days. Treated cells were then trypsinized and harvested to measure AR and GR protein by western blot analysis and mRNA levels by RT-qPCR analysis. The results showed that ARV-110 degrades AR protein in a dose-dependent manner (FIG. 12B), while minimally impacting GR protein (FIG. 12B) and AR transcript levels (FIG. 12C). To further solidify ARV-110 as AR degrader, the impact of ARV-110 on AR target gene expression was measured using target gene assays with 8-point dilution for enzalutamide and ARV-110 and concentrations ranging from 1 µM to 0.46 nM. In both LNCaP and CWR22PC cells, ARV-110 inhibits the expression of AR target genes including KLK3, KLK4, NKX3.1, and FKBP5 in a dose-dependent manner with greater potency than AR antagonist enzalutamide (Enz) (FIG. 13A - FIG. 13H). These data indicate that ARV-110 reduces AR protein levels through protein degradation and potently blocks AR signaling in preclinical prostate cancer models.

### Example 7. GR mRNA and GR Signaling are Upregulated in a Time Dependent Manner in CRPC Cells in Response to Treatment With AR Degrader ARV-110

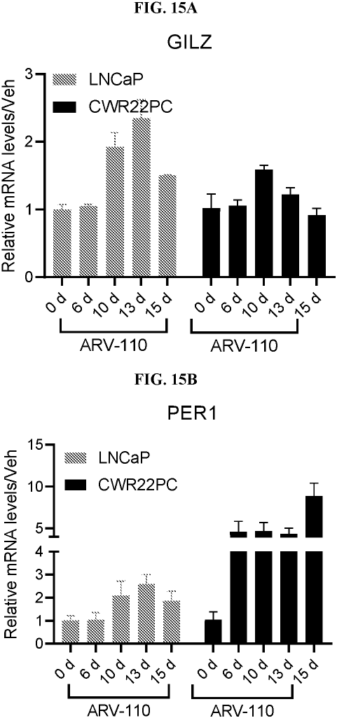

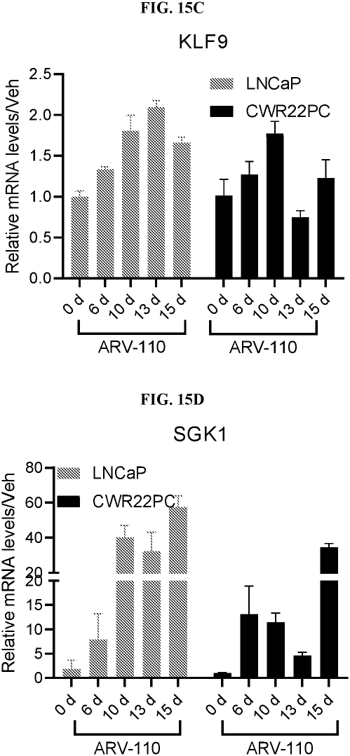

To explore the potential impact of chronic treatment with ARV-110 on GR levels, LNCaP and CWR22PC cells were seeded in CSS media supplemented with 30 nM of GR agonist dexamethasone, treated with 100 nM of ARV-110 for 0, 6, 10, 13, and 15 days, and harvested for RT-qPCR analysis. The results showed that ARV-110 treatment upregulates GR mRNA levels (~7-fold) in GR-negative LNCaP cells (FIG. 14). The effect was minimal in CWR22PC cells that already moderately express GR pre-treatment (FIG. 14). Concurrently, the expression of GR target genes including GILZ, PER1, KLF9, and SGK1 in LNCaP and CWR22PC cells post treatment with ARV-110 were measured. The results showed that most of the GR target genes are upregulated in a time dependent manner in both cell lines post ARV-110 treatment (FIG. 15A - FIG. 15D), suggesting that chronic AR degrader treatment not only upregulates GR but also activates or enhances GR signaling in prostate cancer cells.

### Example 8. Compound 1 Reverses Glucocorticoid-Induced AR and/or GR Target Genes in Response to Treatment With ARV-110

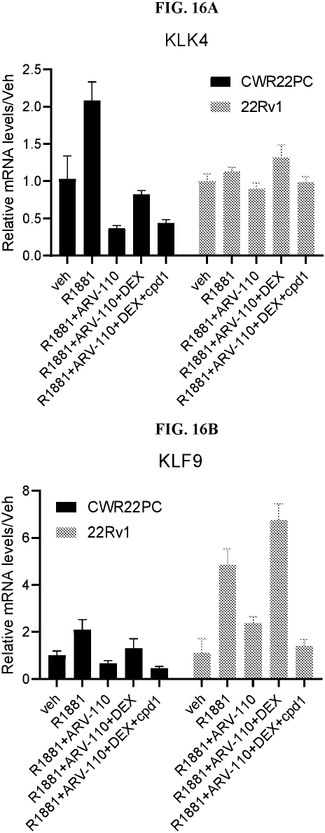

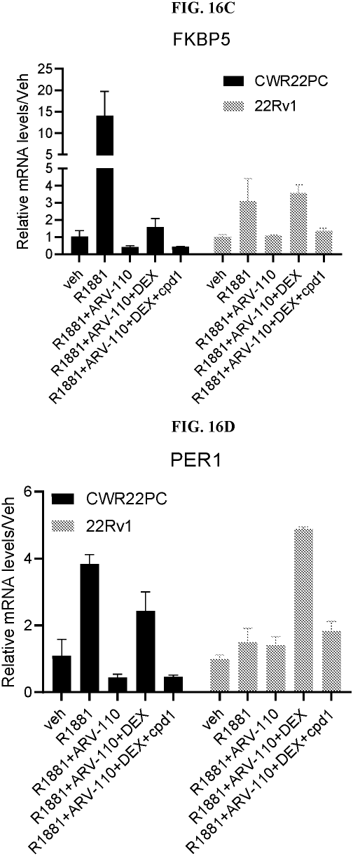

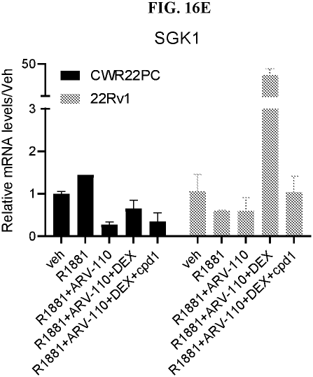

GR-moderate CWR22PC and 22Rv1 prostate cancer cells were seeded at a density of 2 × 10⁵ cells/well in 6-well plates in 2 mL CSS media, and treated with vehicle, 0.1 nM of R1881, 1 µM of ARV-110, 30 nM of dexamethasone (DEX), 0.5 µM of compound 1, or combinations thereof (FIG. 16A - FIG. 16E), to investigate whether GR rescues AR signaling blocked by ARV-110 and whether ARV-110 in combination with GR antagonist compound 1 achieves complete signaling inhibition in response to both androgen and glucocorticoid stimulation. 48 hours post treatment, RNA was extracted, and RT-qPCR analysis was performed to measure the transcript levels of AR and/or GR target genes including KLK4, KLF9, FKBP5, PER1, and SGK1. The results showed that the expression induction by R1881 for AR targets KLK4 and FKBP5 is completely inhibited by ARV-110 treatment. Furthermore, activation of GR with synthetic glucocorticoid dexamethasone (DEX) rescues a portion of AR targets such as KLK4 and FKBP5, and induces GR targets such as KLF9, PER1, and SGK1. Treatment with GR antagonist Compound 1 fully reverses DEX-induced GR and AR target gene expression in the presence of ARV-110. The data suggest that in GR-positive prostate models, beyond activating GR signaling, glucocorticoids also induce a subset of AR target genes upon AR protein loss from AR degrader treatment, and these effects are fully reversed with compound 1 co-treatment.

### Example 9. Compound 1 Overcomes Glucocorticoid-Mediated Resistance to Treatment With ARV-110

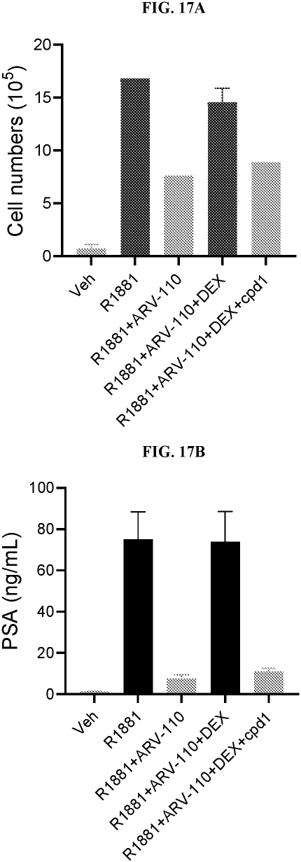

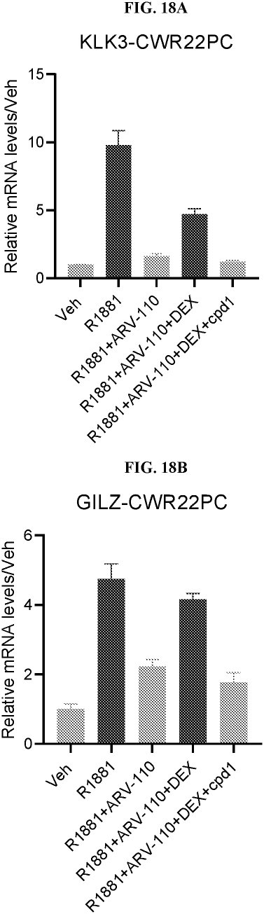

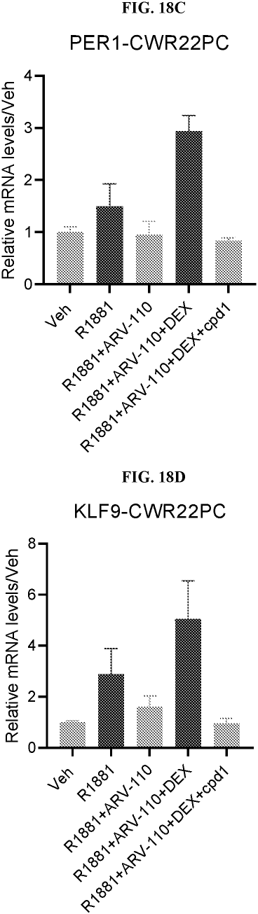

The functional consequences of GR upregulation on prostate cancer cells were investigated. CWR22PC cells were seeded at a density of 2 × 10⁵ cells/well in 6-well plates in 2 mL CSS media. After 24 hours, cells were treated with vehicle, 0.1 nM of R1881, 1 µM of ARV-110, 30 nM of dexamethasone (DEX), 0.5 µM of compound 1, or combinations thereof as indicated in FIG. 17A and FIG. 17B. Fresh media with treatments was replaced every 7 days, and cells were maintained at 37° C. for 21 days before final harvest. Cell supernatants were harvested for secreted PSA measurement using the PSA AlphaLISA assay (FIG. 17B). On day 21 at end of study (EOS), cells were trypsinized and resuspended for cell number counting, while the remaining cells were pelleted and used for RNA extraction and EOS biomarker analysis using RT-qPCR target gene assays. The results showed that ARV-110 treatment blunts R1881-induced cell growth (FIG. 17A), PSA secretion (FIG. 17B), and target gene expression (FIG. 18A - FIG. 18D). 30 nM of dexamethasone almost completely rescues the effect of ARV-110 treatment by restoring cell growth, PSA secretion, and biomarker expression. Importantly, the addition of compound 1 fully reverses these effects. These results indicate that GR may be a resistance mechanism to AR degraders such as ARV-110, and that compound 1 in combination with AR degraders achieves better efficacy when GR signaling is active in preclinical prostate cancer models.

1. A method of treating prostate cancer in a subject in need thereof, the method comprising administering to the subject a therapeutically effective amount of (i) a glucocorticoid receptor (GR) antagonist and (ii) an androgen receptor (AR) degrader.

2. The method of claim 1, wherein one or more cells comprising the prostate cancer in the subject exhibits an elevated expression of glucocorticoid receptor (GR) prior to the administration of the therapeutically effective amount of the glucocorticoid receptor (GR) antagonist and the androgen receptor (AR) degrader.

3. The method of claim 1, wherein the glucocorticoid receptor (GR) antagonist and the androgen receptor (AR) degrader are administered to the subject in need thereof concurrently.

4. The method of claim 1, wherein the glucocorticoid receptor (GR) antagonist and the androgen receptor (AR) degrader are administered to the subject in need thereof consecutively.

5. The method of claim 1, wherein the prostate cancer in the subject is metastatic prostate cancer.

6. The method of claim 1, wherein the prostate cancer in the subject is metastatic castration-resistant prostate cancer.

7. The method of claim 1, wherein the prostate cancer in the subject is localized high risk prostate cancer, recurrent prostate cancer, non-metastatic CRPC (nmCRPC), non-metastatic castration-sensitive prostate cancer, or metastatic castration-sensitive prostate cancer.

8. The method of claim 1, wherein the glucocorticoid receptor (GR) antagonist is Compound 1: 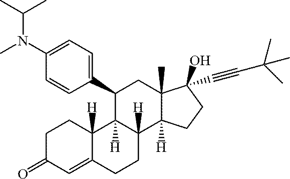
or a pharmaceutically acceptable salt thereof.

9. The method of claim 1, wherein the glucocorticoid receptor (GR) antagonist is mifepristone, cyproterone acetate, relacorilant (CORT125134), exicorilant (CORT125281), miricorilant (CORT118335), CORT113176, CORT108297, PT150 (formerly Org34517), PT157, or PT162.

10. The method of claim 1, wherein the androgen receptor (AR) degrader is ARV-110 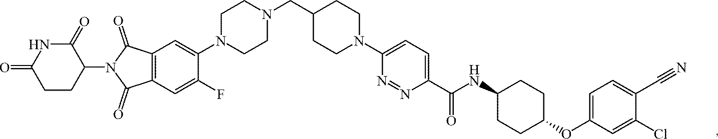
or a pharmaceutically acceptable salt, solvate, or stereoisomer thereof.

11. A glucocorticoid receptor (GR) antagonist for use in combination with an androgen receptor (AR) degrader in a method of treating prostate cancer in a subject.

12. The glucocorticoid receptor (GR) antagonist for use in combination of claim 11, wherein the prostate cancer in the subject is metastatic prostate cancer.

13. The glucocorticoid receptor (GR) antagonist for use in combination of claim 11, wherein the prostate cancer in the subject is metastatic castration-resistant prostate cancer.

14. The glucocorticoid receptor (GR) antagonist for use in combination of claim 11, wherein the prostate cancer in the subject is localized high risk prostate cancer, recurrent prostate cancer, non-metastatic CRPC (nmCRPC), non-metastatic castration-sensitive prostate cancer, or metastatic castration-sensitive prostate cancer.

15. The glucocorticoid receptor (GR) antagonist for use in combination of claim 11, wherein the glucocorticoid receptor (GR) antagonist is Compound 1: 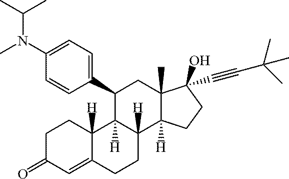
or a pharmaceutically acceptable salt thereof.

16. The glucocorticoid receptor (GR) antagonist for use in combination of claim 11, wherein the glucocorticoid receptor (GR) antagonist is mifepristone, cyproterone acetate, relacorilant (CORT125134), exicorilant (CORT125281), miricorilant (CORT118335), CORT113176, CORT108297, PT150 (formerly Org34517), PT157, or PT162.

17. The glucocorticoid receptor (GR) antagonist for use in combination of claim 11, wherein the androgen receptor (AR) degrader is ARV-110 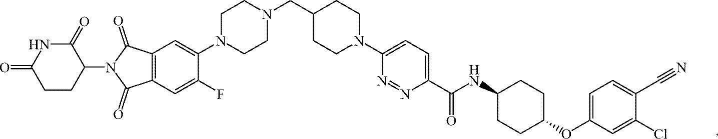
or a pharmaceutically acceptable salt, solvate, or stereoisomer thereof.
